(19) United States
(12) Patent Application Publication
(10) Pub. No.: US 2017/0226059 A1
(43) Pub. Date: Aug. 10, 2017

(54) APOE4-TARGETED THERAPUTICS THAT INCREASE SIRT1

(71) Applicants: Barbara JAGODZINSKA, Redwood City, CA (US); Jesus CAMPAGNA, Los Angeles, CA (US); Johnny PHAM, Los Angeles, CA (US); Michael E. JUNG, Los Angeles, CA (US); Varghese JOHN, Los Angeles, CA (US); Rammohan RAO, Novato, CA (US); Dale E. BREDESEN, Los Angeles, CA (US); The Regents of the University of California, Oakland, CA (US); Buck Institute for Research on Aging, Novato, CA (US)

(72) Inventors: Barbara Jagodzinska, Redwood City, CA (US); Jesus Campagna, Los Angeles, CA (US); Johnny Pham, Los Angeles, CA (US); Michael E. Jung, Los Angeles, CA (US); Varghese John, San Francisco, CA (US); Rammohan Rao, Novato, CA (US); Dale E. Bredesen, Los Angeles, CA (US); Veena P. Theendakara, Petaluma, CA (US)

(21) Appl. No.: 15/500,503

(22) PCT Filed: Aug. 19, 2015
(86) PCT No.: PCT/US2015/045928

§ 371 Date: Jan. 30, 2017

Related U.S. Application Data
(60) Provisional application No. 62/039,024, filed on Aug. 19, 2014.

(51) Int. Cl. C07D 211/60; C07D 207/16; C07C 229/26; C07C 237/06; C07C 229/08; C07C 229/28

(52) U.S. Cl. CPC C07D 211/60; C07C 229/08; C07C 229/28; C07C 229/26; C07C 237/06; C07D 207/16; C07C 2601/04; C07C 2601/14

(57) ABSTRACT

A link between ApoE4 allele and sirtuins expression level is identified that is believed to be associated with elevated risk for the promotion of processing of amyloid precursor protein (APP) by the non-amyloidogenic pathway in a mammal leading to elevated risk for Alzheimer's disease. Compounds are identified that upregulate sirtuins (e.g., SirT1) expression levels and appear to be useful in the treatment and/or prophylaxis of MCI and/or Alzheimer's disease.

### CROSS-REFERENCE TO RELATED APPLICATIONS

This application claims benefit of and priority to U.S. Ser. No. 62/039,024, filed on Aug. 19, 2014, which is incorporated herein by reference in its entirety for all purposes.

### STATEMENT OF GOVERNMENTAL SUPPORT

[Not Applicable]

### BACKGROUND

Alzheimer's disease (AD) is estimated to afflict more than 20 million people worldwide and is believed to be the most common cause of dementia. As the World population ages, the number of people with Alzheimer's disease (AD, currently approximately 5.4 million in the United States, will continue to rise. Alzheimer's is a neurodegenerative disease associated with progressive dementia and memory loss. Key characteristics of AD are the accumulation of extracellular deposits containing aggregated Aβ peptide, the development of neurofibrillary tangles in patient's brains, and neuronal synaptic loss in the AD in specific brain regions. AD patients suffer from deficits in cognition, learning and memory, and exhibit impaired long-term potentiation (LTP)(8) and a consistent deficit in cholinergic neurotransmission. Although AD pathogenesis is complex, compelling genetic and biochemical evidence suggest that overproduction of Aβ, or failure to clear this peptide is the earliest event in the amyloid cascade that lead to AD primarily through amyloid deposition, which is presumed to be involved in neurofibrillary tangle formation, neuronal dysfunction and microglia activation, that characterize AD-affected brain tissues.

The amyloid cascade hypothesis (Hardy and Allsop (1991) Trends Pharmacol. Sci., 12: 383-388; Selkoe (1996) J. Biol. Chem., 271: 18295-18298; Hardy (1997) Trends Neurosci., 20: 154-159; Hardy and Selkoe (2002) Science, 297: 353-356) states that overproduction of Aβ, or failure to clear this peptide, leads to AD, primarily through amyloid deposition, which is presumed to be involved in the formation of neurofibrillary tangles, neuronal dysfunction, and microglia activation, that are hallmarks of AD-affected brain tissues (Busciglio et al. (1995) Neuron, 14: 879-888; Gotz et al. (1995) EMBO J., 14: 1304-1313; Lewis et al. (2001) Science, 293: 1487-1491; Hardy et al. (1985) Nat. Neurosci., 1: 355-358).

Considering the causative role of Aβ in AD etiology, novel therapeutic strategies that lower Aβ levels or prevent the formation of the neurotoxic Aβ species have been suggested as a method to prevent or slow the progression of the disease. Indeed, the major focus over the last decade has been to inhibit brain Aβ production and aggregation, to increase parenchymal Aβ clearance, and to interfere with Aβ-induced cell death.

The dominant genetic risk factor for Alzheimer's disease (AD) is the epsilon-4 (ε4) allele of apolipoprotein E (ApoE4), which is present in about two-thirds of AD patients. However, the link between ApoE4 and the pathogenesis of AD has not been characterized or understood.

Three of the four commonly used FDA-approved treatments for AD-donepezil, galantamine, and rivastigmine—provide a modest reduction in the cognitive decline of AD patients. These compounds act by enhancing the activity of the neurotransmitter acetylcholine. The fourth FDA-approved treatment is memantine, which acts by blocking the NMDA receptor.

These agents are approved for the treatment of patients with mild-to-moderate AD, but are most effective when given in the early stages of the disease. Despite some clinical success of these therapeutic agents, the beneficial effects can only be maintained for up to 36 months (Racchi et al. (2004) Pharmacol. Res. 50(4): 441-451). No disease modifying drugs have been approved for clinical use that specifically target the cellular mechanisms of AD—namely the generation of neurotoxic Aβ, p-tau or ApoE4-related changes that precipitate onset of the disease (Bredesen and John (2013) EMBO Mol. Med. 5(6):795-798).

### SUMMARY

The data provided herein link for the first time the ApoE4 allele, a major risk factor for Alzheimer's disease (AD) with expression levels of sirtuins (e.g., SirT1), major longevity determinants, and identify the first candidate therapeutics that target this new link. In initial screens described herein the drug alaproclate was identified as a drug candidate that can reverse the reduction of SirT1 levels, and is highly brain permeable. A dose response analysis of alaproclate showed that alaproclate can increase SirT1 levels in a dose responsive manner.

Alaproclate was also shown to increase the biomarker sAPPα that is decreased in the presence of ApoE4. Alaproclate also decreased p-Tau levels in SHSY5Y. Alaproclate also increased the mRNA for SirT1 and ADAM10 a protease involved in the production of sAPPα.

Various analogs of alaproclate (alaproclate “related” compounds) believed to have similar or greater efficacy were also identified.

It is believed that alaproclate and the other active agents described herein are able inhibit the neurotoxic effects of ApoE4 by increasing the levels of the mediator SirT1, and reverse the decrease in the anti-AD biomarkers such as sAPPα and improve memory. It is believed the active agents described herein can delay the onset of MCI, and/or the progression of MCI to AD, and/or the onset of AD in subjects with the ApoE4 allele. It is also believed the active agents described herein represent the first drugs for the prophylactic and/or therapeutic treatment of subjects with the ApoE4 risk factor.

Various embodiments contemplated herein may include, but need not be limited to, one or more of the following:

### Embodiment 1

A compound according to the formula:

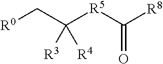

or a pharmaceutically acceptable salt thereof, wherein: R⁸ is selected from the group consisting of

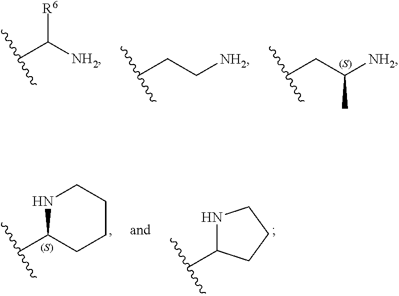

R⁰ is a substituted or unsubstituted cyclic or heterocycle selected from the group consisting of pyridine, pyrimidine, naphthalene, quinolone, isoquinoline, cinnoline, phenyl, substituted phenyl, oxazole, furan, isoxazole, thiazole, thiophene, pyrole, pyrazole, and imidazole; R³ and R⁴ are independently selected from the group consisting of hydrogen, methyl, ethyl, propyl, and butyl, or R³ taken with R⁴ and the carbon joining R³ and R⁴ form cyclohexane or cyclobutane; R⁵ is selected from the group consisting of O, NH, and NHR⁷, where R⁷ is a C1-C5 alkyl, or a cycloalkyl; R⁶ is selected from the group consisting the R-group (side chain) of one of the 20 natural amino acids, phenylglycine, and norleucine; and R⁶ is not CH₃, or R³ and R⁴ are not both CH₃, or when R⁶ is CH₃, said compound is not a compound selected from the group consisting of

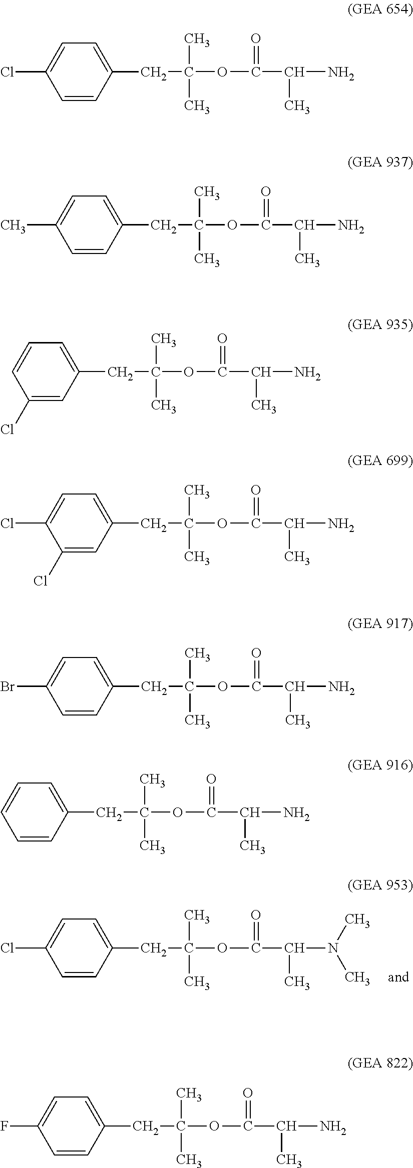

### Embodiment 2

The compound of embodiment 1, wherein said compound is not any of compounds 1, 2, 4, 5, 6, 7, 8, 11, and 15 in Table 6.

### Embodiment 3

The according to any one of embodiments 1-2, wherein said compound is a compound according to the formula

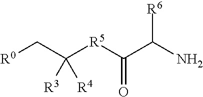

or a pharmaceutically acceptable salt thereof.

### Embodiment 4

The compound according to any one of embodiment 1-3, wherein R³ and R⁴ are independently selected from the group consisting of hydrogen, methyl, ethyl, propyl, and butyl.

### Embodiment 5

The according to any one of embodiments 1-4, wherein said compound has the formula

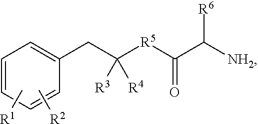

or a pharmaceutically acceptable salt thereof, wherein R¹ and R² are independently selected from the group consisting of hydrogen, halogen, alkyl having 1, 2 or 3 carbon atoms, and alkoxy having 1, 2 or 3 carbon atoms.

### Embodiment 6

The compound of embodiment 5, wherein said compound has the Formula

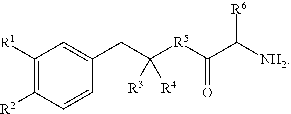

### Embodiment 7

The compound according to any one of embodiments 1-6, wherein R³ is CH₃.

### Embodiment 8

The compound of embodiment 7, wherein R⁴ is H.

### Embodiment 9

The compound of embodiment 7, wherein R⁴ is CH₃.

### Embodiment 10

The compound according to any one of embodiments 1-9, wherein R⁵ is O.

### Embodiment 11

The compound according to any one of embodiments 1-9, wherein R⁵ is NHR⁷.

### Embodiment 12

The compound according to any one of embodiments 1-9, wherein R⁵ is NH.

### Embodiment 13

The compound according to any one of embodiments 1-12, wherein R¹ and R² are independently selected from the group consisting of hydrogen, halogen, and CH₃.

### Embodiment 14

The compound of embodiment 13, wherein R¹ and R² are independently selected from the group consisting of H, Cl, and F.

### Embodiment 15

The compound of embodiment 13, wherein R¹ and R² are both Cl.

### Embodiment 16

The compound of embodiment 13, wherein R¹ and R² are both F.

### Embodiment 17

The compound of embodiment 13, wherein R¹ is Cl and R² is F, or R¹ is F and R² is Cl.

### Embodiment 18

The compound of embodiment 13, wherein R¹ is H and R² is F.

### Embodiment 19

The compound of embodiment 13, wherein R¹ is H and R² is Cl.

### Embodiment 20

The compound of embodiment 13, wherein R¹ is H and R² is CH₃.

### Embodiment 21

The compound according to any one of embodiments 1-20, wherein R⁶ is selected from the group consisting of H, CH₃, —CH(CH₃)₂, —CH₂—CH(CH₃)₂, —CH₂-phenyl, CH2-substituted phenyl, —CH(CH₃)—CH₂CH₃, -phenyl, substituted phenyl, and —CH₂—CH₂—CH₂—CH₃.

### Embodiment 22

The compound according to any one of embodiments 1-20, wherein R⁶ is H.

### Embodiment 23

The compound according to any one of embodiments 1-20, wherein R⁶ is CH₃.

### Embodiment 24

The compound according to any one of embodiments 1-20, wherein R⁶ is —CH(CH₃)₂.

### Embodiment 25

The compound according to any one of embodiments 1-20, wherein R⁶ is —CH₂—CH(CH₃)₂.

### Embodiment 26

The compound according to any one of embodiments 1-20, wherein R⁶ is —CH₂-phenyl.

### Embodiment 27

The compound according to any one of embodiments 1-20, wherein R⁶ is —CH(CH₃)—CH₂CH₃.

### Embodiment 28

The compound according to any one of embodiments 1-20, wherein R⁶ is -phenyl.

### Embodiment 29

The compound according to any one of embodiments 1-20, wherein R⁶ is —CH₂—CH₂—CH₂—CH₃.

### Embodiment 30

The compound of embodiment 1, wherein said compound includes any one of compounds 3, 9, 10, 12, 13, 14, 16, 17, 18, 19, 20, 21, 22, 23, or 24 in Table 6, or a pharmaceutically acceptable salt thereof.

### Embodiment 31

The compound of embodiment 30, wherein said compound is compound 3 in Table 6, or a pharmaceutically acceptable salt thereof.

### Embodiment 32

The compound of embodiment 30, wherein said compound is compound 9 in Table 6, or a pharmaceutically acceptable salt thereof.

### Embodiment 33

The compound of embodiment 30, wherein said compound is compound 10 in Table 6, or a pharmaceutically acceptable salt thereof.

### Embodiment 34

The compound of embodiment 30, wherein said compound is compound 12 in Table 6, or a pharmaceutically acceptable salt thereof.

### Embodiment 35

The compound of embodiment 30, wherein said compound is compound 13 in Table 6, or a pharmaceutically acceptable salt thereof.

### Embodiment 36

The compound of embodiment 30, wherein said compound is compound 14 in Table 6, or a pharmaceutically acceptable salt thereof.

### Embodiment 37

The compound of embodiment 30, wherein said compound is compound 16 in Table 6, or a pharmaceutically acceptable salt thereof.

### Embodiment 38

The compound of embodiment 30, wherein said compound is compound 17 in Table 6, or a pharmaceutically acceptable salt thereof.

### Embodiment 39

The compound of embodiment 30, wherein said compound is compound 18 in Table 6, or a pharmaceutically acceptable salt thereof.

### Embodiment 40

The compound of embodiment 30, wherein said compound is compound 19 in Table 6, or a pharmaceutically acceptable salt thereof.

### Embodiment 41

The compound of embodiment 30, wherein said compound is compound 20 in Table 6, or a pharmaceutically acceptable salt thereof.

### Embodiment 42

The compound of embodiment 30, wherein said compound is compound 21 in Table 6, or a pharmaceutically acceptable salt thereof.

### Embodiment 43

The compound of embodiment 30, wherein said compound is compound 22 in Table 6, or a pharmaceutically acceptable salt thereof.

### Embodiment 44

The compound of embodiment 30, wherein said compound is compound 23 in Table 6, or a pharmaceutically acceptable salt thereof.

### Embodiment 45

The compound of embodiment 30, wherein said compound is compound 24 in Table 6, or a pharmaceutically acceptable salt thereof.

### Embodiment 46

The compound according to any one of embodiments 1-45, wherein said compound is a substantially pure S enantiomer.

### Embodiment 47

The compound according to any one of embodiments 1-45, wherein said compound is a substantially pure R enantiomer.

### Embodiment 48

A pharmaceutical formulation including one or more compounds according to any one of embodiments 1-47 and a pharmaceutically acceptable diluent or excipient.

### Embodiment 49

The pharmaceutical formulation of embodiment 48, wherein said compound is compound 1 in Table 6, or a pharmaceutically acceptable salt thereof.

### Embodiment 50

The pharmaceutical formulation of embodiment 48, wherein said compound is compound 2 in Table 6, or a pharmaceutically acceptable salt thereof.

### Embodiment 51

The pharmaceutical formulation of embodiment 48, wherein said compound is compound 3 in Table 6, or a pharmaceutically acceptable salt thereof.

### Embodiment 52

The pharmaceutical formulation of embodiment 48, wherein said compound is compound 4 in Table 6, or a pharmaceutically acceptable salt thereof.

### Embodiment 53

The pharmaceutical formulation of embodiment 48, wherein said compound is compound 5 in Table 6, or a pharmaceutically acceptable salt thereof.

### Embodiment 54

The pharmaceutical formulation of embodiment 48, wherein said compound is compound 6 in Table 6, or a pharmaceutically acceptable salt thereof.

### Embodiment 55

The pharmaceutical formulation of embodiment 48, wherein said compound is compound 7 in Table 6, or a pharmaceutically acceptable salt thereof.

### Embodiment 56

The pharmaceutical formulation of embodiment 48, wherein said compound is compound 8 in Table 6, or a pharmaceutically acceptable salt thereof.

### Embodiment 57

The pharmaceutical formulation of embodiment 48, wherein said compound is compound 9 in Table 6, or a pharmaceutically acceptable salt thereof.

### Embodiment 58

The pharmaceutical formulation of embodiment 48, wherein said compound is compound 10 in Table 6, or a pharmaceutically acceptable salt thereof.

### Embodiment 59

The pharmaceutical formulation of embodiment 48, wherein said compound is compound 11 in Table 6, or a pharmaceutically acceptable salt thereof.

### Embodiment 60

The pharmaceutical formulation of embodiment 48, wherein said compound is compound 12 in Table 6, or a pharmaceutically acceptable salt thereof.

### Embodiment 61

The pharmaceutical formulation of embodiment 48, wherein said compound is compound 13 in Table 6, or a pharmaceutically acceptable salt thereof.

### Embodiment 62

The pharmaceutical formulation of embodiment 48, wherein said compound is compound 14 in Table 6, or a pharmaceutically acceptable salt thereof.

### Embodiment 63

The pharmaceutical formulation of embodiment 48, wherein said compound is compound 15 in Table 6, or a pharmaceutically acceptable salt thereof.

### Embodiment 64

The pharmaceutical formulation of embodiment 48, wherein said compound is compound 16 in Table 6, or a pharmaceutically acceptable salt thereof.

### Embodiment 65

The pharmaceutical formulation of embodiment 48, wherein said compound is compound 17 in Table 6, or a pharmaceutically acceptable salt thereof.

### Embodiment 66

The pharmaceutical formulation of embodiment 48, wherein said compound is compound 18 in Table 6, or a pharmaceutically acceptable salt thereof.

### Embodiment 67

The pharmaceutical formulation of embodiment 48, wherein said compound is compound 19 in Table 6, or a pharmaceutically acceptable salt thereof.

### Embodiment 68

The pharmaceutical formulation of embodiment 48, wherein said compound is compound 20 in Table 6, or a pharmaceutically acceptable salt thereof.

### Embodiment 69

The pharmaceutical formulation of embodiment 48, wherein said compound is compound 21 in Table 6, or a pharmaceutically acceptable salt thereof.

### Embodiment 70

The pharmaceutical formulation of embodiment 48, wherein said compound is compound 22 in Table 6, or a pharmaceutically acceptable salt thereof.

### Embodiment 71

The pharmaceutical formulation of embodiment 48, wherein said compound is compound 23 in Table 6, or a pharmaceutically acceptable salt thereof.

### Embodiment 72

The pharmaceutical formulation of embodiment 48, wherein said compound is compound 24 in Table 6, or a pharmaceutically acceptable salt thereof.

### Embodiment 73

The pharmaceutical formulation according to any one of embodiment 48-72, wherein said formulation is a unit dosage formulation.

### Embodiment 74

The formulation according to any one of embodiments 48-73, wherein said formulation is compounded for administration via a route selected from the group consisting of oral delivery, isophoretic delivery, transdermal delivery, parenteral delivery, aerosol administration, administration via inhalation, intravenous administration, and rectal administration.

### Embodiment 75

A method of increasing the expression of SirT1, and/or increasing the expression of ADAM10, and/or increasing sAPPα, and/or decreasing p-tau in a mammal, said method comprising: administering to said mammal an effective amount of one or more compounds according to any one of embodiments 1-47 and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 857),

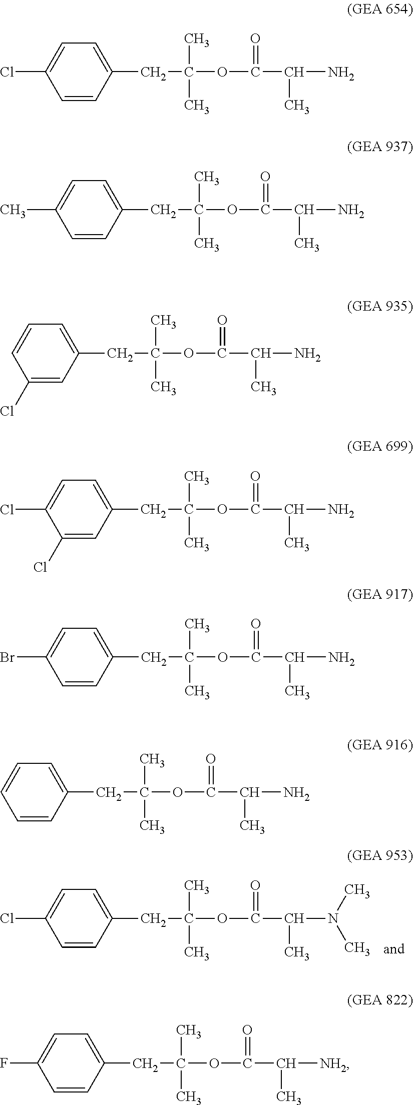

and/or a formulation according to any one of embodiments 48-74.

### Embodiment 76

The method of embodiment 75, wherein said method increases the expression of SirT1 in said mammal.

### Embodiment 77

The method according to any one of embodiments 75-76, wherein said method increases the expression of ADAM10 in said mammal.

### Embodiment 78

The method according to any one of embodiments 75-77, wherein said method increases sAPPα in said mammal.

### Embodiment 79

The method according to any one of embodiments 75-78, wherein said method decreases p-tau in said mammal.

### Embodiment 80

A method of normalizing ApoE4 mediated effects on SirT1, normalizing SirT1/SirT2 ratios, and/or normalizing APP processing in a mammal, said method comprising: administering to said mammal an effective amount of one or more compounds according to any one of embodiments 1-47 and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 857),

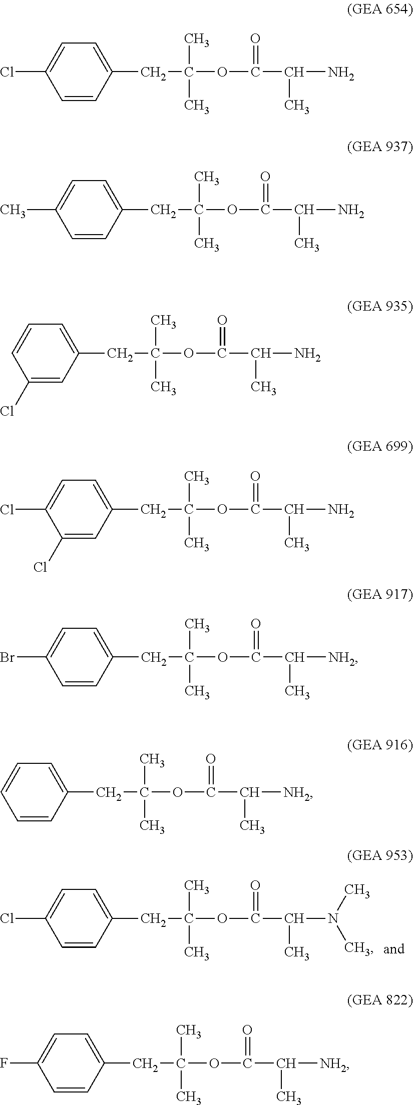

and/or a formulation according to any one of embodiments 48-74.

### Embodiment 81

The method according of embodiment 80, wherein said method increases or normalizes the expression of SirT1 in said mammal.

### Embodiment 82

The method according to any one of embodiments 80-81, wherein said method increases the expression of ADAM10 in said mammal.

### Embodiment 83

A method of promoting the processing of amyloid precursor protein (APP) by the non-amyloidogenic pathway in a mammal, said method comprising: administering to said mammal an effective amount of one or more compounds according to any one of embodiments 1-47 and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 8571,

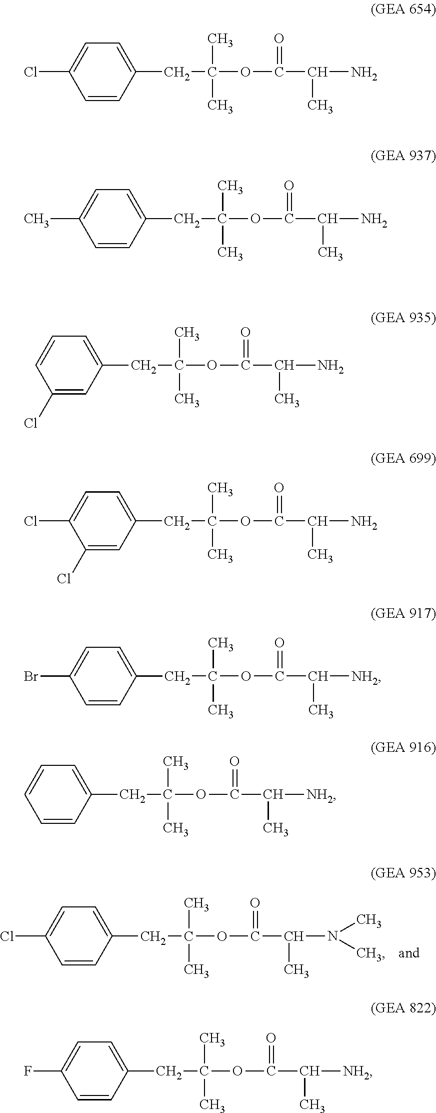

and/or a formulation according to any one of embodiments 48-74.

### Embodiment 84

The method of embodiments 83, wherein said method increases sAPPα in said mammal.

### Embodiment 85

The method according to any one of embodiments 83-84, wherein said method decreases p-tau in said mammal.

### Embodiment 86

A method of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal, said method comprising: administering to said mammal an effective amount of one or more compounds according to any one of embodiments 1-47 and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 857),

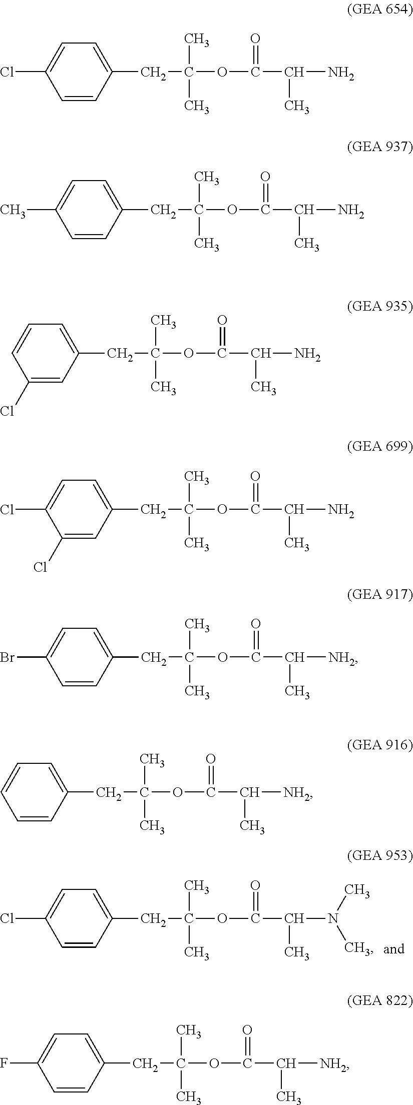

and/or a formulation according to any one of embodiments 48-74.

### Embodiment 87

A method of ameliorating one or more symptoms of Alzheimer's disease, and/or reversing Alzheimer's disease, and/or reducing the rate of progression of Alzheimer's disease in a mammal, said method comprising: administering to said mammal an effective amount of one or more compounds according to any one of embodiments 1-47, and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 857),

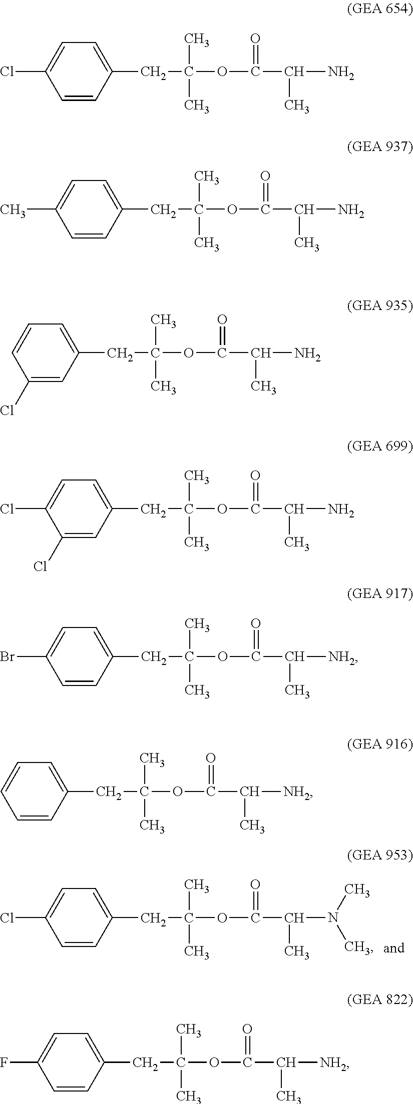

and/or a formulation according to any one of embodiments 48-74.

### Embodiment 88

The method according to any one of embodiments 75-87, wherein said compound includes alaproclate.

### Embodiment 89

The method according to any one of embodiments 75-87, wherein said compound is compound 1 in Table 6, or a pharmaceutically acceptable salt thereof.

### Embodiment 90

The method according to any one of embodiments 75-87, wherein said compound is compound 2 in Table 6, or a pharmaceutically acceptable salt thereof.

### Embodiment 91

The method according to any one of embodiments 75-87, wherein said compound is compound 3 in Table 6, or a pharmaceutically acceptable salt thereof.

### Embodiment 92

The method according to any one of embodiments 75-87, wherein said compound is compound 4 in Table 6, or a pharmaceutically acceptable salt thereof.

### Embodiment 93

The method according to any one of embodiments 75-87, wherein said compound is compound 5 in Table 6, or a pharmaceutically acceptable salt thereof.

### Embodiment 94

The method according to any one of embodiments 75-87, wherein said compound is compound 6 in Table 6, or a pharmaceutically acceptable salt thereof.

### Embodiment 95

The method according to any one of embodiments 75-87, wherein said compound is compound 7 in Table 6, or a pharmaceutically acceptable salt thereof.

### Embodiment 96

The method according to any one of embodiments 75-87, wherein said compound is compound 8 in Table 6, or a pharmaceutically acceptable salt thereof.

### Embodiment 97

The method according to any one of embodiments 75-87, wherein said compound is compound 9 in Table 6, or a pharmaceutically acceptable salt thereof.

### Embodiment 98

The method according to any one of embodiments 75-87, wherein said compound is compound 10 in Table 6, or a pharmaceutically acceptable salt thereof.

### Embodiment 99

The method according to any one of embodiments 75-87, wherein said compound is compound 11 in Table 6, or a pharmaceutically acceptable salt thereof.

### Embodiment 100

The method according to any one of embodiments 75-87, wherein said compound is compound 12 in Table 6, or a pharmaceutically acceptable salt thereof.

### Embodiment 101

The method according to any one of embodiments 75-87, wherein said compound is compound 13 in Table 6, or a pharmaceutically acceptable salt thereof.

### Embodiment 102

The method according to any one of embodiments 75-87, wherein said compound is compound 14 in Table 6, or a pharmaceutically acceptable salt thereof.

### Embodiment 103

The method according to any one of embodiments 75-87, wherein said compound is compound 15 in Table 6, or a pharmaceutically acceptable salt thereof.

### Embodiment 104

The method according to any one of embodiments 75-87, wherein said compound is compound 16 in Table 6, or a pharmaceutically acceptable salt thereof.

### Embodiment 105

The method according to any one of embodiments 75-87, wherein said compound is compound 17 in Table 6, or a pharmaceutically acceptable salt thereof.

### Embodiment 106

The method according to any one of embodiments 75-87, wherein said compound is compound 18 in Table 6, or a pharmaceutically acceptable salt thereof.

### Embodiment 107

The method according to any one of embodiments 75-87, wherein said compound is compound 19 in Table 6, or a pharmaceutically acceptable salt thereof.

### Embodiment 108

The method according to any one of embodiments 75-87, wherein said compound is compound 20 in Table 6, or a pharmaceutically acceptable salt thereof.

### Embodiment 109

The method according to any one of embodiments 75-87, wherein said compound is compound 21 in Table 6, or a pharmaceutically acceptable salt thereof.

### Embodiment 110

The method according to any one of embodiments 75-87, wherein said compound is compound 22 in Table 6, or a pharmaceutically acceptable salt thereof.

### Embodiment 111

The method according to any one of embodiments 75-87, wherein said compound is compound 23 in Table 6, or a pharmaceutically acceptable salt thereof.

### Embodiment 112

The method according to any one of embodiments 75-87, wherein said compound is compound 24 in Table 6, or a pharmaceutically acceptable salt thereof.

### Embodiment 113

The method according to any one of embodiments 75-112, wherein the mammal has a familial risk for having Alzheimer's disease.

### Embodiment 114

The method according to any one of embodiments 75-112, wherein the mammal has a familial Alzheimer's disease (FAD) mutation.

### Embodiment 115

The method according to any one of embodiments 75-112, wherein said mammal has one copy of the ApoE4 allele.

### Embodiment 116

The method according to any one of embodiments 75-112, wherein said mammal has two copies of the ApoE4 allele.

### Embodiment 117

The method according to any one of embodiments 75-116, wherein said mammal is a human.

### Embodiment 118

The method according to any one of embodiments 75-117, wherein, wherein said method is a method of preventing or delaying the transition from a cognitively asymptomatic pre-Alzheimer's condition to a pre-Alzheimer's cognitive dysfunction.

### Embodiment 119

The method according to any one of embodiments 75-117, wherein said method is a method of preventing or delaying the onset of a pre-Alzheimer's cognitive dysfunction.

### Embodiment 120

The method according to any one of embodiments 75-119, wherein said method includes ameliorating one or more symptoms of a pre-Alzheimer's cognitive dysfunction.

### Embodiment 121

The method according to any one of embodiments 75-119, wherein said method includes preventing or delaying the progression of a pre-Alzheimer's cognitive dysfunction to Alzheimer's disease.

### Embodiment 122

The method of embodiment 121, wherein said method delays or prevents the progression of MCI to Alzheimer's disease.

### Embodiment 123

The method according to any one of embodiments 75-121, wherein said mammal exhibits biomarker positivity of Aβ in a clinically normal human mammal age 50 or older.

### Embodiment 124

The method according to any one of embodiments 75-121, wherein said mammal exhibits biomarker positivity of Aβ using PET, and/or CSF analysis, and/or structural MRI (sMRI).

### Embodiment 125

The method according to any one of embodiments 75-121, wherein said mammal exhibits asymptomatic cerebral amyloidosis.

### Embodiment 126

The method according to any one of embodiments 75-121, wherein said mammal exhibits cerebral amyloidosis in combination with downstream neurodegeneration.

### Embodiment 127

The method according to any one of embodiments 75-121, wherein said mammal is cognitively asymptomatic.

### Embodiment 128

The method according to any one of embodiments 75-121, wherein said mammal exhibits cerebral amyloidosis in combination with downstream neurodegeneration and subtle cognitive/behavioral decline.

### Embodiment 129

The method of embodiment 128, wherein said downstream neurodegeneration is determined by one or more elevated markers of neuronal injury selected from the group consisting of tau, and FDG uptake.

### Embodiment 130

The method according to any one of embodiments 125-129, wherein said cerebral amyloidosis is determined by PET, or CSF analysis, and structural MRI (sMRI).

### Embodiment 131

The method according to any one of embodiments 75-121, wherein said mammal is a mammal diagnosed with mild cognitive impairment.

### Embodiment 132

The method of embodiment 131, wherein said mammal shows a clinical dementia rating above zero and below about 1.5.

### Embodiment 133

The method according to any one of embodiments 75-122, wherein the mammal is not diagnosed as at risk for a neurological disease or disorder other than Alzheimer's disease.

### Embodiment 134

The method according to any one of embodiments 75-133, wherein said administration produces a reduction in the CSF of levels of one or more components selected from the group consisting of total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ42/Aβ40 ratio, Aβ42/Aβ38 ratio, sAPPα, sAPPα/sAPPβ ratio, sAPPα/Aβ40 ratio, and sAPPα/Aβ42 ratio.

### Embodiment 135

The method according to any one of embodiments 75-133, wherein said administration produces an increase in plasma levels of SirT1 or normalizes the SirT1/SirT2 ratios.

### Embodiment 136

The method according to any one of embodiments 75-135, wherein said administration produces a reduction of the plaque load in the brain of the mammal.

### Embodiment 137

The method according to any one of embodiments 75-135, wherein said administration produces a reduction in the rate of plaque formation in the brain of the mammal.

### Embodiment 138

The method according to any one of embodiments 75-137, wherein said administration produces an improvement in the cognitive abilities of the mammal.

### Embodiment 139

The method according to any one of embodiments 75-137, wherein said administration produces an improvement in, a stabilization of, or a reduction in the rate of decline of the clinical dementia rating (CDR) of the mammal.

### Embodiment 140

The method according to any one of embodiments 75-139, wherein the mammal is a human and said administration produces a perceived improvement in quality of life by the human.

### Embodiment 141

The method according to any one of embodiments 75-140, wherein the compound(s) are administered via a route selected from the group consisting of oral delivery, isophoretic delivery, transdermal delivery, parenteral delivery, aerosol administration, administration via inhalation, intravenous administration, and rectal administration.

### Embodiment 142

The method according to any one of embodiments 75-140, wherein the compound is administered orally.

### Embodiment 143

The method according to any one of embodiments 75-142, wherein the administering is over a period of at least three weeks.

### Embodiment 144

The method according to any one of embodiments 75-142, wherein the administering is over a period of at least 6 months.

### Embodiment 145

The method according to any one of embodiments 75-144, wherein the compound(s) are formulated for administration via a route selected from the group consisting of isophoretic delivery, transdermal delivery, aerosol administration, administration via inhalation, oral administration, intravenous administration, and rectal administration.

### Embodiment 146

A method of treating diabetes and/or metabolic syndrome, said method including: administering to said mammal an effective amount of one or more compounds according to any one of embodiments 1-47 and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 857),

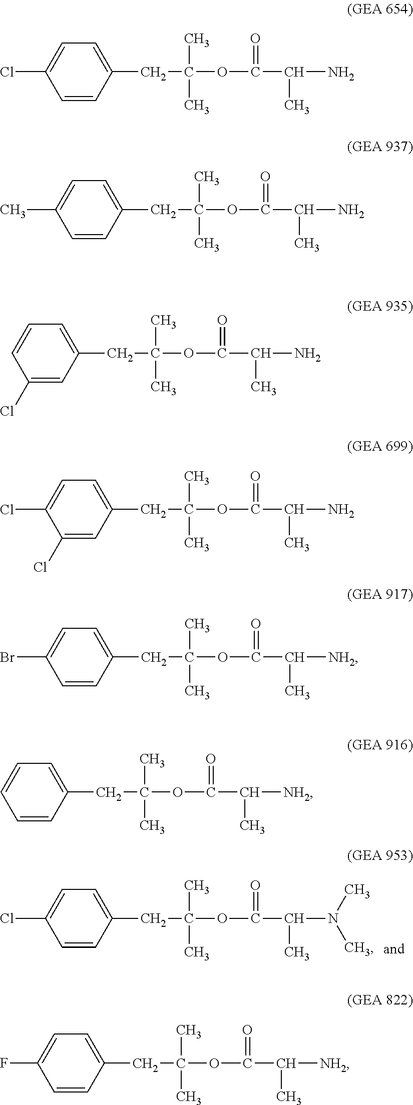

and/or a formulation according to any one of embodiments 48-74.

### Embodiment 147

The method of embodiment 146, wherein said administration produces an increase in plasma levels of SirT1 or normalizes the SirT1/SirT2 ratios.

### Embodiment 148

The method according to any one of embodiments 146-147, wherein said mammal is a human.

### Embodiment 149

The method according to any one of embodiments 146-126, wherein said mammal is clinically obese.

### Embodiment 150

A method of increasing the lifespan and/or healthspan of a mammal, said method including: administering to said mammal an effective amount of one or more compounds according to any one of embodiments 1-47 and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 857),

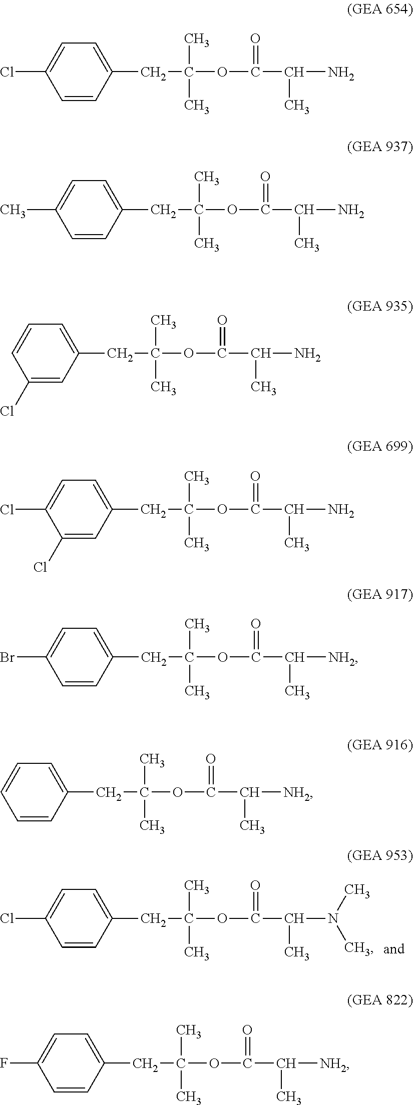

and/or a formulation according to any one of embodiments 48-74.

### Embodiment 151

The method of embodiment 150, wherein said administration produces an increase in plasma levels of SirT1 or normalizes the SirT1/SirT2 ratios.

### Embodiment 152

The method according to any one of embodiments 146-151, wherein said compound includes alaproclate.

### Embodiment 153

The method according to any one of embodiments 146-151, wherein said compound is compound 1 in Table 6, or a pharmaceutically acceptable salt thereof.

### Embodiment 154

The method according to any one of embodiments 146-151, wherein said compound is compound 2 in Table 6, or a pharmaceutically acceptable salt thereof.

### Embodiment 155

The method according to any one of embodiments 146-151, wherein said compound is compound 3 in Table 6, or a pharmaceutically acceptable salt thereof.

### Embodiment 156

The method according to any one of embodiments 146-151, wherein said compound is compound 4 in Table 6, or a pharmaceutically acceptable salt thereof.

### Embodiment 157

The method according to any one of embodiments 146-151, wherein said compound is compound 5 in Table 6, or a pharmaceutically acceptable salt thereof.

### Embodiment 158

The method according to any one of embodiments 146-151, wherein said compound is compound 6 in Table 6, or a pharmaceutically acceptable salt thereof.

### Embodiment 159

The method according to any one of embodiments 146-151, wherein said compound is compound 7 in Table 6, or a pharmaceutically acceptable salt thereof.

### Embodiment 160

The method according to any one of embodiments 146-151, wherein said compound is compound 8 in Table 6, or a pharmaceutically acceptable salt thereof.

### Embodiment 161

The method according to any one of embodiments 146-151, wherein said compound is compound 9 in Table 6, or a pharmaceutically acceptable salt thereof.

### Embodiment 162

The method according to any one of embodiments 146-151, wherein said compound is compound 10 in Table 6, or a pharmaceutically acceptable salt thereof.

### Embodiment 163

The method according to any one of embodiments 146-151, wherein said compound is compound 11 in Table 6, or a pharmaceutically acceptable salt thereof.

### Embodiment 164

The method according to any one of embodiments 146-151, wherein said compound is compound 12 in Table 6, or a pharmaceutically acceptable salt thereof.

### Embodiment 165

The method according to any one of embodiments 146-151, wherein said compound is compound 13 in Table 6, or a pharmaceutically acceptable salt thereof.

### Embodiment 166

The method according to any one of embodiments 146-151, wherein said compound is compound 14 in Table 6, or a pharmaceutically acceptable salt thereof.

### Embodiment 167

The method according to any one of embodiments 146-151, wherein said compound is compound 15 in Table 6, or a pharmaceutically acceptable salt thereof.

### Embodiment 168

The method according to any one of embodiments 146-151, wherein said compound is compound 16 in Table 6, or a pharmaceutically acceptable salt thereof.

### Embodiment 169

The method according to any one of embodiments 146-151, wherein said compound is compound 17 in Table 6, or a pharmaceutically acceptable salt thereof.

### Embodiment 170

The method according to any one of embodiments 146-151, wherein said compound is compound 18 in Table 6, or a pharmaceutically acceptable salt thereof.

### Embodiment 171

The method according to any one of embodiments 146-151, wherein said compound is compound 19 in Table 6, or a pharmaceutically acceptable salt thereof.

### Embodiment 172

The method according to any one of embodiments 146-151, wherein said compound is compound 20 in Table 6, or a pharmaceutically acceptable salt thereof.

### Embodiment 173

The method according to any one of embodiments 146-151, wherein said compound is compound 21 in Table 6, or a pharmaceutically acceptable salt thereof.

### Embodiment 174

The method according to any one of embodiments 146-151, wherein said compound is compound 22 in Table 6, or a pharmaceutically acceptable salt thereof.

### Embodiment 175

The method according to any one of embodiments 146-151, wherein said compound is compound 23 in Table 6, or a pharmaceutically acceptable salt thereof.

### Embodiment 176

The method according to any one of embodiments 146-151, wherein said compound is compound 24 in Table 6, or a pharmaceutically acceptable salt thereof.

In certain embodiments, the compounds, compositions, and pharmaceutical formulations described herein expressly exclude GEA 654, GEA 937, GEA 935, GEA 699, GEA 917, GEA 916, GEA 953, and GEA 822 as described in U.S. Pat. No. 4,237,311 and as identified herein, alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), and 2-(4-chlorophenyl)-1,1-dimethylethyl 2-amino-3-methylbutanoate (GEA 857). In certain embodiments the compounds, compositions, and pharmaceutical formulations alternatively or additionally exclude any one or more of compounds 1 through 24 in Table 6, or compounds 1, 2, 4, 5, 6, 7, 8, 11, and 15 in Table 6, or all of the compounds in Table 6.

### Definitions

Apolipoprotein E (ApoE) is a class of apolipoprotein found in the chylomicron and Intermediate-density lipoprotein (IDLs) that is essential for the normal catabolism of triglyceride-rich lipoprotein constituents. In peripheral tissues, ApoE is primarily produced by the liver and macrophages, and mediates cholesterol metabolism in an isoform-dependent manner. In the central nervous system, ApoE is mainly produced by astrocytes, and transports cholesterol to neurons via ApoE receptors, which are members of the low density lipoprotein receptor gene family. ApoE is polymorphic, with three major isoforms: ApoE2 (cys112, cys158), ApoE3 (cys112, arg158), and ApoE4 (arg112, arg158). The E4 variant is the largest known genetic risk factor for late-onset sporadic Alzheimer disease (AD) in a variety of ethnic groups. Caucasian and Japanese carriers of 2 E4 alleles have between 10 and 30 times the risk of developing AD by 75 years of age, as compared to those not carrying any E4 allele and this may the case with the general population.

Sirtuin proteins are a class of proteins that possess either mono-ADP-ribosyltransferase, or deacylase activity, including deacetylase, desuccinylase, demalonylase, demyristoylase and depalmitoylase activity (see, e.g., http://en.wikipedia.org/wiki/Sirtuins—cite_note-pmid15128440-2 IRT1) and deacetylates and coactivates the retinoic acid receptor beta that upregulates the expression of alpha-secretase (ADAM10).

Unless otherwise indicated, reference to a compound (e.g., to alaproclate and other “related” compounds as described herein) should be construed broadly to include pharmaceutically acceptable salts, prodrugs, tautomers, alternate solid forms, non-covalent complexes, and combinations thereof, of a chemical entity of the depicted structure or chemical name.

Generally, reference to a certain element such as hydrogen or H is meant to include all isotopes of that element. For example, if an R group is defined to include hydrogen or H, it also includes deuterium and tritium. Accordingly, isotopically labeled compounds are within the scope of this invention.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counterions. Salts can form from or incorporate one or more deprotonated acidic groups (e.g. carboxylic acids), one or more protonated basic groups (e.g. amines), or both (e.g. zwitterions).

A prodrug is a compound that is converted to a therapeutically active compound after administration. For example, conversion may occur by hydrolysis of an ester group, such as a C₁-C₆ alkyl ester of the carboxylic acid group of the present compounds, or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in equilibrium with one another. For example, tautomers may be related by transfer of a proton, hydrogen atom, or hydride ion.

Unless stereochemistry is explicitly depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like. In various embodiments alternate solid forms of any of the compounds described herein are contemplated.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN), and the like.

The term “alkyl” refers to and covers any and all groups that are known as normal alkyl, branched-chain alkyl, cycloalkyl and also cycloalkyl-alkyl. Illustrative alkyl groups include, but are not limited to methyl, ethyl, n-propyl, isopropyl, n-butyl, isobutyl, sec-butyl, t-butyl, octyl, and decyl. The term “cycloalkyl” refers to cyclic, including polycyclic, saturated hydrocarbyl groups. Examples include, but are not limited to cyclopentyl, cyclohexyl, dicyclopentyl, norbornyl, octahydronapthyl, and spiro[3.4]octyl. In certain embodiments, alkyl groups contain 1-12 carbon atoms (C1-12 alkyl), or 1-9 carbon atoms (C₁₋₉ alkyl), or 1-6 carbon atoms (C₁₋₆ alkyl), or 1-5 carbon atoms (C₁₋₅ alkyl), or carbon atoms (C₁₋₄ alkyl), or 1-3 carbon atoms (C₁₋₃ alkyl), or 1-2 carbon atoms (C₁₋₂ alkyl).

By way of example, the term “C₁₋₆ alkyl group” refers to a straight chain or branched chain alkyl group having 1 to 6 carbon atoms, and may be exemplified by a methyl group, an ethyl group, an n-propyl group, an isopropyl group, an n-butyl group, an isobutyl group, a tert-butyl group, a sec-butyl group, an n-pentyl group, a tert-amyl group, a 3-methylbutyl group, a neopentyl group, and an n-hexyl group.

The term “alkoxy” as used herein means an alkyl group bound through a single, terminal oxygen atom. An “alkoxy” group may be represented as —O-alkyl where alkyl is as defined above. The term “aryloxy” is used in a similar fashion, and may be represented as —O-aryl, with aryl as defined below. The term “hydroxy” refers to —OH.

Similarly, the term “alkylthio” as used herein means an alkyl group bound through a single, terminal sulfur atom. An “alkylthio” group may be represented as —S-alkyl where alkyl is as defined above. The term “arylthio” is used similarly, and may be represented as —S-aryl, with aryl as defined below. The term “mercapto” refers to —SH.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

The term “heteroaryl group” refers to a monocyclic or condensed-ring aromatic heterocyclic group containing one or more hetero-atoms selected from O, S and N. If the aromatic heterocyclic group has a condensed ring, it can include a partially hydrogenated monocyclic group. Examples of such a heteroaryl group include a pyrazolyl group, a thiazolyl group, an isothiazolyl group, a thiadiazolyl group, an imidazolyl group, a furyl group, a thienyl group, an oxazolyl group, an isoxazolyl group, a pyrrolyl group, an imidazolyl group, a (1,2,3)- and (1,2,4)-triazolyl group, a tetrazolyl group, a pyranyl group, a pyridyl group, a pyrimidinyl group, a pyrazinyl group, a pyridazinyl group, a quinolyl group, an isoquinolyl group, a benzofuranyl group, an isobenzofuranyl group, an indolyl group, an isoindolyl group, an indazolyl group, a benzoimidazolyl group, a benzotriazolyl group, a benzoxazolyl group, a benzothiazolyl group, a benzo[b]thiophenyl group, a thieno[2,3-b]thiophenyl group, a (1,2)- and (1,3)-benzoxathiol group, a chromenyl group, a 2-oxochromenyl group, a benzothiadiazolyl group, a quinolizinyl group, a phthalazinyl group, a naphthyridinyl group, a quinoxalinyl group, a quinazolinyl group, a cinnolinyl group, and a carbazolyl group.

A “derivative” of a compound means a chemically modified compound wherein the chemical modification takes place at one or more functional groups of the compound. The derivative however, is expected to retain, or enhance, the pharmacological activity of the compound from which it is derived.

As used herein, “administering” refers to local and systemic administration, e.g., including enteral, parenteral, pulmonary, and topical/transdermal administration. Routes of administration for agents (e.g., alaproclate and other “related” compounds as described herein, or a tautomer(s) or stereoisomer(s) thereof, or pharmaceutically acceptable salts or solvates of said alaproclate and other “related” compounds, said stereoisomer(s), or said tautomer(s), or analogues, derivatives, or prodrugs thereof) that find use in the methods described herein include, e.g., oral (per os (p.o.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering the agent(s) described herein or composition to a mammal so that the agent(s) or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration” or “administering in conjunction with” when used, for example with respect to the active agent(s) described herein e.g., alaproclate and other “related” compounds described herein and a second active agent (e.g., a cognition enhancer), refers to administration of the agent(s) and/the second active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, preferably 30% or 40% or greater, more preferably 50% or 60% or greater, most preferably 70% or 80% or 90% or greater) of their maximum serum concentration for any given dose.

The term “effective amount” or “pharmaceutically effective amount” refer to the amount and/or dosage, and/or dosage regime of one or more agent(s) necessary to bring about the desired result e.g., an amount sufficient to increase expression of SirT1 and/or ADAM10 in a mammal, and/or to mitigate in a mammal one or more symptoms associated with mild cognitive impairment (MCI), or an amount sufficient to lessen the severity or delay the progression of a disease characterized by amyloid deposits in the brain in a mammal (e.g., therapeutically effective amounts), an amount sufficient to reduce the risk or delaying the onset, and/or reduce the ultimate severity of a disease characterized by amyloid deposits in the brain in a mammal (e.g., prophylactically effective amounts).

The phrase “cause to be administered” refers to the actions taken by a medical professional (e.g., a physician), or a person controlling medical care of a subject, that control and/or permit the administration of the agent(s) at issue to the subject. Causing to be administered can involve diagnosis and/or determination of an appropriate therapeutic or prophylactic regimen, and/or prescribing particular agent(s) for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, but is not limited to, reduction or elimination of one or more markers that are characteristic of the pathology or disease (e.g., of total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ42/Aβ40 ratio, Aβ42/Aβ38 ratio, sAPPα, sAPPα/sAPPβ ratio, sAPPα/Aβ40 ratio, sAPPα/Aβ42 ratio, etc.) and/or reduction, stabilization or reversal of one or more diagnostic criteria (e.g., clinical dementia rating (CDR)).

As used herein, the phrase “consisting essentially of” refers to the genera or species of active pharmaceutical agents recited in a method or composition, and further can include other agents that, on their own do not substantial activity for the recited indication or purpose. In some embodiments, the phrase “consisting essentially of” expressly excludes the inclusion of one or more additional agents that have neuropharmacological activity other than the recited agent(s) (e.g., other than alaproclate and other alaproclate “related” active agents described herein). In some embodiments, the phrase “consisting essentially of” expressly excludes the inclusion of one or more additional active agents other than the active agent(s) described herein (e.g., other than alaproclate and other alaproclate “related” active agents described herein). In some embodiments, the phrase “consisting essentially of” expressly excludes the inclusion of one or more acetylcholinesterase inhibitors.

The terms “subject”, “individual”, and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments the subject may not be under the care or prescription of a physician or other health worker.

The term “formulation” or “drug formulation” or “dosage form” or “pharmaceutical formulation” as used herein refers to a composition containing at least one therapeutic agent or medication for delivery to a subject. In certain embodiments the dosage form comprises a given “formulation” or “drug formulation” and may be administered to a patient in the form of a lozenge, pill, tablet, capsule, suppository, membrane, strip, liquid, patch, film, gel, spray or other form.

The term “mucosal membrane” refers generally to any of the mucus-coated biological membranes in the body. In certain embodiments active agent(s) described herein can be administered herein via any mucous membrane found in the body, including, but not limited to buccal, perlingual, nasal, sublingual, pulmonary, rectal, and vaginal mucosa. Absorption through the mucosal membranes of the oral cavity and those of the gut are of interest. Thus, peroral, buccal, sublingual, gingival and palatal absorption are contemplated herein.

The term “transmucosal” delivery of a drug and the like is meant to encompass all forms of delivery across or through a mucosal membrane.

The term “bioadhesion” as used herein refers to the process of adhesion of the dosage form(s) to a biological surface, e.g., mucosal membranes.

“Controlled drug delivery” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

“Sustained drug delivery” refers to release or administration of a drug from a source (e.g., a drug formulation) in a sustained fashion over a protracted yet specific period of time, that may extend from several minutes to a few hours, days, weeks or months. In various embodiments the term “sustained” will be used to refer to delivery of consistent and/or substantially constant levels of drug over a time period ranging from a few minutes to a day, with a profile characterized by the absence of an immediate release phase, such as the one obtained from IV administration.

The term “Tₘₐₓ” as used herein means the time point of maximum observed plasma concentration.

The term “Cₘₐₓ” as used herein means the maximum observed plasma concentration.

The term “plasma t_{1/2}” as used herein means the observed “plasma half-life” and represents the time required for the drug plasma concentration to reach the 50% of its maximal value (Cₘₐₓ). This facilitates determination of the mean duration of pharmacological effects. In addition, it facilitates direct and meaningful comparisons of the duration of different test articles after delivery via the same or different routes.

The term “Optimal Therapeutic Targeting Ratio” or “OTTR” represents the average time that the drug is present at therapeutic levels, defined as time within which the drug plasma concentration is maintained above 50% of Cₘₐₓ normalized by the drug's elimination half-life multiplied by the ratio of the Cₘₐₓ obtained in the dosage form of interest over the Cₘₐₓ following IV administration of equivalent doses and it is calculated by the formula:

OTTR=(C^{IV}ₘₐₓ/Cₘₐₓ)×(Dose/Dose^{IV})(Time above 50% of Cₘₐₓ)/(Terminal^{IV} elimination half-life of the drug).

The term “substantially pure” means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical or chemical properties, of the compound. Methods for purification of the compounds to produce substantially chemically pure compounds are known to those of skill in the art. A substantially chemically pure compound may, however, be a mixture of stereoisomers or isomers. In such instances, further purification might increase the specific activity of the compound.

The term “substantially pure” when used with respect to enantiomers indicates that one particular enantiomer (e.g. an S enantiomer or an R enantiomer) is substantially free of its stereoisomer. In various embodiments substantially pure indicates that a particular enantiomer is at least 70%, or at least 80%, or at least 90%, or at least 95%, or at least 98%, or at least 99% of the purified compound. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds of the can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

### BRIEF DESCRIPTION OF THE DRAWINGS

FIG. 1 illustrates that alternative cleavage of APP produces either pro-AD peptides (sAPPβ, Aβ, Jcasp, C31) that mediate synaptic loss, neurite retraction and ultimately programmed cell death; or anti-AD peptides (sAPPα, and αCTF) that mediate synaptic maintenance and inhibit programmed cell death (Moi (2009) Neurodegen. 4: 27).

FIG. 2 illustrates signal transduction pathways associated with SirT1 (Bonda et al. (2011) Lancet Neurol., 10: 275-279).

FIG. 3 shows SIRT1 levels in human serum (Mishra et al. (2013) PLoS One 9(1): e86852.

FIG. 4, panels A-D, show that ApoE co-precipitates with APP. Surface plasmon resonance analysis of the binding of trxeAPP290-624 to trx-ApoE4 (panel A) and trx-ApoE3 (panel B). Panel C: Following transfection of A172 cells with ApoE3 or E4, lysate was immunoprecipitated using an N-terminal anti-APP antibody followed by SDS/PAGE and WB to detect FL APP (upper band, “APP”), sAPPα (lower band, “APP”) and ApoE. Panel D: A similar experiment was performed using H4 cells and IP with a c-terminal anti-APP antibody to detect FL APP (APP) and ApoE. GAPDH was used as a pre-IP loading control for both.

FIG. 5, panels A-C, shows that ApoE4, but not ApoE3, significantly reduces sAPPα and lowers a/p ratio. A172 cells were transfected with ApoE isoforms and 24 hours later, sAPPα (A) in the medium was determined using AlphaLISA immunoassay (Perkin Elmer) according to the manufacturer's instructions with modification (Theendakara et al. (2013) Proc. Natl. Acad. Sci. USA, 110(45):18303-18308). Api-42 (panel B) in the cell extracts was assayed using an ELISA (Life Technologies). Panel C: sAPPα/Aβ1-42 ratios were calculated using the raw data from each. Data (mean±SE) are from four experiments performed in triplicate, *P<0.05.

FIG. 6, panels A-C: Panel A: In cells transfected with ApoE4(+), both APP (p-APPThr668) and tau (p-tau409) phosphorylation are increased. The GSK3j8 inhibitor, CHIR, restores sAPPα levels (panel B) and reduces APP phosphorylation (panel C) in the presence of ApoE4.

FIG. 7, panels A-D, shows that expression of ApoE4 after transfection increases SirT2 (panels A, B) and decreases SirT1 (panels A, C) levels, greatly increasing the SirT2/SirT1 ratio (panel D).

FIG. 8, panels A-C, show SirT levels in AD postmortem tissue. Panel A: Representative immunoblots of temporoparietal homogenates from normal and AD patients probed for SirT1, T2, and T6 show that SirT2 (panel B) is unchanged, but SirT1 (panel C) is decreased.

FIG. 9, panels A and B show that overexpression of SirT1 reverses ApoE4-mediated reduction in sAPPα. Panel A: Following transfection of A172 cells with ApoE4 and SirT1 at 1:1 and 1:2, sAPPα in the medium determined by AlphaLISA (Perkin-Elmer) was shown to decrease with ApoE4, but was restored by co-expression with SirT1. Panel B: Similar findings were seen with cell extracts immunoprecipitated with an N-terminal anti-APP antibody followed by SDS/PAGE and WB to detect sAPPα.

FIGS. 10A-10E illustrate SirT1 dose-response with A02 and A03. FIG. 10A: In N2A neuroblastoma cells stably transfected with ApoE4, A03—but not A02—generated a SirT1 dose-response. FIG. 10B: The molecular structure of A03 is shown. mRNA levels of SirT1 (FIG. 10C) and ADAM10 (FIG. 10D) were normalized in A172 cells transfected with ApoE4 upon treatment with A03 at 2 μM. FIG. 10E: A03 shows excellent brain penetration after subcutaneous injection in mice.

FIG. 11 shows an illustrative AlphaLISA assay developed using commercial antibodies to the N- & C-terminal regions of SirT1. In certain embodiments ApoE3 cells are used as a control and drug treatment can be done on ApoE4 cells.

FIG. 12 shows an illustrative, in-cell ELISA protocol for determination of selectivity.

FIG. 13A illustrates selection criteria for validated “hits” and FIG. 13B illustrate a workflow and selection criteria.

FIG. 14 shows illustrative, but non-limiting, modifications of alaproclate.

FIG. 15 illustrative synthesis schemes.

### DETAILED DESCRIPTION

In various embodiments, new approaches to the treatment and/or prophylaxis of Mild Cognitive Impairment (MCI) and Alzheimer's disease (AD), new therapeutic agents and modalities, and method of identifying new therapeutic agents are provided.

In particular, in various embodiments, drugs and drug candidates that reverse ApoE4-mediated decreases in SirT1 levels as well as increase pro-AD processing of APP and memory loss are identified. New drugs and methods for drug discovery are provided that that not only influence brain aging but also the onset or progression of the sporadic form of AD.

The studies described herein link, for the first time ApoE4, the major risk factor for sporadic Alzheimer's disease, with the sirtuins, key longevity determinants. Additionally, the drug alaproclate (also known as 1-(4-chlorophenyl)-2-methylpropan-2-yl 2-aminopropanoate or GEA-654) as the first candidate therapeutic that targets this new link. Our data shows that while both ApoE3 and ApoE4 associate with amyloid precursor protein (APP) at nanomolar affinities, only ApoE4 significantly modulates amyloid precursor protein (APP) processing and function. ApoE4 causes increased pro-AD cleavage at the β, γ, and caspase sites resulting in the production of four pro-AD-peptides (sAPPβ, Aβ, Jcasp, and C31) that mediate neurite retraction, synaptic reorganization, and ultimately neuronal loss, while suppressing the cleavage at the α-site that produces the trophic peptide sAPPα and the inhibitor of APP γ-site cleavage, αCTF (Bredesen (2009) Mol. Neurodegener. 4: 27; Bredesen and John (2013) EMBO Mol. Med. 5(6):795-798). ApoE4 expression is also associated with increased APP-Thr668 phosphorylation (p-APP) and tau phosphorylation (p-Tau). Thus ApoE4 enhances the AD phenotype and increases the ratio of the above mentioned pro-AD relative to anti-AD peptides (Bredesen (2009) Mol. Neurodegener. 4: 27; Bredesen and John (2013) EMBO Mol. Med. 5(6):795-798; Theendakara et al. (2013) Proc. Natl. Acad. Sci. USA, 10(45):18303-18308).

The ApoE4 allele (chromosomal locus 19q13) is the single most important genetic risk factor associated with AD. This allele confers increased risk for sporadic and late-onset AD (LOAD). Despite it being known for over a decade that the ApoE4 allele is somehow contributory to the disease process, the precise molecular mechanisms underlying ApoE and APP interactions, direct or indirect, that result in ApoE4-mediated toxicity remained unclear. The studies provided herein shed light on ApoE4-mediated toxicity and revealed SirT1 as a key mediator that is differentially affected by ApoE4 vs. ApoE3, and show that ApoE4 triggers a marked reduction in the ratio of SirT1/SirT2.

These data offer new insights into why the ApoE4 allele is the major risk factor or susceptibility gene associated with AD, and therefore represents an excellent target for AD drug discovery. Overexpression of SirT1 in ApoE4 cellular model increases sAPPα levels, while overexpression in an AD transgenic model decreases the amyloid pathology (Donmez et al. (2010) Cell, 142(2):320-332). Interestingly, recent reports show significant decrease in serum SirT1 levels in MCI and AD patients and changes in plasma inflammatory mediators in ApoE4 carriers (Kumar et al. (2013) PLoS One 8(4):e61560; Ringman et al. (2012) Arch. Neurol. 69(6): 757-764). The mechanism identified herein integrates multiple factors and pathways inter-connected via ApoE4 underlying AD.

The data presented herein link, for the first time ApoE4, the major risk factor for Alzheimer's disease, with the Sirtuins, major longevity determinants.

The results described herein are consistent with the idea that ApoE4-mediated signaling affects an endogenous program that mediates synaptic plasticity balance. The data shown herein offer new insight into why the ApoE4 allele is the major susceptibility gene associated with AD, and should therefore be considered a critical target for AD drug discovery.

In particular, the data indicate that ApoE4, p-APP, p-tau, and SirT1 all appear to be part of a signaling network that is altered in AD. The studies presented herein do not simply focus on a single variable, but take a systems approach and integrate multiple factors and pathways all inter-connected via ApoE4 within the network imbalance that underlies AD. We recently showed that, while both ApoE3 and ApoE4 associate with APP, ApoE4 associates with nanomolar affinities (Kd ˜80 nM), and only ApoE4 significantly:
(a) reduced the ratio of sAPPα to Aβ;
(b) reduced SirT1 expression, resulting in a markedly reduction of SirT1 levels and in the ratio of neuroprotective SirT1 to neurotoxic SirT2; and
(c) triggered tau and APP phosphorylation; and (d) induced programmed cell death (see Example 1).

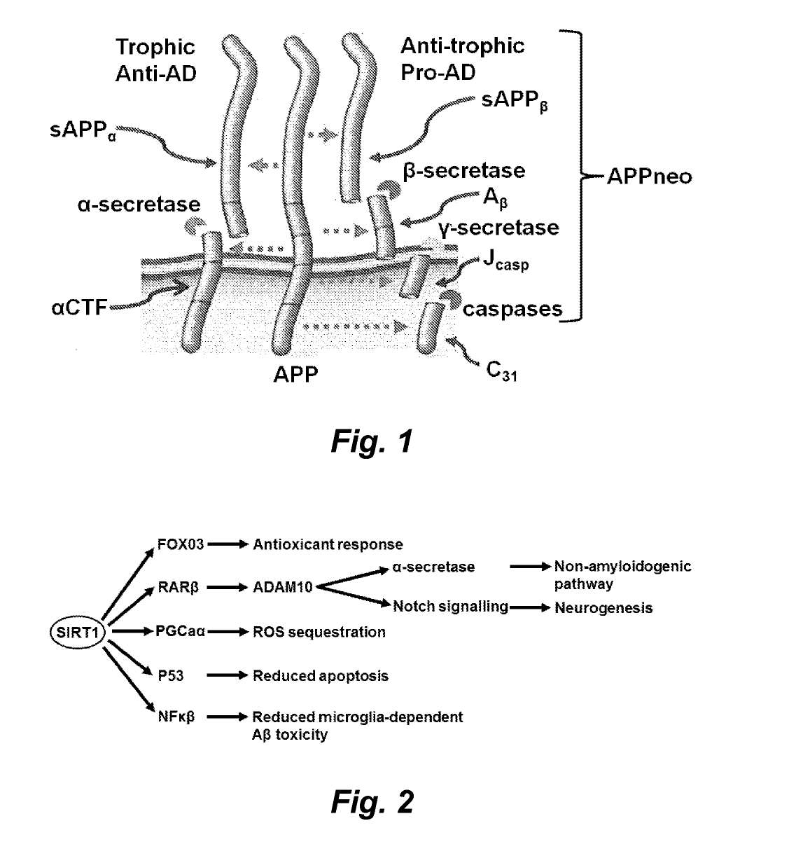

Our studies also show that ApoE4 mediates pro-AD effects that include a shift of APP processing in favor of the pro-AD peptides Aβ, sAPPβ, Jcasp, and C31 (see FIG. 1), with an alteration in downstream signaling, and this shift may be a critical determinant of the ApoE4-associated risk for Alzheimer's disease. The data thus explain why the ApoE4 allele is the major susceptibility gene associated with AD and should therefore be considered a critical target for AD drug discovery. In particular, the studies described herein identified the sirtuin SirT1 as a mediator that is differentially affected by ApoE4 vs. ApoE3, and show that ApoE4 triggers a marked reduction in the ratio of SirT1/SirT2.

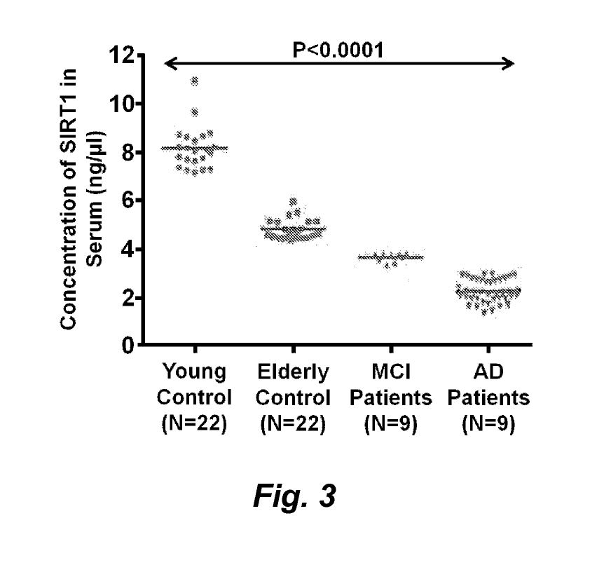

Sirtuins are NAD-dependent deacetylases that influence aging and have a wide spectrum of metabolic and stress-tolerance functions (Sinclair (2005) Mech. Ageing Dev. 126(9):987-1002). Of the seven mammalian sirtuins, the SIR2 ortholog SirT1 is a nuclear enzyme that deacetylates numerous regulatory proteins and genes, such as in the antioxidant response (FOXO3) (Brunet et al. (2004) Science, 303(5666):2011-2015; Elliott et al. 92008) Curr. Opin. Investig. Drugs, 9(4):371-378); anti-inflammatory response (NFκB) (Yeung et al. (2014) EMBO J. 23(12):2369-2380), anti-apoptotic response (p53) (Langley et al. (2002) EMBO J. 21(10): 2383-2396), and mitochondrial biogenesis and ROS sequestration (PGC1α) (Wang et al. (2010) Biochim. Biophys. Acta. 1804(8):1690-1694) to trigger resistance to metabolic, oxidative, heat, and hypoxic stress (Bonda et al. (2011) Lancet Neurol. 10(3): 275-279; Guarente (2009) Science, 324(5932):1281-1282) (FIG. 2). SirT1 has been directly implicated in neuronal protection against stress in cultured cells (Qin et al. (2006) J. Biol. Chem. 281(31): 21745-21754). In mice, SirT1 has been shown to protect against neurodegeneration in the p25 overexpression model (Kim et al. (2007) EMBO J. 26(13): 3169-3179), as well as in Wallerian degeneration slow mice (Araki and Milbrandt (2004) Science, 305(5686): 1010-1013). More generally, SirT1 mediates at least some of the effects of calorie restriction (CR) (Guarente (2008) Cell, 132(2): 171-176), and CR has been shown to lead to attenuation of AP deposition in an AD model (Patel et al. (2005) Neurobiol. Aging. 26(7): 995-1000). Activation of SirT1 results in a robust protective cellular response such as activation of the retinoic acid receptor-β (RARβ) protein, by SirT1 removal an acetyl group from a lysine residue and increases in transcription of the ADAM10 gene and therefore α-secretase production (Tippmann et al. (2009) FASEB J. 23(6): 1643-1654). In addition to being the first cleavage enzyme in the trophic APP processing pathway, ADAM10 also initiates the Notch signaling pathway by cleavage of Notch 1 after binding of Notch to the membrane-bound Notch receptor (Costa et al. (2005) Trends Neurosci. 28(8):429-435). This, followed by γ-secretase cleavage, liberates the intracellular Notch domain (NICD) that forms a transcription complex and upregulates the transcription of genes involved in neurogenesis (Costa et al. (2005) Trends Neurosci. 28(8):429-435; Xiao et al. (2012) Int. J. Physiol., Pathophysiol. & Pharmacol., 1(2):192-202).

Overexpression of SirT1 is also reported to prevent microglia-dependent AP toxicity (Heneka et al. (2010) J. Neural Transm. 117(8): 919-947) through inhibition of NFκB signaling (Chen et al. (2005) J. Biol. Chem. 280(48): 40364-40374). SirT1 deacetylates the lysine 310 residue of the RelA/p65 subunit of NFκB, thereby preventing its transcriptional activity (Yang and Chen (2010) Mol. Cell Biol. 30(9): 2170-2180). SirT1 levels in brain were shown to affect AP plaque formation, amyloid pathology, and cognitive decline in an AD mouse model (APPswe/PSEN1dE9 double transgenic) (Donmez et al. (2010) Cell, 142(2):320-332). The induction of brain pathology and behavioral deficits was mitigated in AD mice overexpressing SirT1 in brain, and was exacerbated in SirT1 knock-outs (Id.).

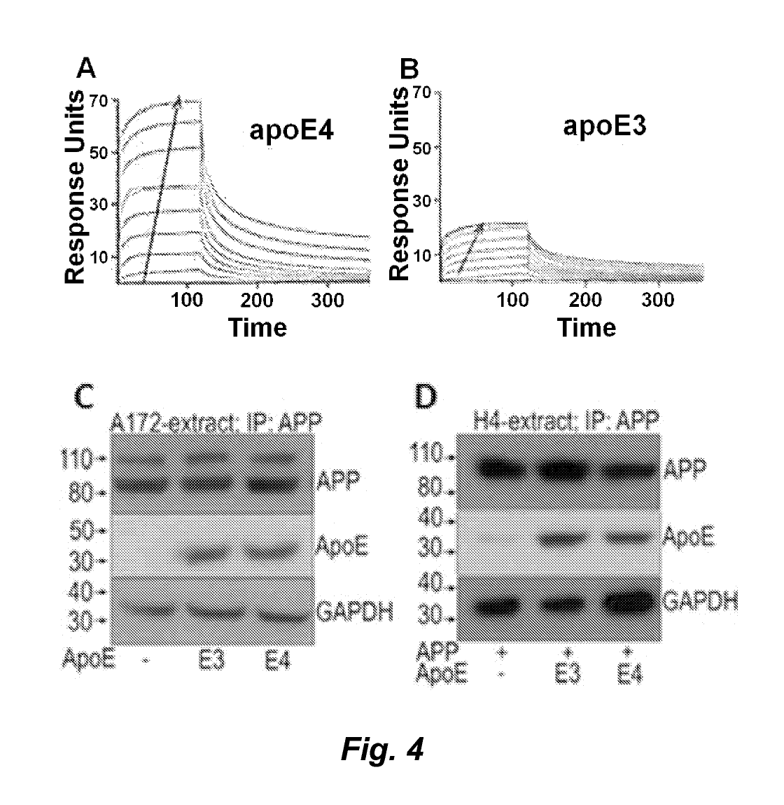

A significant decrease in SirT1 levels in parietal cortex in AD-patient autopsy specimens has been reported, and these decreases showed good correlation to duration of symptoms and tau accumulation (Julien et al. (2009) J. Neuropathol. Exp. Neurol. 68(1): 48-58). The exact relationship between SirT1 and AD pathology and the sequence of events has remained unclear. In a first-of-a-kind study, Kumar et al. (2013) PLoS One 8(4):e61560, revealed a decline in serum concentration of SirT1 in even healthy individuals as they age (FIG. 3). In patients with AD and MCI the decline was even more pronounced, which provides an opportunity to develop this protein as a predictive biomarker of AD in early stages.

Our therapeutic approach targets ApoE4 effects on SirT1, and reverses the SirT1 deficit and the “sirtuinversion” (alteration of the SirT1:SirT2 ratio).

In vitro assays described herein provide a simple, medium-throughput or high-throughput system for the screening and identification of program mediators and therapeutic candidates that interrupt and/or alter the APP-ApoE4 interaction and return the parameters noted above to normal. This system was used to identify the first such candidate therapeutic alaproclate (also known as 1-(4-chlorophenyl)-2-methylpropan-2-yl 2-aminopropanoate or GEA-654) (see Examples) and related compounds as described herein. Thus, a set of drug candidates was identified that interrupt the APP-ApoE interaction and returned the parameters noted above to normal. The data presented herein with respect to the use of these agents support the hypothesis that neuronal connectivity, influenced by the ratios of critical mediators including sAPPα:Aβ, SirT1:SirT2, APP:p-APP, and tau:p-tau, is programmatically altered by ApoE4.

Thus, in addition to alaproclate related compounds are identified that are believed to normalize ApoE4-modulated effects on SirT1, SirT1/SirT2 ratios, and APP processing, thus impacting both disease risk and progression.

It is believed the compounds described herein are the first identified to target the ApoE4-induced decrease in SirT1 and sAPPα levels in the brain. Given the current clinical landscape, it is likely that therapeutics targeting Aβ or tau alone will not address all of the pathogenic events in the disease. It is believed that targeting the ApoE4 risk factor and underlying mechanisms as described herein can provide an effective treatment that potentially could be used by itself or in combination current treatments in development for AD.

Accordingly in certain embodiments, methods of increasing the expression of SirT1, and/or increasing the expression of ADAM10, and/or increasing sAPPα, and/or decreasing p-tau in a mammal are provided where the methods involve administering to the mammal (e.g., a human or a non-human mammal) alaproclate and/or one or more of the other active agents described herein.

In certain embodiments methods of normalizing ApoE4 mediated effects on SirT1, and/or normalizing SirT1/SirT2 ratios, and/or normalizing APP processing in a mammal, are provided where the methods involve administering to the mammal (e.g., a human or a non-human mammal) alaproclate and/or one or more of the other active agents described herein.

In certain embodiments methods of preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal, are provided where the methods involve administering to the mammal (e.g., a human or a non-human mammal) alaproclate and/or one or more of the other active agents described herein.

In certain embodiments methods of ameliorating one or more symptoms of Alzheimer's disease, and/or reversing Alzheimer's disease, and/or reducing the rate of progression of Alzheimer's disease in a mammal are provided where the methods involve administering to the mammal (e.g., a human or a non-human mammal) alaproclate and/or one or more of the other active agents described herein.

Given the effects of SirT1 upregulation on lifespan, and/or healthspan, and/or diabetes, and/or metabolic disease, in certain embodiments, methods of increasing the lifespan and/or healthspan of a mammal, and/or treating diabetes and/or metabolic disease are provided where the methods involve administering to the mammal (e.g., a human or a non-human mammal) alaproclate and/or one or more of the other active agents described herein.

### Therapeutic and Prophylactic Methods.

In various embodiments therapeutic and/or prophylactic methods are provided that utilize the active agent(s) (e.g., alaproclate (1-(4-chlorophenyl)-2-methylpropan-2-yl 2-aminopropanoate or GEA-654) and other “related” compounds) described herein, or a tautomer(s) or stereoisomer(s) thereof, or pharmaceutically acceptable salts or solvates of said alaproclate and other “related” compounds, said stereoisomer(s), or said tautomer(s), or analogues, derivatives, or prodrugs thereof) are provided. Typically the methods involve administering one or more active agent(s) to a subject (e.g., to a human in need thereof) in an amount sufficient to realize the desired therapeutic or prophylactic result.

### Prophylaxis

In certain embodiments active agent(s) (e.g., alaproclate and other compounds described herein, or a tautomer(s) or stereoisomer(s) thereof, or pharmaceutically acceptable salts or solvates of said compounds, said stereoisomer(s), or said tautomer(s), or analogues, derivatives, or prodrugs thereof) are utilized in various prophylactic contexts. Thus, for example, in certain embodiments, the active agent(s) can be used to prevent or delay the onset of a pre-Alzheimer's cognitive dysfunction, and/or to ameliorate one more symptoms of a pre-Alzheimer's condition and/or cognitive dysfunction, and/or to prevent or delay the progression of a pre-Alzheimer's condition and/or cognitive dysfunction to Alzheimer's disease.

In certain embodiments, the subjects are cognitively asymptomatic. Additionally or alternatively, in certain embodiments, the subjects are asymptomatic with respect to biomarkers (e.g., p-tau, Aβ, etc.). In certain embodiments, the subjects are fully asymptomatic, but simply possess one copy of the ApoE4 allele. In certain embodiments, the subjects are fully asymptomatic, but simply possess one two copies of the ApoE4 allele.

In certain embodiments, the prophylactic methods described herein are contemplated for subjects identified as “at risk” and/or as having evidence of early Alzheimer's Disease (AD) pathological changes, but who do not meet clinical criteria for MCI or dementia. Without being bound to a particular theory, it is believed that even this “preclinical” stage of the disease represents a continuum from completely asymptomatic individuals with biomarker evidence suggestive of AD-pathophysiological process(es) (abbreviated as AD-P, see, e.g., Sperling et al. (2011) Alzheimer's & Dementia, 1-13) at risk for progression to AD dementia to biomarker-positive individuals who are already demonstrating very subtle decline but not yet meeting standardized criteria for MCI (see, e.g., Albert et al. (2011) Alzheimer's and Dementia, 1-10 (doi:10.1016/j.jalz.2011.03.008).

This latter group of individuals might be classified as “not normal, not MCI” but would be can be designated “pre-symptomatic” or “pre-clinical or “asymptomatic” or “premanifest”). In various embodiments this continuum of pre-symptomatic AD can also encompass, but is not necessarily limited to, (1) individuals who carry one or more apolipoprotein E (APOE) ε4 alleles who are known or believed to have an increased risk of developing AD dementia, at the point they are AD-P biomarker-positive, and (2) carriers of autosomal dominant mutations, who are in the presymptomatic biomarker-positive stage of their illness, and who will almost certainly manifest clinical symptoms and progress to dementia.

A biomarker model has been proposed in which the most widely validated biomarkers of AD-P become abnormal and likewise reach a ceiling in an ordered manner (see, e.g., Jack et al. (2010) Lancet Neurol., 9: 119-128.). This biomarker model parallels proposed pathophysiological sequence of (pre-AD/AD), and is relevant to tracking the preclinical (asymptomatic) stages of AD (see, e.g., FIG. 3 in Sperling et al. (2011) Alzheimer's & Dementia, 1-13). Biomarkers of brain amyloidosis include, but are not limited to reductions in CSF Aβ₄₂ and increased amyloid tracer retention on positron emission tomography (PET) imaging. Elevated CSF tau is not specific to AD and is thought to be a biomarker of neuronal injury. Decreased fluorodeoxyglucose 18F (FDG) uptake on PET with a temporoparietal pattern of hypometabolism is a biomarker of AD-related synaptic dysfunction. Brain atrophy on structural magnetic resonance imaging (MRI) in a characteristic pattern involving the medial temporal lobes, paralimbic and temporoparietal cortices is a biomarker of AD-related neurodegeneration. Other markers include, but are not limited to volumetric MRI, FDG-PET, or plasma biomarkers (see, e.g., Vemuri et al. (2009) Neurology, 73: 294-301; Yaffe et al. (2011) JAMA 305: 261-266).

In certain embodiments the subjects suitable for the prophylactic methods contemplated herein include, but are not limited to, subjects characterized as having asymptomatic cerebral amyloidosis. In various embodiments these individuals have biomarker evidence of Aβ accumulation with elevated tracer retention on PET amyloid imaging and/or low Aβ42 in CSF assay, but typically no detectable evidence of additional brain alterations suggestive of neurodegeneration or subtle cognitive and/or behavioral symptomatology.

It is noted that currently available CSF and PET imaging biomarkers of Aβ primarily provide evidence of amyloid accumulation and deposition of fibrillar forms of amyloid. Data suggest that soluble or oligomeric forms of Aβ are likely in equilibrium with plaques, which may serve as reservoirs. In certain embodiments it is contemplated that there is an identifiable preplaque stage in which only soluble forms of Aβ are present. In certain embodiments it is contemplated that oligomeric forms of amyloid may be critical in the pathological cascade, and provide useful markers. In addition, early synaptic changes may be present before evidence of amyloid accumulation.

In certain embodiments the subjects suitable for the prophylactic methods contemplated herein include, but are not limited to, subjects characterized as amyloid positive with evidence of synaptic dysfunction and/or early neurodegeneration. In various embodiments these subjects have evidence of amyloid positivity and presence of one or more markers of “downstream” AD-related neuronal injury. Illustrative, but non-limiting markers of neuronal injury include, but are not limited to (1) elevated CSF tau or phospho-tau, (2) hypometabolism in an AD-like pattern (i.e., posterior cingulate, precuneus, and/or temporoparietal cortices) on FDG-PET, and (3) cortical thinning/gray matter loss in a specific anatomic distribution (i.e., lateral and medial parietal, posterior cingulate, and lateral temporal cortices) and/or hippocampal atrophy on volumetric MRI. Other markers include, but are not limited to fMRI measures of default network connectivity. In certain embodiments early synaptic dysfunction, as assessed by functional imaging techniques such as FDG-PET and fMRI, can be detectable before volumetric loss. Without being bound to a particular theory, it is believed that amyloid-positive individuals with evidence of early neurodegeneration may be farther down the trajectory (i.e., in later stages of preclinical (asymptomatic) AD).

In certain embodiments the subjects suitable for the prophylactic methods contemplated herein include, but are not limited to, subjects characterized as amyloid positive with evidence of neurodegeneration and subtle cognitive decline. Without being bound to a particular theory, it is believed that those individuals with biomarker evidence of amyloid accumulation, early neurodegeneration, and evidence of subtle cognitive decline are in the last stage of preclinical (asymptomatic) AD, and are approaching the border zone with clinical criteria for mild cognitive impairment (MCI). These individuals may demonstrate evidence of decline from their own baseline (particularly if proxies of cognitive reserve are taken into consideration), even if they still perform within the “normal” range on standard cognitive measures. Without being bound to a particular theory, it is believed that more sensitive cognitive measures, particularly with challenging episodic memory measures, may detect very subtle cognitive impairment in amyloid-positive individuals. In certain embodiments criteria include, but are not limited to, self-complaint of memory decline or other subtle neurobehavioral changes.

As indicated above, subjects/patients amenable to prophylactic methods described herein include individuals at risk of disease (e.g., a pathology characterized by amyloid plaque formation such as MCI) but not showing symptoms, as well as subjects presently showing certain symptoms or markers. It is known that the risk of MCI and later Alzheimer's disease generally increases with age. Accordingly, in asymptomatic subjects with no other known risk factors, in certain embodiments, prophylactic application is contemplated for subjects over 50 years of age, or subjects over 55 years of age, or subjects over 60 years of age, or subjects over 65 years of age, or subjects over 70 years of age, or subjects over 75 years of age, or subjects over 80 years of age, in particular to prevent or slow the onset or ultimate severity of mild cognitive impairment (MCI), and/or to slow or prevent the progression from MCI to early stage Alzheimer's disease (AD).

In certain embodiments, the methods described herein are especially useful for individuals who do have a known genetic risk of Alzheimer's disease (or other amyloidogenic pathologies), whether they are asymptomatic or showing symptoms of disease. Such individuals include those having relatives who have experienced MCI or AD (e.g., a parent, a grandparent, a sibling), and those whose risk is determined by analysis of genetic or biochemical markers. Genetic markers of risk toward Alzheimer's disease include, for example, mutations in the APP gene, particularly mutations at position 717 and positions 670 and 671 referred to as the Hardy and Swedish mutations respectively (see Hardy (1997) Trends. Neurosci., 20: 154-159). Other markers of risk include mutations in the presenilin genes (PS1 and PS2), family history of AD, having the familial Alzheimer's disease (FAD) mutation, the APOE ε4 allele, hypercholesterolemia or atherosclerosis. Further susceptibility genes for the development of Alzheimer's disease are reviewed, e.g., in Sleegers, et al. (2010) Trends Genet. 26(2): 84-93.

In some embodiments, the subject is asymptomatic but has familial and/or genetic risk factors for developing MCI or Alzheimer's disease. In asymptomatic patients, treatment can begin at any age (e.g., at about 20, about 30, about 40, about 50 years of age). Usually, however, it is not necessary to begin treatment until a patient reaches at least about 40, or at least about 50, or at least about 55, or at least about 60, or at least about 65, or at least about 70 years of age.

In some embodiments, the subject exhibits symptoms, for example, of mild cognitive impairment (MCI) or Alzheimer's disease (AD). Individuals presently suffering from Alzheimer's disease can be recognized from characteristic dementia, as well as the presence of risk factors described above. In addition, a number of diagnostic tests are available for identifying individuals who have AD. These include measurement of CSF Tau, phospho-tau (pTau), Aβ42 levels and C-terminally cleaved APP fragment (APPneo). Elevated total-Tau (tTau), phospho-Tau (pTau), APPneo, soluble Aβ40, pTau/Aβ42 ratio and tTau/Aβ42 ratio, and decreased Aβ42 levels, Aβ42/Aβ40 ratio, Aβ42/Aβ38 ratio, sAPPα levels, sAPPα/sAPPβ ratio, sAPPα/Aβ40 ratio, and sAPPα/Aβ42 ratio signify the presence of AD. In some embodiments, the subject or patient is diagnosed as having MCI. Increased levels of neural thread protein (NTP) in urine and/or increased levels of α2-macroglobulin (α2M) and/or complement factor H (CFH) in plasma are also biomarkers of MCI and/or AD (see, e.g., Anoop et al. (2010) Int. J. Alzheimer's Dis. 2010:606802).

In certain embodiments, subjects amenable to treatment may have age-associated memory impairment (AAMI), or mild cognitive impairment (MCI). The methods described herein are particularly well-suited to the prophylaxis and/or treatment of MCI. In such instances, the methods can delay or prevent the onset of MCI, and or reduce one or more symptoms characteristic of MCI and/or delay or prevent the progression from MCI to early-, mid- or late-stage Alzheimer's disease or reduce the ultimate severity of the disease.

Mild Cognitive Impairment (MCI)

Mild cognitive impairment (MCI, also known as incipient dementia, or isolated memory impairment) is a diagnosis given to individuals who have cognitive impairments beyond that expected for their age and education, but that typically do not interfere significantly with their daily activities (see, e.g., Petersen et al. (1999) Arch. Neurol. 56(3): 303-308). It is considered in many instances to be a boundary or transitional stage between normal aging and dementia. Although MCI can present with a variety of symptoms, when memory loss is the predominant symptom it is termed “amnestic MCI” and is frequently seen as a risk factor for Alzheimer's disease (see, e.g., Grundman et al. (2004) Arch. Neurol. 61(1): 59-66; and on the internet at en.wikipedia.org/wiki/Mild cognitive impairment—cite_note-Grundman-1). When individuals have impairments in domains other than memory it is often classified as non-amnestic single- or multiple-domain MCI and these individuals are believed to be more likely to convert to other dementias (e.g., dementia with Lewy bodies). There is evidence suggesting that while amnestic MCI patients may not meet neuropathologic criteria for Alzheimer's disease, patients may be in a transitional stage of evolving Alzheimer's disease; patients in this hypothesized transitional stage demonstrated diffuse amyloid in the neocortex and frequent neurofibrillary tangles in the medial temporal lobe (see, e.g., Petersen et al. (2006) Arch. Neurol. 63(5): 665-72).

The diagnosis of MCI typically involves a comprehensive clinical assessment including clinical observation, neuroimaging, blood tests and neuropsychological testing. In certain embodiments diagnostic criteria for MIC include, but are not limited to those described by Albert et al. (2011) Alzheimer's & Dementia. 1-10. As described therein, diagnostic criteria include (1) core clinical criteria that could be used by healthcare providers without access to advanced imaging techniques or cerebrospinal fluid analysis, and (2) research criteria that could be used in clinical research settings, including clinical trials. The second set of criteria incorporate the use of biomarkers based on imaging and cerebrospinal fluid measures. The final set of criteria for mild cognitive impairment due to AD has four levels of certainty, depending on the presence and nature of the biomarker findings.

In certain embodiments clinical evaluation/diagnosis of MCI involves: (1) Concern reflecting a change in cognition reported by patient or informant or clinician (i.e., historical or observed evidence of decline over time); (2) Objective evidence of Impairment in one or more cognitive domains, typically including memory (i.e., formal or bedside testing to establish level of cognitive function in multiple domains); (3) Preservation of independence in functional abilities; (4) Not demented; and in certain embodiments, (5) An etiology of MCI consistent with AD pathophysiological processes. Typically vascular, traumatic, and medical causes of cognitive decline, are ruled out where possible. In certain embodiments, when feasible, evidence of longitudinal decline in cognition is identified. Diagnosis is reinforced by a history consistent with AD genetic factors, where relevant.

With respect to impairment in cognitive domain(s), there should be evidence of concern about a change in cognition, in comparison with the person's previous level. There should be evidence of lower performance in one or more cognitive domains that is greater than would be expected for the patient's age and educational background. If repeated assessments are available, then a decline in performance should be evident over time. This change can occur in a variety of cognitive domains, including memory, executive function, attention, language, and visuospatial skills. An impairment in episodic memory (i.e., the ability to learn and retain new information) is seen most commonly in MCI patients who subsequently progress to a diagnosis of AD dementia.

With respect to preservation of independence in functional abilities, it is noted that persons with MCI commonly have mild problems performing complex functional tasks which they used to perform shopping. They may take more time, be less efficient, and make more errors at performing such activities than in the past. Nevertheless, they generally maintain their independence of function in daily life, with minimal aids or assistance.

With respect to dementia, the cognitive changes should be sufficiently mild that there is no evidence of a significant impairment in social or occupational functioning. If an individual has only been evaluated once, change will be inferred from the history and/or evidence that cognitive performance is impaired beyond what would have been expected for that individual.

Cognitive testing is optimal for objectively assessing the degree of cognitive impairment for an individual. Scores on cognitive tests for individuals with MCI are typically 1 to 1.5 standard deviations below the mean for their age and education matched peers on culturally appropriate normative data (i.e., for the impaired domain(s), when available).

Episodic memory (i.e., the ability to learn and retain new information) is most commonly seen in MCI patients who subsequently progress to a diagnosis of AD dementia. There are a variety of episodic memory tests that are useful for identifying those MCI patients who have a high likelihood of progressing to AD dementia within a few years. These tests typically assess both immediate and delayed recall, so that it is possible to determine retention over a delay. Many, although not all, of the tests that have proven useful in this regard are wordlist learning tests with multiple trials. Such tests reveal the rate of learning over time, as well as the maximum amount acquired over the course of the learning trials. They are also useful for demonstrating that the individual is, in fact, paying attention to the task on immediate recall, which then can be used as a baseline to assess the relative amount of material retained on delayed recall. Examples of such tests include (but are not limited to: the Free and Cued Selective Reminding Test, the Rey Auditory Verbal Learning Test, and the California Verbal Learning Test. Other episodic memory measures include, but are not limited to: immediate and delayed recall of a paragraph such as the Logical Memory I and II of the Wechsler Memory Scale Revised (or other versions) and immediate and delayed recall of nonverbal materials, such as the Visual Reproduction subtests of the Wechsler Memory Scale-Revised I and II.

Because other cognitive domains can be impaired among individuals with MCI, it is desirable to examine domains in addition to memory. These include, but are not limited to executive functions (e.g., set-shifting, reasoning, problem-solving, planning), language (e.g., naming, fluency, expressive speech, and comprehension), visuospatial skills, and attentional control (e.g., simple and divided attention). Many clinical neuropsychological measures are available to assess these cognitive domains, including (but not limited to the Trail Making Test (executive function), the Boston Naming Test, letter and category fluency (language), figure copying (spatial skills), and digit span forward (attention).

As indicated above, genetic factors can be incorporated into the diagnosis of MCI. If an autosomal dominant form of AD is known to be present (i.e., mutation in APP, PS1, PS2), then the development of MCI is most likely the precursor to AD dementia. The large majority of these cases develop early onset AD (i.e., onset below 65 years of age).

In addition, there are genetic influences on the development of late onset AD dementia. For example, the presence of one or two ε4 alleles in the apolipoprotein E (APOE) gene is a genetic variant broadly accepted as increasing risk for late-onset AD dementia. Evidence suggests that an individual who meets the clinical, cognitive, and etiologic criteria for MCI, and is also APOE ε4 positive, is more likely to progress to AD dementia within a few years than an individual without this genetic characteristic. It is believed that additional genes play an important, but smaller role than APOE and also confer changes in risk for progression to AD dementia (see, e.g., Bertram et al. (2010) Neuron, 21: 270-281).

In certain embodiments subjects suitable for the prophylactic methods described herein include, but need not be limited to, subjects identified having one or more of the core clinical criteria described above and/or subjects identified with one or more “research criteria” for MCI, e.g., as described below.

“Research criteria” for the identification/prognosis of MCI include, but are not limited to biomarkers that increase the likelihood that MCI syndrome is due to the pathophysiological processes of AD. Without being bound to a particular theory, it is believed that the conjoint application of clinical criteria and biomarkers can result in various levels of certainty that the MCI syndrome is due to AD pathophysiological processes. In certain embodiments, two categories of biomarkers have been the most studied and applied to clinical outcomes are contemplated. These include “Aβ” (which includes CSF Aβ₄₂ and/or PET amyloid imaging) and “biomarkers of neuronal injury” (which include, but are not limited to CSF tau/p-tau, hippocampal, or medial temporal lobe atrophy on MRI, and temporoparietal/precuneus hypometabolism or hypoperfusion on PET or SPECT).

Without being bound to a particular theory, it is believed that evidence of both Aβ, and neuronal injury (either an increase in tau/p-tau or imaging biomarkers in a topographical pattern characteristic of AD), together confers the highest probability that the AD pathophysiological process is present. Conversely, if these biomarkers are negative, this may provide information concerning the likelihood of an alternate diagnosis. It is recognized that biomarker findings may be contradictory and accordingly any biomarker combination is indicative (an indicator) used on the context of a differential diagnosis and not itself dispositive. It is recognized that varying severities of an abnormality may confer different likelihoods or prognoses, that are difficult to quantify accurately for broad application.

For those potential MCI subjects whose clinical and cognitive MCI syndrome is consistent with AD as the etiology, the addition of biomarker analysis effects levels of certainty in the diagnosis. In the most typical example in which the clinical and cognitive syndrome of MCI has been established, including evidence of an episodic memory disorder and a presumed degenerative etiology, the most likely cause is the neurodegenerative process of AD. However, the eventual outcome still has variable degrees of certainty. The likelihood of progression to AD dementia will vary with the severity of the cognitive decline and the nature of the evidence suggesting that AD pathophysiology is the underlying cause. Without being bound to a particular theory it is believed that positive biomarkers reflecting neuronal injury increase the likelihood that progression to dementia will occur within a few years and that positive findings reflecting both Aβ accumulation and neuronal injury together confer the highest likelihood that the diagnosis is MCI due to AD.

A positive Aβ biomarker and a positive biomarker of neuronal injury provide an indication that the MCI syndrome is due to AD processes and the subject is well suited for the methods described herein.

A positive Aβ biomarker in a situation in which neuronal injury biomarkers have not been or cannot be tested or a positive biomarker of neuronal injury in a situation in which Aβ biomarkers have not been or cannot be tested indicate an intermediate likelihood that the MCI syndrome is due to AD. Such subjects are believed to be is well suited for the methods described herein

Negative biomarkers for both Aβ and neuronal injury suggest that the MCI syndrome is not due to AD. In such instances the subjects may not be well suited for the methods described herein.

There is evidence that magnetic resonance imaging can observe deterioration, including progressive loss of gray matter in the brain, from mild cognitive impairment to full-blown Alzheimer disease (see, e.g., Whitwell et al. (2008) Neurology 70(7): 512-520). A technique known as PiB PET imaging is used to clearly show the sites and shapes of beta amyloid deposits in living subjects using a C11 tracer that binds selectively to such deposits (see, e.g., Jack et al. (2008) Brain 131(Pt 3): 665-680).

In certain embodiments, MCI is typically diagnosed when there is 1) Evidence of memory impairment; 2) Preservation of general cognitive and functional abilities; and 3) Absence of diagnosed dementia.

In certain embodiments MCI and stages of Alzheimer's disease can be identified/categorized, in part by Clinical Dementia Rating (CDR) scores. The CDR is a five point scale used to characterize six domains of cognitive and functional performance applicable to Alzheimer disease and related dementias: Memory, Orientation, Judgment & Problem Solving, Community Affairs, Home & Hobbies, and Personal Care. The information to make each rating can be obtained through a semi-structured interview of the patient and a reliable informant or collateral source (e.g., family member).

The CDR table provides descriptive anchors that guide the clinician in making appropriate ratings based on interview data and clinical judgment. In addition to ratings for each domain, an overall CDR score may be calculated through the use of an algorithm. This score is useful for characterizing and tracking a patient's level of impairment/dementia: 0=Normal; 0.5=Very Mild Dementia; 1=Mild Dementia; 2=Moderate Dementia; and 3=Severe Dementia. An illustrative CDR table is shown in Table 1.

TABLE 1
Illustrative clinical dementia rating (CDR) table.

| | Impairment: | | | | |
|---|---|---|---|---|---|
| | None | Questionable | Mild | Moderate | Severe |
| | CDR: | | | | |
| | 0 | 0.5 | 1 | 2 | 3 |
| Memory | No memory | Consistent | Moderate | Severe | Severe |
| | loss or slight | slight | memory loss; | memory | memory |
| | inconsistent | forgetfulness; | more marked | loss; only | loss; only |
| | forgetfulness | partial | for recent | highly | fragments |
| | | recollection | events; defect | learned | remain |
| | | of events' | interferes | material | |
| | | “benign” | with | retained; | |
| | | forgetfulness | everyday | new material | |
| | | | activities | rapidly lost | |
| Orientation | Fully | Fully | Moderate | Severe | Oriented to |
| | oriented | oriented | difficulty | difficulty | person only |
| | | except for | with time | with time | |
| | | slight | relationships; | relationships; | |
| | | difficulty | oriented for | usually | |
| | | with time | place at | disoriented | |
| | | relationships | examination; | to time, often | |
| | | | may have | to place. | |
| | | | geographic | | |
| | | | disorientation | | |
| | | | elsewhere | | |
| Judgment & | Solves | Slight | Moderate | Severely | Unable to |
| Problem | everyday | impairment | difficulty in | impaired in | make |
| Solving | problems & | in solving | handling | handling | judgments |
| | handles | problems, | problems, | problems, | or solve |
| | business & | similarities, | similarities | similarities | problems |
| | financial | and | and | and | |
| | affairs well; | differences | differences; | differences; | |
| | judgment | | social | social | |
| | good in | | judgment | judgment | |
| | relation to | | usually | usually | |
| | past | | maintained | impaired | |
| | performance | | | | |
| Community | Independent | Slight | Unable to | No pretense of independent | |
| Affairs | function at | impairment | function | function outside of home | |
| | usual level | in these | independently | Appears well | Appears too |
| | in job, | activities | at these | enough to be | ill to be |
| | shopping, | | activities | taken to | taken to |
| | volunteer, | | although may | functions | functions |
| | and social | | still be | outside a | outside a |
| | groups | | engaged in | family home | family |
| | | | some; | | home. |
| | | | appears | | |
| | | | normal to | | |
| | | | casual | | |
| | | | inspection | | |
| Home and | Life at | Life at home, | Mild bit | Only simple | No |
| Hobbies | home, | hobbies, and | definite | chores | significant |
| | hobbies, and | intellectual | impairment | preserved; | function in |
| | intellectual | interests | of function at | very | home |
| | interests | slightly | home; more | restricted | |
| | well | impaired | difficult | interests, | |
| | maintained | | chores | poorly | |
| | | | abandoned; | maintained | |
| | | | more | | |
| | | | complicated | | |
| | | | hobbies and | | |
| | | | interests | | |
| | | | abandoned | | |

| | | | | |
|---|---|---|---|---|
| Personal | Fully capable of self-care | Needs | Requires | Requires |
| Care | | prompting | assistance in | much help |
| | | | dressing, | with |
| | | | hygiene, | personal |
| | | | keeping of | care; |
| | | | personal | frequent |
| | | | effects | incontinence |

A CDR rating of ˜0.5 or ˜0.5 to 1.0 is often considered clinically relevant MCI. Higher CDR ratings can be indicative of progression into Alzheimer's disease.

In certain embodiments administration of one or more agents described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) is deemed effective when there is a measurable increase in SirT1, and/or an increase in ADAM10, and/or a normalization in SirT1/SirT2 ratios, and/or a reduction in the CSF of levels of one or more components selected from the group consisting of Tau, phospho-Tau (pTau), APPneo, soluble Aβ40, soluble Aβ42, and/or Aβ42/Aβ40 ratio, and/or when there is a reduction of the plaque load in the brain of the subject, and/or when there is a reduction in the rate of plaque formation in the brain of the subject, and/or when there is an improvement in the cognitive abilities of the subject, and/or when there is a perceived improvement in quality of life by the subject, and/or when there is a significant reduction in clinical dementia rating (CDR), and/or when the rate of increase in clinical dementia rating is slowed or stopped and/or when the progression from MCI to early stage AD is slowed or stopped.

In some embodiments, a diagnosis of MCI can be determined by considering the results of several clinical tests. For example, Grundman, et al. (2004) Arch Neurol 61: 59-66, report that a diagnosis of MCI can be established with clinical efficiency using a simple memory test (paragraph recall) to establish an objective memory deficit, a measure of general cognition (Mini-Mental State Exam (MMSE), discussed in greater detail below) to exclude a broader cognitive decline beyond memory, and a structured clinical interview (CDR) with patients and caregivers to verify the patient's memory complaint and memory loss and to ensure that the patient was not demented. Patients with MCI perform, on average, less than 1 standard deviation (SD) below normal on nonmemorycognitive measures included in the battery. Tests of learning, attention, perceptual speed, category fluency, and executive function may be impaired in patients with MCI, but these are far less prominent than the memory deficit.

Alzheimer's Disease (AD).

In certain embodiments the active agent(s (e.g., alaproclate and other compounds described herein, or a tautomer(s) or stereoisomer(s) thereof, or pharmaceutically acceptable salts or solvates of said alaproclate and alaproclate analogs, said stereoisomer(s), or said tautomer(s), or analogues, derivatives, or prodrugs thereof) are contemplated for the treatment of Alzheimer's disease. In such instances the methods described herein are useful in preventing or slowing the onset of Alzheimer's disease (AD), in reducing the severity of AD when the subject has transitioned to clinical AD diagnosis, and/or in mitigating one or more symptoms of Alzheimer's disease.

In particular, where the Alzheimer's disease is early stage, the methods can reduce or eliminate one or more symptoms characteristic of AD and/or delay or prevent the progression from MCI to early or later stage Alzheimer's disease.

Individuals presently suffering from Alzheimer's disease can be recognized from characteristic dementia, as well as the presence of risk factors described above. In addition, a number of diagnostic tests are available for identifying individuals who have AD. Individuals presently suffering from Alzheimer's disease can be recognized from characteristic dementia, as well as the presence of risk factors described above. In addition, a number of diagnostic tests are available for identifying individuals who have AD. These include measurement of CSF Tau, phospho-tau (pTau), sAPPα, sAPPβ, Aβ40, Aβ42 levels and/or C terminally cleaved APP fragment (APPneo). Elevated Tau, pTau, sAPPβ and/or APPneo, and/or decreased sAPPα, soluble Aβ40 and/or soluble Aβ42 levels, particularly in the context of a differential diagnosis, can signify the presence of AD.

In certain embodiments subjects amenable to treatment may have Alzheimer's disease. Individuals suffering from Alzheimer's disease can also be diagnosed by Alzheimer's disease and Related Disorders Association (ADRDA) criteria. The NINCDS-ADRDA Alzheimer's Criteria were proposed in 1984 by the National Institute of Neurological and Communicative Disorders and Stroke and the Alzheimer's Disease and Related Disorders Association (now known as the Alzheimer's Association) and are among the most used in the diagnosis of Alzheimer's disease (AD). McKhann, et al. (1984) Neurology 34(7): 939-44. According to these criteria, the presence of cognitive impairment and a suspected dementia syndrome should be confirmed by neuropsychological testing for a clinical diagnosis of possible or probable AD. However, histopathologic confirmation (microscopic examination of brain tissue) is generally used for a dispositive diagnosis. The NINCDS-ADRDA Alzheimer's Criteria specify eight cognitive domains that may be impaired in AD: memory, language, perceptual skills, attention, constructive abilities, orientation, problem solving and functional abilities). These criteria have shown good reliability and validity.

Baseline evaluations of patient function can made using classic psychometric measures, such as the Mini-Mental State Exam (MMSE) (Folstein et al. (1975) J. Psychiatric Research 12 (3): 189-198), and the Alzheimer's Disease Assessment Scale (ADAS), which is a comprehensive scale for evaluating patients with Alzheimer's Disease status and function (see, e.g., Rosen, et al. (1984) Am. J. Psychiatr., 141: 1356-1364).

These psychometric scales provide a measure of progression of the Alzheimer's condition. Suitable qualitative life scales can also be used to monitor treatment. The extent of disease progression can be determined using a Mini-Mental State Exam (MMSE) (see, e.g., Folstein, et al. supra). Any score greater than or equal to 25 points (out of 30) is effectively normal (intact). Below this, scores can indicate severe (≦9 points), moderate (10-20 points) or mild (21-24 points) Alzheimer's disease.

Alzheimer's disease can be broken down into various stages including: 1) Moderate cognitive decline (Mild or early-stage Alzheimer's disease), 2) Moderately severe cognitive decline (Moderate or mid-stage Alzheimer's disease), 3) Severe cognitive decline (Moderately severe or mid-stage Alzheimer's disease), and 4) Very severe cognitive decline (Severe or late-stage Alzheimer's disease) as shown in Table 2.

TABLE 2
Illustrative stages of Alzheimer's disease.

Moderate Cognitive Decline (Mild or early stage AD)
At this stage, a careful medical interview detects clear-cut deficiencies in
the following areas:
Decreased knowledge of recent events.
Impaired ability to perform challenging mental arithmetic. For
example, to count backward from 100 by 7s.
Decreased capacity to perform complex tasks, such as marketing,
planning dinner for guests, or paying bills and managing finances.
Reduced memory of personal history.
The affected individual may seem subdued and withdrawn,
especially in socially or mentally challenging situations.
Moderately severe cognitive decline (Moderate or mid-stage Alzheimer's
disease)
Major gaps in memory and deficits in cognitive function emerge. Some
assistance with day-to-day activities becomes essential. At this stage,
individuals may:
Be unable during a medical interview to recall such important
details as their current address, their telephone number, or the name of
the college or high school from which they graduated.
Become confused about where they are or about the date, day of the
week or season.
Have trouble with less challenging mental arithmetic; for example,
counting backward from 40 by 4s or from 20 by 2s.
Need help choosing proper clothing for the season or the occasion.
Usually retain substantial knowledge about themselves and know
their own name and the names of their spouse or children.
Usually require no assistance with eating or using the toilet.
Severe cognitive decline (Moderately severe
or mid-stage Alzheimer's disease)
Memory difficulties continue to worsen, significant personality changes
may emerge, and affected individuals need extensive help with daily
activities. At this stage, individuals may:
Lose most awareness of recent experiences and events as well as of
their surroundings.
Recollect their personal history imperfectly, although they generally
recall their own name.
Occasionally forget the name of their spouse or primary caregiver
but generally can distinguish familiar from unfamiliar faces.
Need help getting dressed properly; without supervision, may make
such errors as putting pajamas over daytime clothes or shoes on wrong
feet.
Experience disruption of their normal sleep/waking cycle.
Need help with handling details of toileting (flushing toilet, wiping
and disposing of tissue properly).
Have increasing episodes of urinary or fecal incontinence.
Experience significant personality changes and behavioral
symptoms, including suspiciousness and delusions (for example, believing
that their caregiver is an impostor); hallucinations (seeing or hearing
things that are not really there); or compulsive, repetitive behaviors such
as hand-wringing or tissue shredding.
Tend to wander and become lost.
Very severe cognitive decline (Severe or late-stage Alzheimer's disease)
This is the final stage of the disease when individuals lose the ability to
respond to their environment, the ability to speak, and, ultimately, the
ability to control movement.
Frequently individuals lose their capacity for recognizable speech,
although words or phrases may occasionally be uttered.
Individuals need help with eating and toileting and there is general
incontinence.
Individuals lose the ability to walk without assistance, then the
ability to sit without support, the ability to smile, and the ability to hold
their head up. Reflexes become abnormal and muscles grow rigid.
Swallowing is impaired.

In various embodiments administration of one or more agents described herein to subjects diagnosed with Alzheimer's disease is deemed effective when the there is a reduction in the CSF of levels of one or more components selected from the group consisting of Tau, phospho-Tau (pTau), APPneo, soluble Aβ40, soluble Aβ42, and/or and Aβ42/Aβ40 ratio, and/or when there is a reduction of the plaque load in the brain of the subject, and/or when there is a reduction in the rate of plaque formation in the brain of the subject, and/or when there is an improvement in the cognitive abilities of the subject, and/or when there is a perceived improvement in quality of life by the subject, and/or when there is a significant reduction in clinical dementia rating (CDR) of the subject, and/or when the rate of increase in clinical dementia rating is slowed or stopped and/or when the progression of AD is slowed or stopped (e.g., when the transition from one stage to another as listed in Table 3 is slowed or stopped).

In certain embodiments subjects amenable to the present methods generally are free of a neurological disease or disorder other than Alzheimer's disease. For example, in certain embodiments, the subject does not have and is not at risk of developing a neurological disease or disorder such as Parkinson's disease, and/or schizophrenia, and/or psychosis.

### Active Agent(s).

The methods described herein are based, in part, on the discovery that administration of one or more active agents (e.g., alaproclate and other compounds described herein, or a tautomer(s) or stereoisomer(s) thereof, or pharmaceutically acceptable salts or solvates of said alaproclate and other compounds described herein, said stereoisomer(s), or said tautomer(s), or analogues, derivatives, or prodrugs thereof) find use in the treatment and/or prophylaxis of diseases characterized by decreased sirtuins levels (e.g., SirT1), and/or decreased ADAM10 levels, and/or amyloid deposits in the brain, for example, mild cognitive impairment, Alzheimer's disease, macular degeneration, and the like.

In various embodiments, the active agents contemplated herein include, but are not limited to analogs of alaproclate. In various embodiments, suitable analogs reverse (a) ApoE4-mediated APP-Thr phosphorylation and tauphosphorylation and (b) ApoE4-mediated reduction in SirT1 expression (mRNA and protein) and activity. In various embodiments the analogs are formed by as by adding polar groups, and increasing permeability by adding suitable lipophilic groups. Typically, the compounds comply with “Lipinski's Rule of Five”. In certain embodiments, the compounds are substantially pure S-(−) enantiomers or substantially pure R-(+)-enantiomers.

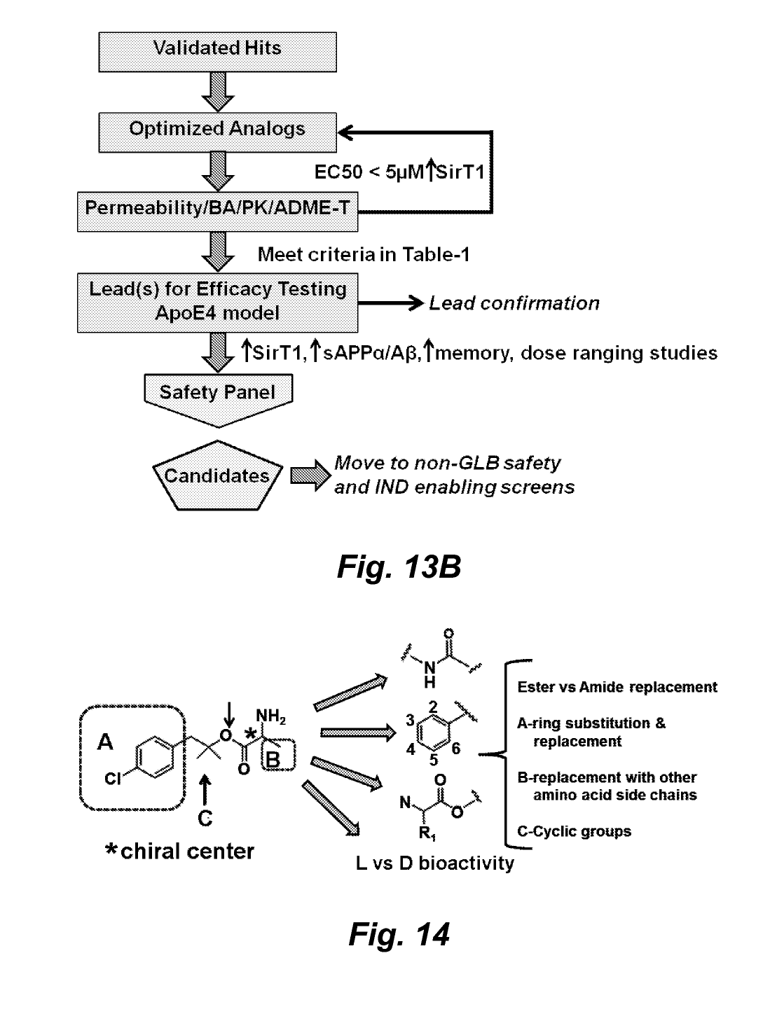

Illustrative, but non-limiting, modifications of alaproclate are shown in FIG. 14 and illustrative, but non-limiting substitutions for the A-region, B-region, and amide analogs are shown in Table 3.

In certain embodiments the active agents contemplated herein comprise a compound according to the formula:

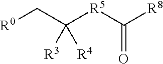

or a pharmaceutically acceptable salt thereof, where R⁸ is selected from the group consisting of

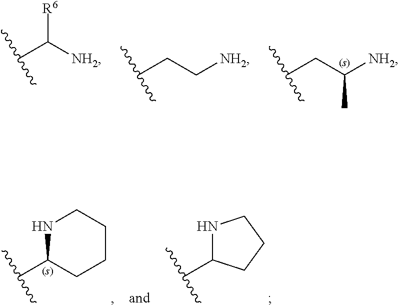

R⁰ is a substituted or unsubstituted cyclic or heterocycle selected from the group consisting of pyridine, pyrimidine, naphthalene, quinolone, isoquinoline, cinnoline, phenyl, substituted phenyl, oxazole, furan, isoxazole, thiazole, thiophene, pyrole, pyrazole, and imidazole; R³ and R⁴ are independently selected from the group consisting of hydrogen, methyl, ethyl, propyl, and butyl, or R³ taken with R⁴ and the carbon joining R³ and R⁴ form cyclohexane or cyclobutane; R⁵ is selected from the group consisting of O, NH, and NHR⁷, where R⁷ is a C1-C5 alkyl, or a cycloalkyl; R⁶ is selected from the group consisting the R-group (side chain) of one of the 20 natural amino acids, phenylglycine, and norleucine (see, e.g., Table 4); and R⁶ is not CH₃, or R³ and R⁴ are not both CH₃, or when R⁶ is CH₃, said compound is not a compound selected from the group consisting of

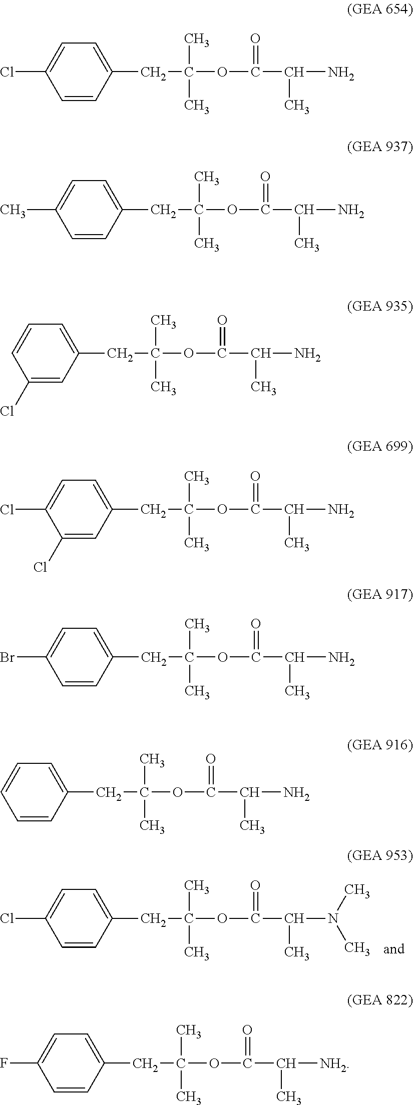

In certain embodiments the compound is not any of compounds 1, 2, 4, 5, 6, 7, 8, 11, 15 in Table 6. In certain embodiments the compound is not any one or more of compounds 1 through 24 in Table 6. In certain embodiments the compound is not any of the compounds in Table 6. In certain embodiments these active agents comprise a compound according to the formula:

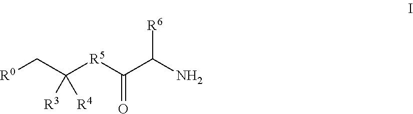

or a pharmaceutically acceptable salt thereof. In certain embodiments, R³ and R⁴ are independently selected from the group consisting of hydrogen, methyl, ethyl, propyl, and butyl.

In certain embodiments the compound has the formula

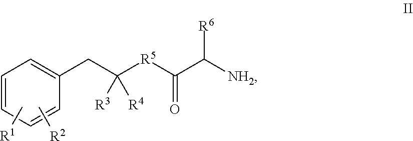

or a pharmaceutically acceptable salt thereof, where R¹ and R² are independently selected from the group consisting of hydrogen, halogen, alkyl having 1, 2 or 3 carbon atoms, and alkoxy having 1, 2 or 3 carbon atoms. In certain embodiments the compound has the formula

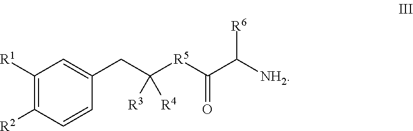

In certain embodiments in the compounds above R³ is CH₃. In certain embodiments, R³ is CH₃ and R⁴ is H. In certain embodiments, R³ and R⁴ are both H, and in certain embodiments, R³ and R⁴ are both CH₃. In certain embodiments, in any of the foregoing compounds R⁵ is O. In certain embodiments, in any of the foregoing compounds R⁵ is NH.

In certain embodiments in any of the foregoing compounds R¹ and R² are independently selected from the group consisting of hydrogen, halogen, and CH₃. In certain embodiments R¹ and R² are independently selected from the group consisting of H, Cl, and F. In certain embodiments R¹ is halogen and R² is H or R¹ is H and R² is halogen. In certain embodiments R¹ is Cl or F and R² is H. In certain embodiments R¹ and R² are both Cl or R¹ and R² are both F. In certain embodiments R¹ is Cl and R² is F, or R¹ is F and R² is Cl. In certain embodiments R¹ is H and R² is F or R¹ is F and R² is Cl. In certain embodiments R¹ is H and R² is Cl, or R¹ is Cl and R² is H. In certain embodiments R¹ is H and R² is CH₃, or R¹ is CH₃ and R² is H, in certain embodiments R¹ is F and R² is F, in certain embodiments R¹ is H and R² is F, in certain embodiments R¹ is F and R² is H.

In certain embodiments in any of the foregoing compounds, R⁶ is an amino acid R group selected from a naturally occurring amino acid shown in Table 5. In certain embodiments in any of the foregoing compounds R⁶ is selected from the group consisting of H, CH₃, —CH(CH₃)₂, —CH₂—CH(CH₃)₂, —CH₂-phenyl, CH2-substituted phenyl, —CH(CH₃)—CH₂CH₃, -phenyl, substituted phenyl, and —CH₂—CH₂—CH₂—CH₃.

TABLE 5
Illustrative groups for R⁶.

| Amino Acid | R-Group |
|---|---|
| alanine | —CH₃ |
| valine | 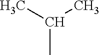 |
| leucine | 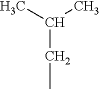 |
| isoleuciine | 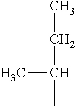 |
| proline* | 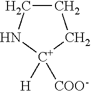 |
| phenylalanine | 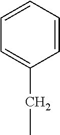 |
| tryptophpan | 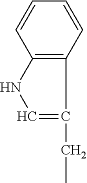 |
| methionine | —CH₂—CH₂—S—CH₃ |
| glycine | —H |
| serine | —CH₂—OH |
| threonine | 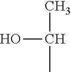 |
| cysteine | —CH₂—SH |
| tyrosine | 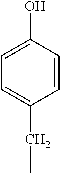 |
| asparagine | 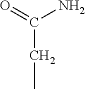 |
| glutamine | 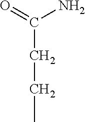 |
| aspartic acid | 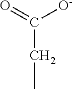 |
| glutamic acid | 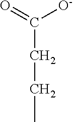 |
| lysine | —CH₂—CH₂—CH₂—CH₂—NH₃⁺ |
| arginine | 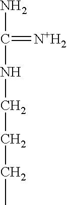 |
| histidine | 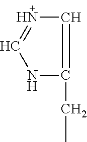 |

*shown with alpha carbon.

In certain embodiments where the carboxylic acid of aspartic acid and glutamic acid is replaced by a carboxylate ester.

In certain embodiments the compound comprise any one of compounds 1-24 shown in Table 6.

The foregoing compounds are illustrative and non-limiting. Using the teachings provided herein, other alaproclate-related active agents will be available to one of skill in the art.

It is also noted that in certain embodiments, the various prophylactic and/or therapeutic methods described herein contemplate use of one or more of the compounds described above and/or one or more compounds selected from the group consisting of GEA 654, GEA 937, GEA 935, GEA 699, GEA 917, GEA 916, GEA 953, and GEA 822 as described in U.S. Pat. No. 4,237,311 and as identified herein, alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), and 2-(4-chlorophenyl)-1,1-dimethylethyl 2-amino-3-methylbutanoate (GEA 857).

Compound Synthesis.

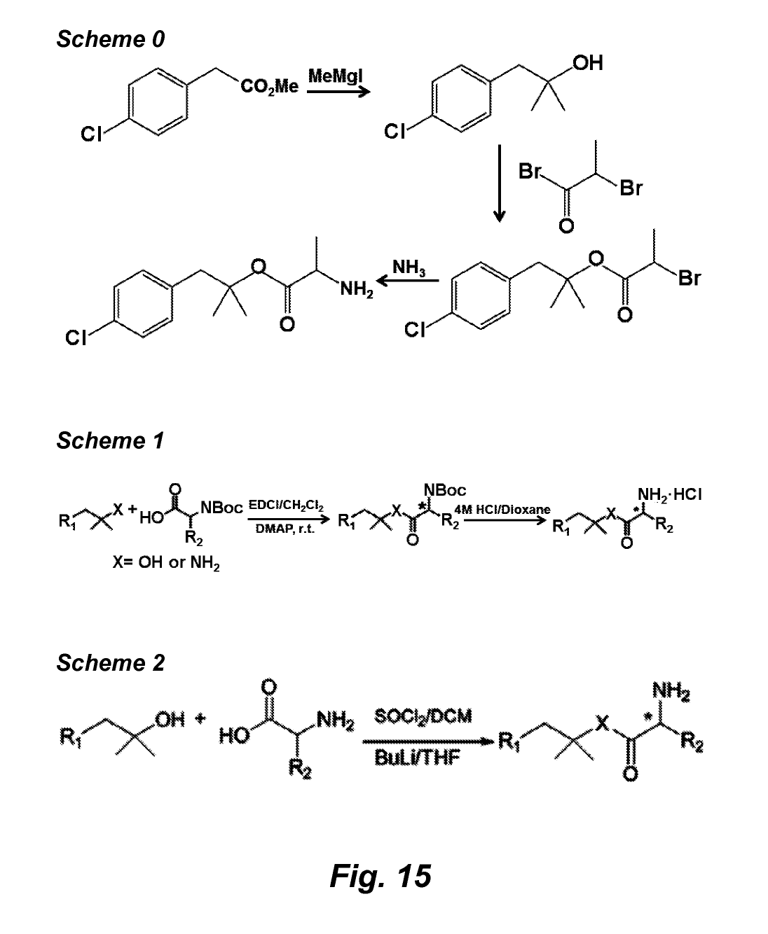

The compounds described herein can readily be synthesized using methods known to those of skill in the art. For example, scheme 0 shown in FIG. 15 is a well known synthesis scheme for alaproclate described, for example, by Lindberg et al. (1978) J. Med. Chem., 21(5): 448-456. Additionally, U.S. Pat. No. 4,469,707 and U.S. Pat. No. 4,237,311, incorporated herein by references for the compounds and synthesis methods described therein, teach the synthesis of alaproclate and numerous analogs.

As indicated above, with respect to the strategies illustrated in FIG. 14 a number of compounds contemplated herein comprises variations in regions A and B as esters or amides, e.g., as shown above in Table 3.

The synthesis of these analogs occurs from commercially available starting materials and can take place, for example, according to Scheme 1, shown in FIG. 15 which is quite amenable to the ester synthesis. The ring (e.g., the phenyl ring (A) shown in FIG. 14 can be functionalized by selected electron releasing/withdrawing substitutions, bioisosteric replacements including heterocyclic analogs, and replacement of the side chain of the amino acids. Manipulating the solubility, lipophilicity and protein binding of the molecules can be done in an iterative fashion to affect all the properties associated with absorption, distribution metabolism, and excretion (ADME) and increase bioavailability and brain uptake.

One illustrative, but non-limiting procedure involves use of the Boc-amino acid in CH₂Cl₂ in the presence of 1 equivalent of the alcohol and 1.5 equivalent of EDCI in portions along with catalytic amounts of DMAP (5 mol %). Stirring for 12 hrs at room temperature and standard workup would typically yields the protected ester. The deprotection of the amino group with dioxane and HCl and azeotroping removes all dioxane yielding the amino acid ester.

The same procedure can be used for the amide analogs, except the deprotection can be done with TFA/CH₂Cl₂ and has been reported to produce high yields of the resultant esters.

In another procedure (described by Hanzawa et al. (2012) J. Oleo Sci. 61(11): 631-640) in the first step of synthesis, the amino acyl hydrochlorides (AAHCls) are prepared using amino acid in DCM and thionyl chloride, followed by the addition of the anion of the alcohol at 0° C. in THF slowly added to the mixture. In the next step, the reaction is worked with water and bicarbonate. After the completion of the workup, water is removed under vacuum, and ester is extracted by the addition of dichloromethane (see, e.g., Scheme 2 in FIG. 15). Another approach is to use Z-protected amino acid to yield acid chloride, reaction with the alcohol in THF and triethylamine, and deprotection by hydrogenation. Specific examples of preparation of JP-01-025 shown in Table 5 can be prepared using the synthesis shown in Scheme 1.

It will be recognized that these synthesis strategies are illustrative and non-limiting. Using the teaching provided herein numerous alternative synthesis approaches will be available to one of skill in the art.

### Assays to Validate Active Agents.

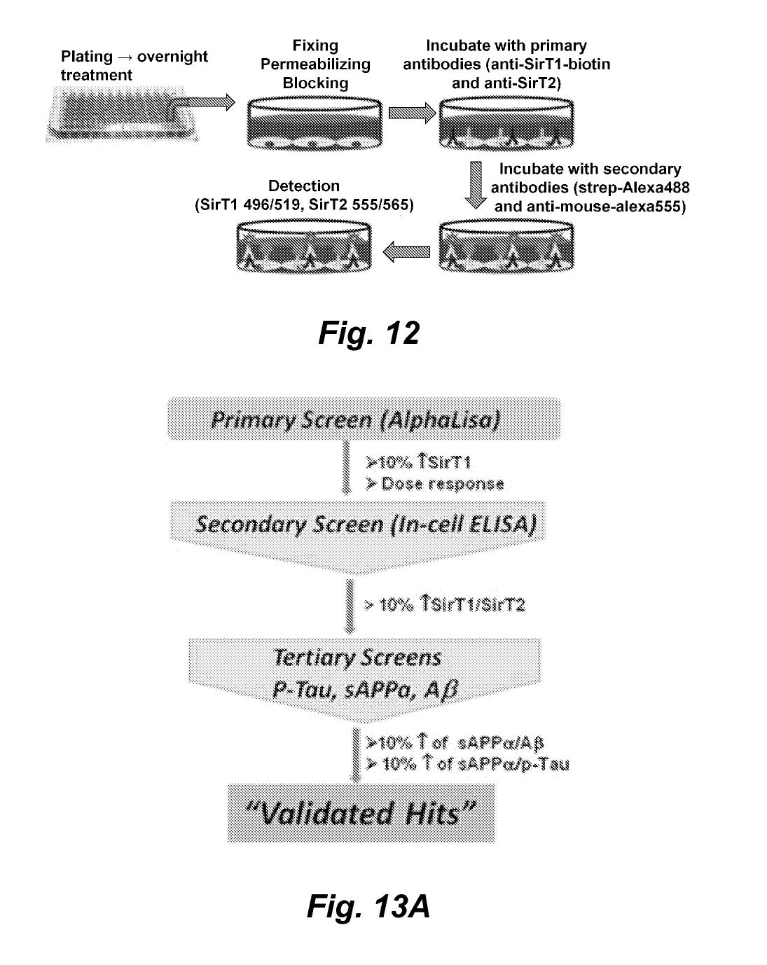

Assays to validate the active agents are illustrated in FIGS. 13A and 13B and described in detail in the Examples. As shown therein, compounds can be screened in a primary AlphaLISA assay to identify compounds that increase SirT1. An in-cell ELISA can be used to determine SirT1/SirT2 ratios in the secondary screen; and a neuronal cell line such as SH-SY5Y will be used in the tertiary assay for the biomarkers p-tau, sAPPα, sAPPβ, Aβ1-42, and therefore the sAPPα/Aβ and sAPPα/sAPPβ ratios.

Leads can be evaluated in permeability assays for brain uptake after oral delivery, in in vitro ADME/T assays and molecular mechanism studies. Permeability can be evaluated by parallel artificial membrane permeability assay (PAMPA), brain compound levels after oral dosing by pharmacokinetic (PK) analysis, other in vitro ADME properties can be determined, and studies performed to elucidate the mechanism(s) by which candidates enhance SirT1. An ApoE4-AD mouse model will be used to ascertain lead candidates' ability to increase SirT1 levels in plasma and brain, to improve in biomarker levels and ratios, and to improve learning and memory.

### Pharmaceutical Formulations.

In certain embodiments one or more active agents described herein (e.g., alaproclate and other “related” compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) are administered to a mammal in need thereof, e.g., to a mammal at risk for or suffering from a pathology characterized by reduced sirtuins (e.g., SirT1) expression, and/or reduced ADAM10 expression, and/or abnormal processing of amyloid precursor proteins, a mammal at risk for progression of MCI to Alzheimer's disease, and so forth. In certain embodiments the active agent(s) are administered to prevent or delay the onset of a pre-Alzheimer's condition and/or cognitive dysfunction, and/or to ameliorate one or more symptoms of a pre-Alzheimer's cognitive dysfunction, and/or to prevent or delay the progression of a pre-Alzheimer's condition or cognitive dysfunction to Alzheimer's disease, and/or to promote the processing of amyloid precursor protein (APP) by a non-amyloidogenic pathway.

In certain embodiments one or more active agents described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) are administered to a mammal in need thereof, e.g., to a mammal at risk for or suffering from a pathology characterized by reduced expression or activity of sirtuins (e.g., SirT1) and/or reduced ADAM10 expression or activity, and/or abnormal processing of amyloid precursor proteins or increased tau and p-tau in conditions other than Alzheimer's disease of MCI. Illustrative conditions, include, but are not limited to AD-type symptoms of patients with Down's syndrome, glaucoma, macular degeneration (e.g., age-related macular degeneration (AMD), olfactory impairment. in the treatment of type-II diabetes, including diabetes associated with amyloidogenesis, neurodegenerative diseases such as scrapie, bovine spongiform encaphalopathies (e.g., BSE), traumatic brain injury (“TBI”), Creutzfeld-Jakob disease and the like, type II diabetes, chronic traumatic encelphalopathy (CTE). Other conditions characterized by characterized by amyloid formation/deposition are contemplated. Such conditions include, but are not limited to Huntington's Disease, medullary carcinoma of the thyroid, cardiac arrhythmias, isolated atrial amyloidosis, atherosclerosis, rheumatoid arthritis, aortic medial amyloid, prolactinomas, familial amyloid polyneuropathy, hereditary non-neuropathic systemic amyloidosis, dialysis related amyloidosis, Finnish amyloidosis, Lattice corneal dystrophy, cerebral amyloid angiopathy (e.g., Icelandic type), systemic AL amyloidosis, sporadic inclusion body myositis, cerebrovascular dementia, and the like.

The active agent(s) (e.g., alaproclate and other compounds described herein) can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience, and as described above.

For example, a pharmaceutically acceptable salt can be prepared for any of the agent(s) described herein having a functionality capable of forming a salt. A pharmaceutically acceptable salt is any salt that retains the activity of the parent compound and does not impart any deleterious or untoward effect on the subject to which it is administered and in the context in which it is administered.

In various embodiments pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules.

Methods of formulating pharmaceutically active agents as salts, esters, amide, prodrugs, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) are useful for parenteral administration, topical administration, oral administration, nasal administration (or otherwise inhaled), rectal administration, or local administration, such as by aerosol or transdermally, for prophylactic and/or therapeutic treatment of one or more of the pathologies/indications described herein (e.g., pathologies characterized by excess amyloid plaque formation and/or deposition or undesired amyloid or pre-amyloid processing).

In various embodiments the active agents described herein can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disintegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) and the resulting composition is compressed. Where necessary the compressed product is coated, e.g., using known methods for masking the taste or for enteric dissolution or sustained release. Suitable coating materials include, but are not limited to ethyl-cellulose, hydroxymethylcellulose, POLYOX®yethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physiochemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, mucoadherent films, topical varnishes, lipid complexes, etc.

Pharmaceutical compositions comprising the active agents described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

In certain embodiments, the active agents described herein are formulated for oral administration. For oral administration, suitable formulations can be readily formulated by combining the active agent(s) with pharmaceutically acceptable carriers suitable for oral delivery well known in the art. Such carriers enable the active agent(s) described herein to be formulated as tablets, pills, dragees, caplets, lizenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

For administration by inhalation, the active agent(s) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In various embodiments the active agent(s) can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45, amphiphilic materials such as Suppocire AP and polyglycolized glyceride, and the like). The base is selected and compounded for a desired melting/delivery profile.

For topical administration the active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) can be formulated as solutions, gels, ointments, creams, suspensions, and the like as are well-known in the art.

In certain embodiments the active agents described herein are formulated for systemic administration (e.g., as an injectable) in accordance with standard methods well known to those of skill in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. Such penetrants are generally known in the art. Injectable formulations and inhalable formulations are generally provided as a sterile or substantially sterile formulation.

In addition to the formulations described previously, the active agent(s) may also be formulated as a depot preparations. Such long acting formulations can be administered by implantation (for example subcutaneously or intramuscularly) or by intramuscular injection. Thus, for example, the active agent(s) may be formulated with suitable polymeric or hydrophobic materials (for example as an emulsion in an acceptable oil) or ion exchange resins, or as sparingly soluble derivatives, for example, as a sparingly soluble salt.

In certain embodiments the active agent(s) described herein can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Alternatively, other pharmaceutical delivery systems can be employed. For example, liposomes, emulsions, and microemulsions/nanoemulsions are well known examples of delivery vehicles that may be used to protect and deliver pharmaceutically active compounds. Certain organic solvents such as dimethylsulfoxide also can be employed, although usually at the cost of greater toxicity.

In certain embodiments the active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) are formulated in a nanoemulsion. Nanoemulsions include, but are not limited to oil in water (0/W) nanoemulsions, and water in oil (W/0) nanoemulsions. Nanoemulsions can be defined as emulsions with mean droplet diameters ranging from about 20 to about 1000 nm. Usually, the average droplet size is between about 20 nm or 50 nm and about 500 nm. The terms sub-micron emulsion (SME) and mini-emulsion are used as synonyms.

Illustrative oil in water (O/W) nanoemulsions include, but are not limited to: Surfactant micelles—micelles composed of small molecules surfactants or detergents (e.g., SDS/PBS/2-propanol); Polymer micelles—micelles composed of polymer, copolymer, or block copolymer surfactants (e.g., Pluronic L64/PBS/2-propanol); Blended micelles—micelles in which there is more than one surfactant component or in which one of the liquid phases (generally an alcohol or fatty acid compound) participates in the formation of the micelle (e.g., octanoic acid/PBS/EtOH); Integral micelles—blended micelles in which the active agent(s) serve as an auxiliary surfactant, forming an integral part of the micelle; and Pickering (solid phase) emulsions—emulsions in which the active agent(s) are associated with the exterior of a solid nanoparticle (e.g., polystyrene nanoparticles/PBS/no oil phase).

Illustrative water in oil (W/O) nanoemulsions include, but are not limited to: Surfactant micelles—micelles composed of small molecules surfactants or detergents (e.g., dioctyl sulfosuccinate/PBS/2-propanol, isopropylmyristate/PBS/2-propanol, etc.); Polymer micelles—micelles composed of polymer, copolymer, or block copolymer surfactants (e.g., PLURONIC® L121/PBS/2-propanol); Blended micelles—micelles in which there is more than one surfactant component or in which one of the liquid phases (generally an alcohol or fatty acid compound) participates in the formation of the micelle (e.g., capric/caprylic diglyceride/PBS/EtOH); Integral micelles—blended micelles in which the active agent(s) serve as an auxiliary surfactant, forming an integral part of the micelle (e.g., active agent/PBS/polypropylene glycol); and Pickering (solid phase) emulsions—emulsions in which the active agent(s) are associated with the exterior of a solid nanoparticle (e.g., chitosan nanoparticles/no aqueous phase/mineral oil).

As indicated above, in certain embodiments the nanoemulsions comprise one or more surfactants or detergents. In some embodiments the surfactant is a non-anionic detergent (e.g., a polysorbate surfactant, a polyoxyethylene ether, etc.). Surfactants that find use in the present invention include, but are not limited to surfactants such as the TWEEN®, TRITON®, and TYLOXAPOL® families of compounds.

In certain embodiments the emulsions further comprise one or more cationic halogen containing compounds, including but not limited to, cetylpyridinium chloride. In still further embodiments, the compositions further comprise one or more compounds that increase the interaction (“interaction enhancers”) of the composition with microorganisms (e.g., chelating agents like ethylenediaminetetraacetic acid, or ethylenebis(oxyethylenenitrilo)tetraacetic acid in a buffer).

In some embodiments, the nanoemulsion further comprises an emulsifying agent to aid in the formation of the emulsion. Emulsifying agents include compounds that aggregate at the oil/water interface to form a kind of continuous membrane that prevents direct contact between two adjacent droplets. Certain embodiments of the present invention feature oil-in-water emulsion compositions that may readily be diluted with water to a desired concentration without impairing their anti-pathogenic properties.

In addition to discrete oil droplets dispersed in an aqueous phase, certain oil-in-water emulsions can also contain other lipid structures, such as small lipid vesicles (e.g., lipid spheres that often consist of several substantially concentric lipid bilayers separated from each other by layers of aqueous phase), micelles (e.g., amphiphilic molecules in small clusters of 50-200 molecules arranged so that the polar head groups face outward toward the aqueous phase and the apolar tails are sequestered inward away from the aqueous phase), or lamellar phases (lipid dispersions in which each particle consists of parallel amphiphilic bilayers separated by thin films of water).

These lipid structures are formed as a result of hydrophobic forces that drive apolar residues (e.g., long hydrocarbon chains) away from water. The above lipid preparations can generally be described as surfactant lipid preparations (SLPs). SLPs are minimally toxic to mucous membranes and are believed to be metabolized within the small intestine (see e.g., Hamouda et al., (1998) J. Infect. Disease 180: 1939).

In certain embodiments the emulsion comprises a discontinuous oil phase distributed in an aqueous phase, a first component comprising an alcohol and/or glycerol, and a second component comprising a surfactant or a halogen-containing compound. The aqueous phase can comprise any type of aqueous phase including, but not limited to, water (e.g., dionized water, distilled water, tap water) and solutions (e.g., phosphate buffered saline solution or other buffer systems). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In certain embodiments, the oil phase comprises 30-90 vol % of the oil-in-water emulsion (e.g., constitutes 30-90% of the total volume of the final emulsion), more preferably 50-80%. The formulations need not be limited to particular surfactants, however in certain embodiments, the surfactant is a polysorbate surfactant (e.g., TWEEN 20®, TWEEN 40®, TWEEN 60®, and TWEEN 80®), a pheoxypolyethoxyethanol (e.g., TRITON® X-100, X-301, X-165, X-102, and X-200, and TYLOXAPOL®), or sodium dodecyl sulfate, and the like.

In certain embodiments a halogen-containing component is present. the nature of the halogen-containing compound, in some embodiments the halogen-containing compound comprises a chloride salt (e.g., NaCl, KCl, etc.), a cetylpyridinium halide, a cetyltrimethylammonium halide, a cetyldimethylethylammonium halide, a cetyldimethylbenzylammonium halide, a cetyltributylphosphonium halide, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyldimethylethylammonium bromide, cetyltributylphosphonium bromide, dodecyltrimethylammonium bromide, tetradecyltrimethylammonium bromide, and the like

In certain embodiments the emulsion comprises a quaternary ammonium compound. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl dimethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride;

didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Nanoemulsion formulations and methods of making such are well known to those of skill in the art and described for example in U.S. Pat. Nos. 7,476,393, 7,468,402, 7,314,624, 6,998,426, 6,902,737, 6,689,371, 6,541,018, 6,464,990, 6,461,625, 6,419,946, 6,413,527, 6,375,960, 6,335,022, 6,274,150, 6,120,778, 6,039,936, 5,925,341, 5,753,241, 5,698,219, and 5,152,923 and in Fanun et al. (2009) Microemulsions: Properties and Applications (Surfactant Science), CRC Press, Boca Ratan Fla.

In certain embodiments, one or more active agents described herein can be provided as a “concentrate”, e.g., in a storage container (e.g., in a premeasured volume) ready for dilution, or in a soluble capsule ready for addition to a volume of water, alcohol, hydrogen peroxide, or other diluent.

Extended Release (Sustained Release) Formulations.

In certain embodiments “extended release” formulations of the active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) are contemplated. In various embodiments such extended release formulations are designed to avoid the high peak plasma levels of intravenous and conventional immediate release oral dosage forms.

Illustrative sustained-release formulations include, for example, semipermeable matrices of solid polymers containing the therapeutic agent. Various uses of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization can be employed.

In certain embodiments such “extended release” formulations utilize the mucosa and can independently control tablet disintegration (or erosion) and/or drug dissolution and release from the tablet over time to provide a safer delivery profile. In certain embodiments the oral formulations of active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) provide individual, repetitive doses that include a defined amount of the active agent that is delivered over a defined amount of time.

One illustrative sustained release formulation is a substantially homogeneous composition that comprises about 0.01% to about 99% w/w, or about 0.1% to about 95%, or about 0.1%, or about 1%, or about 2%, or about 5%, or about 10%, or about 15%, or about 20% to about 80%, or to about 90%, or to about 95%, or to about 97%, or to about 98%, or to about 99%1 of the active ingredient(s) (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) and one or more mucoadhesives (also referred to herein as “bioadhesives”) that provide for adherence to the targeted mucosa of the subject (patient) and that may further comprise one or more of the following: one or more binders that provide binding of the excipients in a single tablet; one or more hydrogel forming excipients; one or more bulking agents; one or more lubricants; one or more glidants; one or more solubilizers; one or more surfactants; one or more flavors; one or more disintegrants; one or more buffering excipients; one or more coatings; one or more controlled release modifiers; and one or more other excipients and factors that modify and control the drug's dissolution or disintegration time and kinetics or protect the active drug from degradation.

In various embodiments a sustained release pharmaceutical dosage form for oral transmucosal delivery can be solid or non-solid. In one illustrative embodiment, the dosage form is a solid that turns into a hydrogel following contact with saliva.

Suitable excipients include, but are not limited to substances added to the formulations that are required to produce a commercial product and can include, but are not limited to: bulking agents, binders, surfactants, bioadhesives, lubricants, disintegrants, stabilizers, solubilizers, glidants, and additives or factors that affect dissolution or disintegration time. Suitable excipients are not limited to those above, and other suitable nontoxic pharmaceutically acceptable carriers for use in oral formulations can be found in Remington's Pharmaceutical Sciences, 17th Edition, 1985.

In certain embodiments extended release formulations of the active agent(s) described herein for oral transmucosal drug delivery include at least one bioadhesive (mucoadhesive) agent or a mixture of several bioadhesives to promote adhesion to the oral mucosa during drug delivery. In addition the bioadhesive agents may also be effective in controlling the dosage form erosion time and/or, the drug dissolution kinetics over time when the dosage form is wetted. Such mucoadhesive drug delivery systems are very beneficial, since they can prolong the residence time of the drug at the site of absorption and increase drug bioavailability. The mucoadhesive polymers forming hydrogels are typically hydrophilic and swellable, containing numerous hydrogen bond-forming groups, like hydroxyl, carboxyl or amine, which favor adhesion. When used in a dry form, they attract water from the mucosal surface and swell, leading to polymer/mucus interaction through hydrogen bonding, electrostatic, hydrophobic or van der Waals interaction.

Illustrative suitable mucoadhesive or bioadhesive materials, include, but are not limited to natural, synthetic or biological polymers, lipids, phospholipids, and the like. Examples of natural and/or synthetic polymers include cellulosic derivatives (such as methylcellulose, carboxymethyl cellulose, hydroxyethyl cellulose, hydroxyethylmethyl cellulose, etc), natural gums (such as guar gum, xanthan gum, locust bean gum, karaya gum, veegum etc.), polyacrylates (such as CARBOPOL®, polycarbophil, etc), alginates, thiol-containing polymers, POLYOX®yethylenes, polyethylene glycols (PEG) of all molecular weights (preferably between 1000 and 40,000 Da, of any chemistry, linear or branched), dextrans of all molecular weights (preferably between 1000 and 40,000 Da of any source), block copolymers, such as those prepared by combinations of lactic and glycolic acid (PLA, PGA, PLGA of various viscosities, molecular weights and lactic-to-glycolic acid ratios) polyethylene glycol-polypropylene glycol block copolymers of any number and combination of repeating units (such as PLURONICS®, TEKTRONIX® or GENAPOL® block copolymers), combination of the above copolymers either physically or chemically linked units (for example PEG-PLA or PEG-PLGA copolymers) mixtures. Preferably the bioadhesive excipient is selected from the group of polyethylene glycols, POLYOX®yethylenes, polyacrylic acid polymers, such as CARBOPOL® (such as CARBOPOL® 71G, 934P, 971P, 974P, and the like) and polycarbophils (such as NOVEON® AA-1, NOVEON® CA-1, NOVEON® CA-2, and the like), cellulose and its derivatives and most preferably it is polyethylene glycol, carbopol, and/or a cellulosic derivative or a combination thereof.

In certain embodiments the mucoadhesive/bioadhesive excipient is typically present at 1-50% w/w, preferably 1-40% w/w or most preferably between 5-30% w/w. A particular formulation may contain one or more different bioadhesives in any combination.

In certain embodiments the formulations for oral transmucosal drug delivery also include a binder or mixture of two or more binders which facilitate binding of the excipients into a single dosage form. Illustrative binders include, binders selected from the group consisting of cellulosic derivatives (such as methylcellulose, carboxymethyl cellulose, hydroxyethyl cellulose, hydroxyethylmethyl cellulose, etc.), polyacrylates (such as CARBOPOL®, polycarbophil, etc.), POVIDONE® (all grades), POLYOX®® of any molecular weight or grade, irradiated or not, starch, polyvinylpyrrolidone (PVP), AVICEL®, and the like. In certain embodiments the binder is typically present at 0.5-60% w/w, preferably 1-30% w/w and most preferably 1.5-15% w/w.

In certain embodiments the formulations also include at least one hydrogel-forming excipient. Illustrative hydrogel forming excipients include, but are not limited to those selected from the group consisting of polyethylene glycols and other polymers having an ethylene glycol backbone, whether homopolymers or cross linked heteropolymers, block copolymers using ethylene glycol units, such as POLYOX®yethylene homopolymers (such as POLYOX®® N10/MW=100,000 POLYOX®-80/MW=200,000; POLYOX® 1105/MW=900,000; POLYOX®-301/MW=4,000,000; POLYOX®-303/MW=7,000,000, POLYOX® WSR-N-60K, all of which are tradenames of Union Carbide), hydroxypropylmethylcellylose (HPMC) of all molecular weights and grades (such as METOLOSE® 90SH50000, METOLOSE® 90SH30000, all of which are tradenames of Shin-Etsu Chemical company), Poloxamers (such as LUTROL® F-68, LUTROL® F-127, F-105 etc., all tradenames of BASF Chemicals), GENAPOL®, polyethylene glycols (PEG, such as PEG-1500, PEG-3500, PEG-4000, PEG-6000, PEG-8000, PEG-12000, PEG-20,000, etc.), natural gums (xanthan gum, locust bean gum, etc.) and cellulose derivatives (HC, HMC, HMPC, HPC, CP, CMC), polyacrylic acid-based polymers either as free or cross-linked and combinations thereof, biodegradable polymers such as poly lactic acids, polyglycolic acids and any combination thereof, whether a physical blend or cross-linked. In certain embodiments, the hydrogel components may be cross-linked. The hydrogel forming excipient(s) are typically present at 0.1-70% w/w, preferably 1-50% w/w or most preferably 1-30% w/w.

In certain embodiments the formulations may also include at least one controlled release modifier which is a substance that upon hydration of the dosage form will preferentially adhere to the drug molecules and thus reduce the rate of its diffusion from the oral dosage form. Such excipients may also reduce the rate of water uptake by the formulation and thus enable a more prolonged drug dissolution and release from the tablet. In general the selected excipient(s) are lipophilic and capable of naturally complexing to the hydrophobic or lipophilic drugs. The degree of association of the release modifier and the drug can be varied by altering the modifier-to-drug ratio in the formulation. In addition, such interaction may be appropriately enhanced by the appropriate combination of the release modifier with the active drug in the manufacturing process. Alternatively, the controlled release modifier may be a charged polymer either synthetic or biopolymer bearing a net charge, either positive or negative, and which is capable of binding to the active via electrostatic interactions thus modifying both its diffusion through the tablet and/or the kinetics of its permeation through the mucosal surface. Similarly to the other compounds mentioned above, such interaction is reversible and does not involve permanent chemical bonds with the active. In certain embodiments the controlled release modifier may typically be present at 0-80% w/w, preferably 1-20% w/w, most preferably 1-10% w/w.

In various embodiments the extended release formulations may also include other conventional components required for the development of oral dosage forms, which are known to those skilled in the art. These components may include one or more bulking agents (such as lactose USP, Starch 1500, mannitol, sorbitol, malitol or other non-reducing sugars; microcrystalline cellulose (e.g., AVICEL®), dibasic calcium phosphate dehydrate, sucrose, and mixtures thereof), at least one solubilizing agent(s) (such as cyclodextrins, pH adjusters, salts and buffers, surfactants, fatty acids, phospholipids, metals of fatty acids etc.), metal salts and buffers organic (such as acetate, citrate, tartrate, etc.) or inorganic (phosphate, carbonate, bicarbonate, borate, sulfate, sulfite, bisulfite, metabisulfite, chloride, etc.), salts of metals such as sodium, potassium, calcium, magnesium, etc.), at least one lubricant (such as stearic acid and divalent cations of, such as magnesium stearate, calcium stearate, etc., talc, glycerol monostearate and the like), one or more glidants (such as colloidal silicon dioxide, precipitated silicon dioxide, fumed silica (CAB-O-SIL® M-5P, trademark of Cabot Corporation), stearowet and sterotex, silicas (such as SILOID® and SILOX® silicas—trademarks of Grace Davison Products, Aerosil—trademark of Degussa Pharma), higher fatty acids, the metal salts thereof, hydrogenated vegetable oils and the like), flavors or sweeteners and colorants (such as aspartame, mannitol, lactose, sucrose, other artificial sweeteners; ferric oxides and FD&C lakes), additives to help stabilize the drug substance from chemical of physical degradation (such as anti-oxidants, anti-hydrolytic agents, aggregation-blockers etc. Anti-oxidants may include BHT, BHA, vitamins, citric acid, EDTA, sodium bisulfate, sodium metabisulfate, thiourea, methionine, surfactants, amino-acids, such as arginine, glycine, histidine, methionine salts, pH adjusters, chelating agents and buffers in the dry or solution form), one or more excipients that may affect tablet disintegration kinetics and drug release from the tablet, and thus pharmacokinetics (disintegrants such as those known to those skilled in the art and may be selected from a group consisting of starch, carboxy-methycellulose type or crosslinked polyvinyl pyrrolidone (such as cross-povidone, PVP-XL), alginates, cellulose-based disintegrants (such as purified cellulose, methylcellulose, crosslinked sodium carboxy methylcellulose (Ac-Di-Sol) and carboxy methyl cellulose), low substituted hydroxypropyl ethers of cellulose, microcrystalline cellulose (such as AVICEL®), ion exchange resins (such as AMBRELITE® IPR 88), gums (such as agar, locust bean, karaya, pectin and tragacanth), guar gums, gum karaya, chitin and chitosan, smecta, gellan gum, isapghula husk, polacrillin potassium (Tulsion³³⁹), gas-evolving disintegrants (such as citric acid and tartaric acid along with the sodium bicarbonate, sodium carbonate, potassium bicarbonate or calcium carbonate), sodium starch glycolate (such as EXPLOTAB® and PRIMOGEL®), starch DC and the likes, at least one biodegradable polymer of any type useful for extended drug release. Exemplary polymer compositions include, but are not limited to, polyanhydrides and co-polymers of lactic acid and glycolic acid, poly(dl-lactide-co-glycolide) (PLGA), poly(lactic acid) (PLA), poly(glycolic acid) (PGA), polyorthoesters, proteins, and polysaccharides.

In certain embodiments, the active agent(s) can be chemically modified to significantly modify the pharmacokinetics in plasma. This may be accomplished for example by conjugation with poly(ethylene glycol) (PEG), including site-specific PEGylation. PEGylation, which may improve drug performance by optimizing pharmacokinetics, decreasing immunogenicity and dosing frequency.

Methods of making a formulation of the active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) for GI or oral transmucosal delivery are also provided. One method includes the steps of powder grinding, dry powder mixing and tableting via direct compression. Alternatively, a wet granulation process may be used. Such a method (such as high shear granulation process) involves mixing the active ingredient and possibly some excipients in a mixer. The binder may be one of the excipients added in the dry mix state or dissolved in the fluid used for granulating. The granulating solution or suspension is added to the dry powders in the mixer and mixed until the desired characteristics are achieved. This usually produces a granule that will be of suitable characteristics for producing dosage forms with adequate dissolution time, content uniformity, and other physical characteristics. After the wet granulation step, the product is most often dried and/or then milled after drying to get a major percentage of the product within a desired size range. Sometimes, the product is dried after being wet sized using a device such as an oscillating granulator, or a mill. The dry granulation may then processed to get an acceptable size range by first screening with a sieving device, and then milling the oversized particles.

Additionally, the formulation may be manufactured by alternative granulation processes, all known to those skilled in the art, such as spray fluid bed granulation, extrusion and spheronization or fluid bed rotor granulation.

Additionally, the tablet dosage form of the active agent(s) described herein may be prepared by coating the primary tablet manufactured as described above with suitable coatings known in the art. Such coatings are meant to protect the active cores against damage (abrasion, breakage, dust formation) against influences to which the cores are exposed during transport and storage (atmospheric humidity, temperature fluctuations), and naturally these film coatings can also be colored. The sealing effect of film coats against water vapor is expressed by the water vapor permeability. Coating may be performed by one of the available processes such as Wurster coating, dry coating, film coating, fluid bed coating, pan coating, etc. Typical coating materials include polyvinyl pyrrolidone (PVP), polyvinyl pyrrolidone vinyl acetate copolymer (PVPVA), polyvinyl alcohol (PVA), polyvinyl alcohol/polyethylene glycol copolymer (PVA/PEG), cellulose acetate phthalate, ethyl cellulose, gellan gum, maltodextrin, methacrylates, methyl cellulose, hydroxyl propyl methyl cellulose (HPMC of all grades and molecular weights), carrageenan, shellac and the like.

In certain embodiments the tablet core comprising the active agent(s) described herein can be coated with a bioadhesive and/or pH resistant material to enable material, such as those defined above, to improve bioadhesion of the tablet in the sublingual cavity.

In certain embodiments, the active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) are formulated as inclusion complexes. While not limited to cyclodextrin inclusion complexes, it is noted that cyclodextrin is the agent most frequently used to form pharmaceutical inclusion complexes. Cyclodextrins (CD) are cyclic oligomers of glucose, that typically contain 6, 7, or 8 glucose monomers joined by α-1,4 linkages. These oligomers are commonly called α-CD, β-CD, and γ-CD, respectively. Higher oligomers containing up to 12 glucose monomers are known, and contemplated to in the formulations described herein. Functionalized cyclodextrin inclusion complexes are also contemplated. Illustrative, but non-limiting functionalized cyclodextrins include, but are not limited to sulfonates, sulfonates and sulfinates, or disulfonates of hydroxybutenyl cyclodextrin; sulfonates, sulfonates and sulfinates, or disulfonates of mixed ethers of cyclodextrins where at least one of the ether substituents is hydroxybutenyl cyclodextrin. Illustrative cyclodextrins include a polysaccharide ether which comprises at least one 2-hydroxybutenyl substituent, wherein the at least one hydroxybutenyl substituent is sulfonated and sulfinated, or disulfonated, and an alkylpolyglycoside ether which comprises at least one 2-hydroxybutenyl substituent, wherein the at least one hydroxybutenyl substituent is sulfonated and sulfinated, or disulfonated. In various embodiments inclusion complexes formed between sulfonated hydroxybutenyl cyclodextrins and one or more of the active agent(s) described herein are contemplated. Methods of preparing cyclodextrins, and cyclodextrin inclusion complexes are found for example in U.S. Patent Publication No: 2004/0054164 and the references cited therein and in U.S. Patent Publication No: 2011/0218173 and the references cited therein.

Pharmacokinetics (PK) and Formulation Attributes

One advantage of the extended (controlled) release oral (GI or transmucosal) formulations described herein is that they can maintain the plasma drug concentration within a targeted therapeutic window for a longer duration than with immediate-release formulations, whether solid dosage forms or liquid-based dosage forms. The high peak plasma levels typically observed for such conventional immediate release formulations will be blunted by the prolonged release of the drug over 1 to 12 hours or longer. In addition, a rapid decline in plasma levels will be avoided since the drug will continually be crossing from the oral cavity into the bloodstream during the length of time of dissolution of the tablet, thus providing plasma pharmacokinetics with a more stable plateau. In addition, the dosage forms described herein may improve treatment safety by minimizing the potentially deleterious side effects due to the reduction of the peaks and troughs in the plasma drug pharmacokinetics, which compromise treatment safety.

In various embodiments the oral transmucosal formulations of the active agent(s) described herein designed to avoid the high peak plasma levels of intravenous and conventional immediate release oral dosage forms by utilizing the mucosa and by independently controlling both tablet disintegration (or erosion) and drug dissolution and release from the tablet over time to provide a safer delivery profile. The oral formulations described herein provide individual, repetitive doses that include a defined amount of the active agent.

An advantage of the bioadhesive oral transmucosal formulations described herein is that they exhibit highly consistent bioavailability and can maintain the plasma drug concentration within a targeted therapeutic window with significantly lower variability for a longer duration than currently available dosage forms, whether solid dosage forms or IV dosage forms. In addition, a rapid decline in plasma levels is avoided since the drug is continually crossing from the oral cavity or GI tract into the bloodstream during the length of time of dissolution of the tablet or longer, thus providing plasma pharmacokinetics with an extended plateau phase as compared to the conventional immediate release oral dosage forms. Further, the dosage forms described herein can improve treatment safety by minimizing the potentially deleterious side effects due to the relative reduction of the peaks and troughs in the plasma drug pharmacokinetics, which compromise treatment safety and is typical of currently available dosage forms.

In various embodiments bioadhesive formulations described herein can be designed to manipulate and control the pharmacokinetic profile of the active agent(s) described herein. As such, the formulations can be adjusted to achieve ‘slow’ disintegration times (and erosion kinetic profiles) and slow drug release and thus enable very prolonged pharmacokinetic profiles that provide sustained drug action. Although such formulations may be designed to still provide a fast onset, they are mostly intended to enable the sustained drug PK and effect while maintaining the other performance attributes of the tablet such as bioadhesion, reproducibility of action, blunted Cₘₐₓ, etc.

The performance and attributes of the bioadhesive transmucosal formulations of this invention are independent of the manufacturing process. A number of conventional, well-established and known in the art processes can be used to manufacture the formulations of the present invention (such as wet and dry granulation, direct compression, etc.) without impacting the dosage form physicochemical properties or in vivo performance.

An illustrative mathematical ratio that demonstrates the prolonged plateau phase of the measured blood plasma levels of the active agent(s) described herein, following administration of the dosage forms of the invention is the term “Optimal Therapeutic Targeting Ratio” or “OTTR”, which represents the average time that the drug is present at therapeutic levels, defined as time within which the drug plasma concentration is maintained above 50% of Cₘₐₓ normalized by the drug's elimination half-life multiplied by the ratio of the Cₘₐₓ obtained in the dosage form of interest over the normalized Cₘₐₓ following IV administration of equivalent doses. In certain embodiments the OTTR can be calculated by the formula:

OTTR=(C^{IV}ₘₐₓ/Cₘₐₓ)×(Dose/Dose^{IV})(Time above 50% of Cₘₐₓ)/(Terminal^{IV} elimination half-life of the drug).

In certain embodiments the OTTR is greater than about 15, or greater than about 20, or greater than about 25, or greater than about 30, or greater than about 40, or greater than about 50.

### Administration

In certain embodiments one or more active agents described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) are administered to a mammal in need thereof, e.g., to a mammal at risk for or suffering from a pathology characterized by reduced sirtuins expression and/or activity, and/or characterized by reduced ADAM10 expression and/or activity, and/or characterized by abnormal processing of amyloid precursor proteins, a mammal at risk for progression of MCI to Alzheimer's disease, and so forth. In certain embodiments the active agent(s) are administered to prevent or delay the onset of a pre-Alzheimer's cognitive dysfunction, and/or to ameliorate one or more symptoms of a pre-Alzheimer's cognitive dysfunction, and/or to prevent or delay the progression of a pre-Alzheimer's condition or cognitive dysfunction to Alzheimer's disease, and/or to promote the processing of amyloid precursor protein (APP) by a non-amyloidogenic pathway. In certain embodiments one or more active agent(s) are administered for the treatment of early stage, mid stage, or late-stage Alzheimer's disease, e.g., to reduce the severity of the disease, and/or to ameliorate one or more symptoms of the disease, and/or to slow the progression of the disease. In certain embodiments, one or more active agents are administered for the treatment or prophylaxis of diabetes and/or metabolic syndrome, and/or to extend lifespan and/or healthspan.

In various embodiments the active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) can be administered by any of a number of routes. Thus, for example they can be administered orally, parenterally, (intravenously (IV), intramuscularly (IM), depo-IM, subcutaneously (SQ), and depo-SQ), sublingually, intranasally (inhalation), intrathecally, transdermally (e.g., via transdermal patch), topically, ionophoretically or rectally. Typically the dosage form is selected to facilitate delivery to the brain (e.g., passage through the blood brain barrier). In this context it is noted that the compounds described herein are readily delivered to the brain. Dosage forms known to those of skill in the art are suitable for delivery of the compound.

In various embodiments the active agent(s) are administered in an amount/dosage regimen sufficient to exert a prophylactically and/or therapeutically useful effect in the absence of undesirable side effects on the subject treated (or with the presence of acceptable levels and/or types of side effects). The specific amount/dosage regimen will vary depending on the weight, gender, age and health of the individual; the formulation, the biochemical nature, bioactivity, bioavailability and the side effects of the particular compound.

In certain embodiments the therapeutically or prophylactically effective amount may be determined empirically by testing the agent(s) in known in vitro and in vivo model systems for the treated disorder. A therapeutically or prophylactically effective dose can be determined by first administering a low dose, and then incrementally increasing until a dose is reached that achieves the desired effect with minimal or no undesired side effects.

In certain embodiments, when administered orally, an administered amount of the agent(s) described herein effective to prevent or delay the onset of a pre-Alzheimer's cognitive dysfunction, and/or to ameliorate one or more symptoms of a pre-Alzheimer's cognitive dysfunction, and/or to prevent or delay the progression of a pre-Alzheimer's condition or cognitive dysfunction to Alzheimer's disease, and/or to promote the processing of amyloid precursor protein (APP) by a non-amyloidogenic pathway, and/or to treat or prevent AD ranges from about 0.1 mg/day to about 500 mg/day or about 1,000 mg/day, or from about 0.1 mg/day to about 200 mg/day, for example, from about 1 mg/day to about 100 mg/day, for example, from about 5 mg/day to about 50 mg/day. In some embodiments, the subject is administered the compound at a dose of about 0.05 to about 0.50 mg/kg, for example, about 0.05 mg/kg, 0.10 mg/kg, 0.20 mg/kg, 0.33 mg/kg, 0.50 mg/kg. It is understood that while a patient may be started at one dose, that dose may be varied (increased or decreased, as appropriate) over time as the patient's condition changes. Depending on outcome evaluations, higher doses may be used. For example, in certain embodiments, up to as much as 1000 mg/day can be administered, e.g., 5 mg/day, 10 mg/day, 25 mg/day, 50 mg/day, 100 mg/day, 200 mg/day, 300 mg/day, 400 mg/day, 500 mg/day, 600 mg/day, 700 mg/day, 800 mg/day, 900 mg/day or 1000 mg/day.

In various embodiments, active agent(s) described herein can be administered parenterally, for example, by IV, IM, depo-IM, SC, or depo-SC. In certain embodiments when administered parenterally, a therapeutically effective amount of about 0.5 to about 100 mg/day, preferably from about 5 to about 50 mg daily can be delivered. When a depot formulation is used for injection once a month or once every two weeks, the dose in certain embodiments can be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

In various embodiments, the active agent(s) described herein can be administered sublingually. In some embodiments, when given sublingually, the compounds and/or analogs thereof can be given one to four times daily in the amounts described above for IM administration.

In various embodiments, the active agent(s) described herein can be administered intranasally. When given by this route, the appropriate dosage forms are a nasal spray or dry powder, as is known to those skilled in the art. In certain embodiments, the dosage of compound and/or analog thereof for intranasal administration is the amount described above for IM administration.

In various embodiments, the active agent(s) described herein can be administered intrathecally. When given by this route the appropriate dosage form can be a parenteral dosage form as is known to those skilled in the art. In certain embodiments, the dosage of compound and/or analog thereof for intrathecal administration is the amount described above for IM administration.

In certain embodiments, the active agent(s) described herein can be administered topically. When given by this route, the appropriate dosage form is a cream, ointment, or patch. When administered topically, the dosage is from about 1.0 mg/day to about 200 mg/day. Because the amount that can be delivered by a patch is limited, two or more patches may be used. The number and size of the patch is not important as long as a therapeutically effective amount of compound be delivered as is known to those skilled in the art. The compound can be administered rectally by suppository as is known to those skilled in the art. In certain embodiments, when administered by suppository, the therapeutically effective amount is from about 1.0 mg to about 500 mg.

In various embodiments, the active agent(s) described herein can be administered by implants as is known to those skilled in the art. When administering the compound by implant, the therapeutically effective amount is the amount described above for depot administration.

In various embodiments, the active agent(s) described herein thereof can be enclosed in multiple or single dose containers. The enclosed agent(s) can be provided in kits, for example, including component parts that can be assembled for use. For example, an active agent in lyophilized form and a suitable diluent may be provided as separated components for combination prior to use. A kit may include an active agent and a second therapeutic agent for co-administration. The active agent and second therapeutic agent may be provided as separate component parts. A kit may include a plurality of containers, each container holding one or more unit dose of the compounds. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration, e.g., as described herein.

In various embodiments the dosage forms can be administered to the subject 1, 2, 3, or 4 times daily. In certain embodiments it is preferred that the compound be administered either three or fewer times, more preferably once or twice daily. In certain embodiments, it is preferred that the agent(s) be administered in oral dosage form.

It should be apparent to one skilled in the art that the exact dosage and frequency of administration will depend on the particular condition being treated, the severity of the condition being treated, the age, weight, general physical condition of the particular patient, and other medication the individual may be taking as is well known to administering physicians who are skilled in this art.

While the compositions and methods are described herein with respect to use in humans, they are also suitable for animal, e.g., veterinary use. Thus certain organisms (subjects) contemplated herein include, but are not limited to humans, non-human primates, canines, equines, felines, porcines, ungulates, largomorphs, and the like.

The foregoing formulations and administration methods are intended to be illustrative and not limiting. It will be appreciated that, using the teaching provided herein, other suitable formulations and modes of administration can be readily devised.

### Combination Therapies

In certain embodiments, the active agent(s) described herein (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) can be used in combination with other therapeutic agents or approaches used to treat or prevent diseases characterized by amyloid deposits in the brain, including MCI and/or AD. Accordingly, in certain embodiments, a pharmaceutical composition comprising at least active agent described herein (e.g., alaproclate and other compounds described herein, or a tautomer or stereoisomer thereof, or pharmaceutically acceptable salts or solvate of said alaproclate and other compounds, said stereoisomer, or said tautomer, or an analogue, derivative, or prodrug thereof) one together with at least one additional therapeutic agent, and a pharmaceutically acceptable carrier or diluent is contemplated. In certain embodiments a therapeutic or prophylactic method comprising administering at least active agent described herein in conjunction with at least one additional therapeutic agent is contemplated.

In certain embodiments non-limiting examples of additional therapeutic agents include, but are not limited to disulfiram and/or analogues thereof, honokiol and/or analogues thereof, tropisetron and/or analogues thereof, nimetazepam and/or analogues thereof (see, e.g., U.S. Ser. No. 13/213,960 (U.S. Patent Publication No: US-2012-0071468-A1), and PCT/US2011/048472 (PCT Publication No: WO 2012/024616) which are incorporated herein by reference for the compounds described therein), tropinol-esters and/or related esters and/or analogues thereof (see, e.g., U.S. Ser. No. 61/514,381, which is incorporated herein by reference for the compounds described herein), TrkA kinase inhibitors (e.g., ADDN-1351) and/or analogues thereof (see, e.g., U.S. Ser. No. 61/525,076, which is incorporated herein by reference for the compounds described therein), D2 receptor agonists and alpha1-adrenergic receptor antagonists, and APP-specific BACE Inhibitors (ASBIs) as described and/or claimed in U.S. Ser. No. 61/728,688, filed on Nov. 20, 2012 which is incorporated herein by reference for the active agents described herein including, but not limited to galangin, a galangin prodrug, rutin, a rutin prodrug, and other flavonoids and flavonoid prodrugs as described or claimed therein, other BACE inhibitors such as MK-8931 in phase 3 clinical testing and AZD-3839 in phase2-3 clinical testing.

Non-limiting examples of additional therapeutic agents include drugs selected from the group consisting of: (a) drugs useful for the treatment of Alzheimer's disease and/or drugs useful for treating one or more symptoms of Alzheimer's disease, (b) drugs useful for inhibiting the synthesis Aβ, and (c) drugs useful for treating neurodegenerative diseases. Additional non-limiting examples of additional therapeutic agents for use in combination with the compounds (e.g., alaproclate and other compounds) described herein include drugs useful for the treatment, prevention, delay of onset, amelioration of any pathology associated with Aβ and/or a symptom thereof. Non-limiting examples of pathologies associated with Aβ include: Alzheimer's disease, Down's syndrome, Parkinson's disease, memory loss, memory loss associated with Alzheimer's disease, memory loss associated with Parkinson's disease, attention deficit symptoms, attention deficit symptoms associated with Alzheimer's disease, Parkinson's disease, and/or Down's syndrome, dementia, stroke, microgliosis and brain inflammation, pre-senile dementia, senile dementia, dementia associated with Alzheimer's disease, Parkinson's disease, and/or Down's syndrome, progressive supranuclear palsy, cortical basal degeneration, neurodegeneration, olfactory impairment, olfactory impairment associated with Alzheimer's disease, Parkinson's disease, and/or Down's syndrome, β-amyloid angiopathy, cerebral amyloid angiopathy, hereditary cerebral hemorrhage, mild cognitive impairment (“MCI”), glaucoma, amyloidosis, type II diabetes, hemodialysis complications (from β.sub.2 microglobulins and complications arising therefrom in hemodialysis patients), scrapie, bovine spongiform encephalitis, traumatic brain injury (“TBI”), and Creutzfeld-Jakob disease, comprising administering to said patient at least one active agent described herein, or a tautomer or isomer thereof or pharmaceutically acceptable salt or solvate of said compound or said tautomer, in an amount effective to inhibit said pathology or pathologies and/or to promote lifespan and/or healthspan.

In certain embodiments such additional therapeutic agents include, but are not limited to acetylcholinesterase inhibitors (including without limitation, e.g., (−)-phenserine enantiomer, tacrine, ipidacrine, galantamine, donepezil, icopezil, zanapezil, rivastigmine, huperzine A, phenserine, physostigmine, neostigmine, pyridostigmine, ambenonium, demarcarium, edrophonium, ladostigil and ungeremine); NMDA receptor antagonist (including without limitations e.g., Memantine); muscarinic receptor agonists (including without limitation, e.g., Talsaclidine, AF-102B, AF-267B (NGX-267)); nicotinic receptor agonists (including without limitation, e.g., Ispronicline (AZD-3480)); beta-secretase inhibitors (including without limitations e.g., thiazolidinediones, including rosiglitazone and pioglitazone); gamma-secretase inhibitors (including without limitation, e.g., semagacestat (LY-450139), MK-0752, E-2012, BMS-708163, PF-3084014, begacestat (GSI-953), and NICS-15); inhibitors of Aβ aggregation (including without limitation, e.g., Clioquinol (PBT1), PBT2, tramiprosate (homotaurine), Scyllo-inositol (a.k.a., scyllo-cyclohexanehexol, AZD-103 and ELND-005), passive immunotherapy with Aβ fragments (including without limitations e.g., Bapineuzemab) and Epigallocatechin-3-gallate (EGCg)); anti-inflammatory agents such as cyclooxygenase II inhibitors; anti-oxidants such as Vitamin E and ginkolides; immunological approaches, such as, for example, immunization with Aβ peptide or administration of anti-Aβ peptide antibodies; statins; and direct or indirect neurotrophic agents such as Cerebrolysin™, AIT-082 (Emilieu, 2000, Arch. Neurol. 57:454), Netrin (Luorenco (2009) Cell Death Differ., 16: 655-663), Netrin mimetics, NGF, NGF mimetics, BDNF and other neurotrophic agents of the future, agents that promote neurogenesis e.g. stem cell therapy. Further pharmacologic agents useful in the treatment or prevention diseases characterized by amyloid deposits in the brain, including MCI and/or AD, are described, e.g., in Mangialasche, et al. (2010) Lancet Neurol., 9:702-716.

In certain embodiments, additional non-limiting examples of additional therapeutic agents for use in combination with compounds described herein include: muscarinic antagonists (e.g., m₁ agonists (such as acetylcholine, oxotremorine, carbachol, or McNa343), or m₂ antagonists cholinesterase inhibitors (e.g., acetyl- and/or butyrylchlolinesterase inhibitors such as donepezil (Aricept®), galantamine (Razadyne®), and rivastigimine (Exelon®); N-methyl-D-aspartate receptor antagonists (e.g., NAMENDA® (memantine HCl); combinations of cholinesterase inhibitors and N-methyl-D-aspartate receptor antagonists; gamma secretase modulators; gamma secretase inhibitors; non-steroidal anti-inflammatory agents; anti-inflammatory agents that can reduce neuroinflammation; anti-amyloid antibodies (such as bapineuzemab, Wyeth/Elan); vitamin E; nicotinic acetylcholine receptor agonists; CB1 receptor inverse agonists or CB1 receptor antagonists; antibiotics; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Tau kinase inhibitors (e.g., GSK3beta inhibitors, cdk5 inhibitors, or ERK inhibitors); Tau aggregation inhibitors (e.g., REMBER®; RAGE inhibitors (e.g., TTP 488 (PF-4494700)); anti-Aβ vaccine; APP ligands; agents that upregulate insulin, cholesterol lowering agents such as HMG-CoA reductase inhibitors (for example, statins such as Atorvastatin, Fluvastatin, Lovastatin, Mevastatin, Pitavastatin, Pravastatin, Rosuvastatin, Simvastatin) and/or cholesterol absorption inhibitors (such as Ezetimibe), or combinations of HMG-CoA reductase inhibitors and cholesterol absorption inhibitors (such as, for example, VYTORIN®); fibrates (such as, for example, clofibrate, Clofibride, Etofibrate, and Aluminium Clofibrate); combinations of fibrates and cholesterol lowering agents and/or cholesterol absorption inhibitors; nicotinic receptor agonists; niacin; combinations of niacin and cholesterol absorption inhibitors and/or cholesterol lowering agents (e.g., SIMCOR® (niacin/simvastatin, available from Abbott Laboratories, Inc.); LXR agonists; LRP mimics; H3 receptor antagonists; histone deacetylase inhibitors; hsp90 inhibitors; 5-HT4 agonists (e.g., PRX-03140 (Epix Pharmaceuticals)); 5-HT6 receptor antagonists; mGluR1 receptor modulators or antagonists; mGluR5 receptor modulators or antagonists; mGluR2/3 antagonists; Prostaglandin EP2 receptor antagonists; PAI-1 inhibitors; agents that can induce Abeta efflux such as gelsolin; Metal-protein attenuating compound (e.g., PBT2); and GPR3 modulators; and antihistamines such as Dimebolin (e.g., DIMEBON®, Pfizer).

Accordingly certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is disulfiram and/or analogues thereof (see, e.g., U.S. Ser. No. 13/213,960 (U.S. Patent Publication No: US-2012-0071468-A1), and PCT/US2011/048472 (PCT Publication No: WO 2012/024616)).

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is honokiol and/or analogues thereof (see, e.g., U.S. Ser. No. 13/213,960 (U.S. Patent Publication No: US-2012-0071468-A1), and PCT/US2011/048472 (PCT Publication No: WO 2012/024616)).

Certain embodiments provide a pharmaceutical composition comprising an effective amount of one or more of alaproclate or other compounds described herein and an additional therapeutic agent, and/or a method of treatment or prophylaxis comprising administration of one or more alaproclate or other compounds described herein in conjunction with an additional therapeutic agent where the therapeutic agent in the formulation and/or method is tropisetron and/or analogues thereof (see, e.g., U.S. Ser. No. 13/213,960 (U.S. Patent Publication No: US-2012-0071468-A1), and PCT/US2011/048472 (PCT Publication No: WO 2012/024616)).

Certain embodiments provide a pharmaceutical composition comprising an effective amount of one or more alaproclate or other compounds described herein and an additional therapeutic agent, and/or a method of treatment or prophylaxis comprising administration of one or more alaproclate or other compounds described herein in conjunction with an additional therapeutic agent where the therapeutic agent in the formulation and/or method is tropisetron.

Certain embodiments provide a pharmaceutical composition comprising an effective amount of one or more alaproclate or other compounds described herein and an additional therapeutic agent, and/or a method of treatment or prophylaxis comprising administration of one or more alaproclate or other compounds described herein in conjunction with an additional therapeutic agent where the therapeutic agent in the formulation and/or method is nimetazepam and/or analogues thereof (see, e.g., U.S. Ser. No. 13/213,960 (U.S. Patent Publication No: US-2012-0071468-A1), and PCT/US2011/048472 (PCT Publication No: WO 2012/024616)).

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is a tropinol ester or related ester (see, e.g., U.S. Ser. No. 61/514,381).

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is a TrkA kinase inhibitor (e.g., ADDN-1351) and/or analogues thereof (see, e.g., U.S. Ser. No. 61/525,076).

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is a D2 receptor agonists and/or an alpha1-adrenergic receptor antagonists.

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is an ASBIs as described and/or claimed in U.S. Ser. No. 61/728,688, filed on Nov. 20, 2012 which is incorporated herein by reference for the active agents described herein including, but not limited to galangin, a galangin prodrug, rutin, a, and other flavonoids as described or claimed therein.

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is one or more cholinesterase inhibitors (e.g., acetyl- and/or butyrylchlolinesterase inhibitors).

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is one or more muscarinic antagonists (e.g., m₁ agonists or m₂ antagonists).

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is one or more hydantoins (e.g., as described in PCT application No: PCT/US2014/016100 and in U.S. patent application Ser. No. 14/179,310).

Certain embodiments provide a pharmaceutical composition comprising an effective amount of one or more of alaproclate or other compounds described herein and an additional therapeutic agent, and/or a method of treatment or prophylaxis comprising administration of one or more of alaproclate or other compounds described herein in conjunction with an additional therapeutic agent where the therapeutic agent in the formulation and/or method is one or more compounds selected from the group consisting of cholinesterase inhibitors (such as, for example, (.+−.)-2,3-dihydro-5,6-dimethoxy-2-[[1-(phenylmethyl)-4-piperidinyl]methy-1]-1H-inden-1-one hydrochloride, i.e, donepezil hydrochloride, available as the ARICEPT® brand of donepezil hydrochloride), N-methyl-D-aspartate receptor inhibitors (such as, for example, Namenda® (memantine HCl)); anti-amyloid antibodies (such as bapineuzumab, Wyeth/Elan), gamma secretase inhibitors, gamma secretase modulators, and beta secretase inhibitors.

### Additional Indications.

Aging/Lifespan/Healthspan.

Preliminary studies with resveratrol, a possible SIRT1 activator, have led some scientists to speculate that resveratrol may extend lifespan (Patel et al. (2005) Neurobiol. Aging. 26(7): 995-1000). Further experiments. showed that resveratrol-mimicking drugs such as SRT1720 could extend the lifespan of obese mice by 44% (Tippmann et al. (2009) FASEB J. 23(6): 1643-1654).

Cell culture research into the behaviour of the human sirtuin SIRT1 shows that it behaves like the yeast sirtuin Sir2: SIRT2 assists in the repair of DNA and regulates genes that undergo altered expression with age (Costa et al. (2005) Trends Neurosci. 28(8): 429-435). Adding resveratrol to the diet of mice inhibit gene expression profiles associated with muscle aging and age-related cardiac dysfunction (Xiao et al. (2009) Int. J. Physiol, Pathophysiol, & Pharmacol., 1(2): 192-202).

A study performed on transgenic mice overexpressing SIRT6, showed an increased lifespan of about 15% in males. The transgenic males displayed lower serum levels of insulin-like growth factor 1 (IGF1) and changes in its metabolism, which may have contributed to the increased lifespan (Heneka et al. (2010) J. Neural Transm. 117(8): 919-947).

Additionally, it has been demonstrated that brain-specific Sirt1-overexpressing (BRASTO) transgenic mice show significant life span extension in both males and females, and aged BRASTO mice exhibit phenotypes consistent with a delay in aging. These phenotypes were mediated by enhanced neural activity specifically in the dorsomedial and lateral hypothalamic nuclei through increased orexin type 2 receptor (Ox2r) expression. Nk2 homeobox 1 (Nkx2-1) as a partner of Sirt1 that upregulates Ox2r transcription and colocalizes with Sirt1 in the DMH and LH. DMH/LH-specific knockdown of Sirt1, Nkx2-1, or Ox2r and DMH-specific Sirt1 overexpression further supported the role of Sirt1/Nkx2-1/Ox2r-mediated signaling for longevity-associated phenotypes. The findings indicate the importance of DMH/LH-predominant Sirt1 activity in the regulation of aging and longevity in mammals (Satoh et al. (2013) Cell Metabolism, 18(3): 416-430).

Accordingly, it is believed that the active agents described herein (e.g., agents that upregulate SirT1 expression) are useful agents for the promotion of increased lifespan and/or healthspan.

Diabetes and/or Metabolic Syndrome.

Insulin resistance and subclinical atherosclerosis are associated with SIRT1 downregulation in monocytes. Glucotoxicity and lypotoxicity play a relevant role in quenching SirT1 expression. Metabolic syndrome is increasingly prevalent in the general population. Excess caloric intake and nutrient availability are the obvious culprits that lead to obesity and insulin resistance. In turn, metabolic syndrome predisposes to early atherosclerosis and cardiovascular morbidity (Bertoni et al. (2007) Diabetes Care 30: 2951-2956). The evolutionary conserved silent information regulator 2 (SIR2) is a NAD⁺-dependent deacetylase that regulates life span in response to caloric restriction in many organisms. Mammalian homologues of SIR2 comprise a family of seven proteins termed Sirtuins (SIRT1-SIRT7), which are implicated in metabolic processes and stress resistance (Imai et al. (2000) Nature 403: 795-800; Guarente (2006) Nature 444: 868-874). Caloric restriction extends life span in a variety of organisms through induction of SIRT (Westphal et al. (2007) Trends Biochem. Sci. 32: 555-560). In mammals, SIRT1 deacetylates many key transcription factors and cofactors, such as the tumor suppressor p53, forkhead box class O (FOXO) proteins (Motta et al. (2004) Cell, 116: 551-563), peroxisome proliferator-activated receptor-γ coactivator-1α (PGC-1α) (Rodgers et al. (2005) Nature, 434: 113-118), and nuclear factor-κB (Yeung et al. (2004) EMBO J. 23: 2369-2380). These specific actions may affect cellular pathways involved in glucose homeostasis. The effects of SIRT appear to be beneficial, as they trigger metabolic changes similar to those observed in caloric restriction. Indeed, calorie restriction increases the levels of SIRT1 in the liver and muscle, which are key insulin-sensitive organs (Cohen et al. (2004) Science, 305: 390-392). Moreover, SIRT1^{-/-} mice are insensitive to the metabolic effects of caloric restriction (Chen et al. (2005) Science 310: 1641).

In light of these observations, SIRTs have been proposed as a possible target for the treatment of metabolic syndrome (Guarente (2006) Nature 444: 868-874; Westphal et al. (2007) Trends Biochem. Sci. 32: 555-560; Jiang (2008) Biochem. Biophys. Res. Commun. 373: 341-344). In white adipose tissue, SIRT1 was shown to inhibit adipogenesis and to reduce fat storage in differentiated cells (Picard et al. (2004) Nature, 429: 771-776). In parallel, pancreatic β-cells were found to be highly enriched in SIRT4: knocking out this SIRT in insulinoma cells and in mice triggers insulin hypersecretion (Haigis et al. (2006) Cell, 126: 941-954; Ahuja et al. (2007) J. Biol. Chem. 282: 33583-33592).

In an effort to determine that SIRTs are altered in the setting of metabolic syndrome, a well-known condition of insulin resistance experiments have been performed to determine whether insulin resistance and metabolic syndrome and its components are associated with altered SIRT gene and protein expression in circulating peripheral blood mononuclear cells (PBMCs) (Vigili de Kreutzenberg et al. (2010) Diabetes, 59(4): 1006-1015). Monocytes play a major role in pathogenetic processes linked to metabolic syndrome, such as inflammation of the adipose tissue and development of the atherosclerotic plaque (Libby et al. (1996) Curr. Opin. Lipidol. 17: 330-335; Odegaard and Chawla (2008) Nat. Clin. Pract. Endocrinol. Metab. 4: 619-626).

A major result from the in vivo study was that gene and protein expression of SirT1 in PBMCs was significantly reduced in relation to insulin resistance and metabolic syndrome. This result was corroborated by the direct correlation between SirT1 expression and a dynamic measure of insulin sensitivity, as well as by the correlation between SIRT1 expression and the number of metabolic syndrome components (Vigili de Kreutzenberg et al. (2010) Diabetes, 59(4): 1006-1015).

The link between SirT1 and glucose homeostasis was substantiated by the SIRT1 downregulation observed in subjects with pre-diabetes, compared with subjects with normal glucose regulation. Thus, the expression of SIRT1 in PBMCs appears as a novel marker of insulin resistance, metabolic syndrome, and pre-diabetes (Id.). The data suggested that SIRT1 expression is decreased in subjects who are insulin resistant, specifically in those who are glucose intolerant, and particularly in those with several components of metabolic syndrome (Id.).

Accordingly, it is believed that the active agents described herein (e.g., agents that upregulate SirT1 expression) are useful agents for the prophylaxis and/or therapeutic treatment of diabetes and/or metabolic syndrome.

### Assay Systems to Evaluate Modulation of APP Processing by Active Agents.

Without being bound to a particular theory, it is believed that the active agent(s) described herein (e.g., alaproclate and other alaproclate “related” active agents described herein) upregulate SirT1 and/or ADAM10, and promote processing of APP by the nonamyloidogenic pathway and/or reduce or inhibit processing of APP by the amyloidogenic pathway. In the nonamyloidogeic pathway, APP is first cleaved by α-secretase within the Aβ sequence, releasing the APPsα ectodomain (“sAPPα”). In contrast, the amyloidogenic pathway is initiated when β-secretase cleaves APP at the amino terminus of the Aβ, thereby releasing the APPsβ ectodomain (“sAPPβ”). APP processing by the nonamyloidogenic and amyloidogenic pathways is known in the art and reviewed, e.g., by Xu (2009) J Alzheimers Dis., 16(2): 211-224, and De Strooper, et al. (2010 Nat Rev Neurol 6(2): 99-107.

One method to evaluate the efficacy of the active agent(s) is to determine a reduction or elimination in the level of APP processing by the amyloidogenic pathway, e.g., a reduction or elimination in the level of APP processing by β-secretase cleavage in response to the administration of the agent(s) of interest. Assays for determining the extent of APP cleavage at the β-secretase cleavage site are well known in the art. Illustrative assays are described, for example, in U.S. Pat. Nos. 5,744,346 and 5,942,400. Kits for determining the presence and levels in a biological sample of sAPPα and sAPPβ, as well as APPneo and Aβ commercially available, e.g., from PerkinElmer.

Other illustrative assays that can be used to demonstrate the activity of the activity of the agent(s) described herein are described, for example, in WO 2000/017369, WO 2000/0003819, and U.S. Pat. Nos. 5,942,400 and 5,744,346.

Cellular Assays

Numerous cell-based assays can be used to evaluate the activity of agent(s) of interest on relative alpha-secretase activity and/or beta-secretase activity and/or processing of APP to release amyloidogenic versus non-amyloidogenic Aβ oligomers. Contact of an APP substrate with an alpha-secretase and/or beta-secretase enzyme within the cell and in the presence or absence of the agent(s) can be used to demonstrate alpha-secretase promoting and/or beta-secretase inhibitory activity of the agent(s). Preferably, the assay in the presence of the agent(s) provides at least about 30%, most preferably at least about 50% inhibition of the enzymatic activity, as compared with a non-inhibited control.

In one embodiment, cells that naturally express alpha-secretase and/or beta-secretase are used. Alternatively, cells are modified to express a recombinant alpha-secretase and/or beta-secretase or synthetic variant enzymes, as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the alpha-secretase and/or beta-secretase APP cleavage sites can be used, provided that the expressed APP is permitted to contact the enzyme and enzymatic cleavage activity can be analyzed.

Human cell lines that normally process Aβ from APP provide a useful means to assay inhibitory activities of the agent(s). Production and release of Aβ and/or other cleavage products into the culture medium can be measured, for example by immunoassay, such as Western blot or enzyme-linked immunoassay (EIA) such as by ELISA.

Cells expressing an APP substrate and an active alpha-secretase and/or beta-secretase can be incubated in the presence of the agents to demonstrate relative enzymatic activity of the alpha-secretase and/or beta-secretase as compared with a control. Relative activity of the alpha-secretase to the beta-secretase can be measured by analysis of one or more cleavage products of the APP substrate. For example, inhibition of beta-secretase activity against the substrate APP would be expected to decrease release of specific beta-secretase induced APP cleavage products such as Aβ (e.g., Aβ40 or Aβ42), sAPPβ and APPneo. Promotion or enhancement of alpha-secretase activity against the substrate APP would be expected to increase release of specific alpha-secretase induced APP cleavage products such as sAPPα and p3 peptide.

Although both neural and non-neural cells process and release Aβ, levels of endogenous beta-secretase activity are low and often difficult to detect by EIA. The use of cell types known to have enhanced beta-secretase activity, enhanced processing of APP to Aβ, and/or enhanced production of Aβ are therefore preferred. For example, transfection of cells with the Swedish Mutant form of APP (APP-SW); with the Indiana Mutant form (APP-IN); or with APP-SW-IN provides cells having enhanced beta-secretase activity and producing amounts of Aβ that can be readily measured.

In such assays, for example, the cells expressing APP, alpha-secretase and/or beta-secretase are incubated in a culture medium under conditions suitable for alpha-secretase and/or beta-secretase enzymatic activity at its cleavage site on the APP substrate. On exposure of the cells to the agent(s), the amount of Aβ released into the medium and/or the amount of CTF99 fragments of APP in the cell lysates is reduced as compared with the control. The cleavage products of APP can be analyzed, for example, by immune reactions with specific antibodies, as discussed above.

In certain embodiments, preferred cells for analysis of alpha-secretase and/or beta-secretase activity include primary human neuronal cells, primary transgenic animal neuronal cells where the transgene is APP, and other cells such as those of a stable 293 cell line expressing APP, for example, APP-SW.

### In Vivo Assays: Animal Models

Various animal models can be used to analyze the activity of agent(s) of interest on relative alpha-secretase and/or beta-secretase activity and/or processing of APP to release Aβ. For example, transgenic animals expressing APP substrate, alpha-secretase and/or beta-secretase enzyme can be used to demonstrate inhibitory activity of the agent(s). Certain transgenic animal models have been described, for example, in U.S. Pat. Nos. 5,877,399; 5,612,486; 5,387,742; 5,720,936; 5,850,003; 5,877,015, and 5,811,633, and in Games et al. (1995) Nature 373: 523. Preferred are animals that exhibit characteristics associated with the pathophysiology of AD. Administration of the agent(s) to the transgenic mice described herein provides an alternative method for demonstrating the inhibitory activity of the agent(s). Administration of the agent(s) in a pharmaceutically effective carrier and via an administrative route that reaches the target tissue in an appropriate therapeutic amount is also preferred.

Inhibition of beta-secretase mediated cleavage of APP at the beta-secretase cleavage site and of Aβ release can be analyzed in these animals by measure of cleavage fragments in the animal's body fluids such as cerebral fluid or tissues. Likewise, promotion or enhancement of alpha-secretase mediated cleavage of APP at the alpha-secretase cleavage site and of release of sAPPα can be analyzed in these animals by measure of cleavage fragments in the animal's body fluids such as cerebral fluid or tissues. In certain embodiments, analysis of brain tissues for Aβ deposits or plaques is preferred.

On contacting an APP substrate with an alpha-secretase and/or beta-secretase enzyme in the presence of the agent(s) under conditions sufficient to permit enzymatic mediated cleavage of APP and/or release of Aβ from the substrate, desirable agent(s) are effective to reduce beta-secretase-mediated cleavage of APP at the beta-secretase cleavage site and/or effective to reduce released amounts of Aβ. The agent(s) are also preferably effective to enhance alpha-secretase-mediated cleavage of APP at the alpha-secretase cleavage site and to increase released amounts of sAPPα. Where such contacting is the administration of the agent(s) to an animal model, for example, as described above, the agent(s) is effective to reduce Aβ deposition in brain tissues of the animal, and to reduce the number and/or size of beta amyloid plaques. Where such administration is to a human subject, the agent(s) is effective to inhibit or slow the progression of disease characterized by enhanced amounts of Aβ, to slow the progression of AD in the, and/or to prevent onset or development of AD in a patient at risk for the disease. Alaproclate and analogs can be tested in in vivo models for their ability to modulate SirT1 levels and increase sAPPα. The ability of these molecules to improve memory in these mouse models can be determined.

Methods of Monitoring Clinical Efficacy

In various embodiments, the effectiveness of treatment can be determined by comparing a baseline measure of a parameter of disease before administration of the agent(s) (e.g., alaproclate and other alaproclate “related” agents described herein described herein, or a tautomer(s) or stereoisomer(s) thereof, or pharmaceutically acceptable salts or solvates of said alaproclate and other alaproclate “related” agents, said stereoisomer(s), or said tautomer(s), or analogues, derivatives, or prodrugs thereof) is commenced to the same parameter one or more time points after the agent(s) or analog has been administered.

Illustrative parameters include, for example, measurements of expression and/or activity of SirT1 and/or ADAM10. Such assays are illustrated herein in the Examples.

Another illustrative parameter that can be measured is a biomarker (e.g., a peptide oligomer) of APP processing. Such biomarkers include, but are not limited to increased levels of sAPPα, p3 (Aβ17-42 or Aβ17-40), sAPPβ, soluble Aβ40, and/or soluble Aβ42 in the blood, plasma, serum, urine, mucous or cerebrospinal fluid (CSF). Detection of increased levels of sAPPα and/or p3, and decreased levels of sAPPβ and/or APPneo is an indicator that the treatment is effective. Conversely, detection of decreased levels of sAPPα and/or p3, and/or increased levels of sAPPβ, APPneo, Tau or phospho-Tau (pTau) is an indicator that the treatment is not effective.

Another parameter to determine effectiveness of treatment is the level of amyloid plaque deposits in the brain. Amyloid plaques can be determined using any method known in the art, e.g., as determined by CT, PET, PIB-PET and/or MRI. Administration of the agent(s)) (e.g., alaproclate and other alaproclate “related” agents described herein, or a tautomer(s) or stereoisomer(s) thereof, or pharmaceutically acceptable salts or solvates of said alaproclate and other alaproclate “related” agents, said stereoisomer(s), or said tautomer(s), or analogues, derivatives, or prodrugs thereof) can result in a reduction in the rate of plaque formation, and even a refraction or reduction of plaque deposits in the brain. Effectiveness of treatment can also be determined by observing a stabilization and/or improvement of cognitive abilities of the subject. Cognitive abilities can be evaluated using any art-accepted method, including for example, Clinical Dementia Rating (CDR), the mini-mental state examination (MMSE) or Folstein test, evaluative criteria listed in the DSM-IV (Diagnostic and Statistical Manual of Mental Disorders, Fourth Edition) or DSM-V, and the like.

Clinical efficacy can be monitored using any method known in the art. Measurable biomarkers to monitor efficacy include, but are not limited to, monitoring blood, plasma, serum, urine, mucous or cerebrospinal fluid (CSF) levels of sAPPα, sAPPβ, Aβ42, Aβ40, APPneo and p3 (e.g., Aβ17-42 or Aβ17-40). Detection of increased levels of sAPPα and/or p3, and decreased levels of sAPPβ and/or APPneo are indicators that the treatment or prevention regime is efficacious. Conversely, detection of decreased levels of sAPPα and/or p3, and increased levels of sAPPβ and/or APPneo are indicators that the treatment or prevention regime is not efficacious. Other biomarkers include Tau and phospho-Tau (pTau). Detection of decreased levels of Tau and pTau are indicators that the treatment or prevention regime is efficacious.

Efficacy can also be determined by measuring amyloid plaque load in the brain. The treatment or prevention regime is considered efficacious when the amyloid plaque load in the brain does not increase or is reduced. Conversely, the treatment or prevention regime is considered inefficacious when the amyloid plaque load in the brain increases. Amyloid plaque load can be determined using any method known in the art, e.g., including CT, PET, PIB-PET and/or MRI.

Efficacy can also be determined by measuring the cognitive abilities of the subject. Cognitive abilities can be measured using any method known in the art. Illustrative tests include assigning a Clinical Dementia Rating (CDR) score or applying the mini mental state examination (MMSE) (Folstein, et al., J. Psychiatric Res. 12(3): 189-198). Subjects who maintain the same score or who achieve an improved score, e.g., when applying the CDR or MMSE, indicate that the treatment or prevention regime is efficacious. Conversely, subjects who receive a score indicating diminished cognitive abilities, e.g., when applying the CDR or MMSE, indicate that the treatment or prevention regime has not been efficacious.

In certain embodiments, the monitoring methods can entail determining a baseline value of a measurable biomarker or parameter (e.g., amyloid plaque load or cognitive abilities) in a subject before administering a dosage of the agent(s), and comparing this with a value for the same measurable biomarker or parameter after treatment.

In other methods, a control value (e.g., a mean and standard deviation) of the measurable biomarker or parameter is determined for a control population. In certain embodiments, the individuals in the control population have not received prior treatment and do not have AD, MCI, nor are at risk of developing AD or MCI. In such cases, if the value of the measurable biomarker or clinical parameter approaches the control value, then treatment is considered efficacious. In other embodiments, the individuals in the control population have not received prior treatment and have been diagnosed with AD or MCI. In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation/ANOVA) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (e.g., more than a standard deviation) is an indicator that treatment should be resumed in the subject.

In certain embodiments the tissue sample for analysis is typically blood, plasma, serum, urine, mucous or cerebrospinal fluid from the subject.

### Kits.

In various embodiments, the active agent(s) (e.g., alaproclate and other alaproclate “related” agents) described herein thereof can be enclosed in multiple or single dose containers. The enclosed agent(s) can be provided in kits, for example, including component parts that can be assembled for use. For example, an active agent in lyophilized form and a suitable diluent may be provided as separated components for combination prior to use. A kit may include an active agent and a second therapeutic agent for co-administration. The active agent and second therapeutic agent may be provided as separate component parts. A kit may include a plurality of containers, each container holding one or more unit dose of the compounds. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration, e.g., as described herein.

In certain embodiments, a kit is provided where the kit comprises one or more alaproclate and other alaproclate “related” agents described herein, or a tautomer or stereoisomer thereof, or pharmaceutically acceptable salt or solvate of said compound, said stereoisomer, or said tautomer, preferably provided as a pharmaceutical composition and in a suitable container or containers and/or with suitable packaging; optionally one or more additional active agents, which if present are preferably provided as a pharmaceutical composition and in a suitable container or containers and/or with suitable packaging; and optionally instructions for use, for example written instructions on how to administer the compound or compositions.

In another embodiment, a kit is provided that comprises a single container or multiple containers: (a) a pharmaceutically acceptable composition comprising one or more compounds described herein (e.g., alaproclate and other alaproclate “related” agents), or a tautomer or stereoisomer thereof, or pharmaceutically acceptable salt or solvate of said compound, said stereoisomer, or said tautomer, optionally a pharmaceutically acceptable composition comprising one or more additional therapeutic agents; and optionally instructions for use their use. The kit may optionally comprise labeling (e.g., instructional materials) appropriate to the intended use or uses.

As with any pharmaceutical product, the packaging material(s) and/or container(s) are designed to protect the stability of the product during storage and shipment. In addition, the kits can include instructions for use or other informational material that can advise the user such as, for example, a physician, technician or patient, regarding how to properly administer the composition(s) as prophylactic, therapeutic, or ameliorative treatment of the disease of concern. In some embodiments, instructions can indicate or suggest a dosing regimen that includes, but is not limited to, actual doses and monitoring procedures.

In some embodiments, the instructions can include informational material indicating that the administering of the compositions can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. In certain embodiments the informational material(s) may indicate that anaphylaxis can be fatal and may occur when any foreign protein is introduced into the body. In certain embodiments the informational material may indicate that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

While the instructional materials typically comprise written or printed materials they are not limited to such. Any medium capable of storing such instructions and communicating them to an end user is contemplated herein. Such media include, but are not limited to electronic storage media (e.g., magnetic discs, tapes, cartridges, chips), optical media (e.g., CD ROM), and the like. Such media may include addresses to internet sites that provide such instructional materials.

In some embodiments, the kits can comprise one or more packaging materials such as, for example, a box, bottle, tube, vial, container, sprayer, insufflator, intravenous (I.V.) bag, envelope, and the like; and at least one unit dosage form of an agent comprising active agent(s) described herein and a packaging material. In some embodiments, the kits also include instructions for using the composition as prophylactic, therapeutic, or ameliorative treatment for the disease of concern.

In some embodiments, the articles of manufacture can comprise one or more packaging materials such as, for example, a box, bottle, tube, vial, container, sprayer, insufflator, intravenous (I.V.) bag, envelope, and the like; and a first composition comprising at least one unit dosage form of an agent comprising one or more of alaproclate and/or other alaproclate “related” agents described herein, or a tautomer(s) or stereoisomer(s) thereof, or pharmaceutically acceptable salts or solvates of said alaproclate and other alaproclate “related” agents, said stereoisomer(s), or said tautomer(s), or analogues, derivatives, or prodrugs thereof within the packaging material, along with a second composition comprising a second agent such as, for example, an agent used in the treatment and/or prophylaxis of Alzheimer's disease (e.g., as described herein), or any prodrugs, codrugs, metabolites, analogs, homologues, congeners, derivatives, salts and combinations thereof. In some embodiments, the articles of manufacture may also include instructions for using the composition as a prophylactic, therapeutic, or ameliorative treatment for the disease of concern.

### EXAMPLES

The following examples are offered to illustrate, but not to limit the claimed invention.

### Example 1

### Identification of the Link Between ApoE4 and Sirtuin Expression

This example takes a highly innovative approach to therapeutic development, based on a new model for Alzheimer's disease (AD). Our studies link for the first time the major risk factor for Alzheimer's disease—ApoE4—with the major longevity determinants, the Sirtuins. Specifically, we show that the levels of SirT1 are significantly decreased in the presence of ApoE4 and there is increased production of pro-AD fragments such as sAPPβ (from which N-APP is derived), Aβ, Jcasp, and C31. In contrast, the trophic, anti-AD fragment sAPPα is significantly decreased.

This discovery provides a pathway to screen for modulators that would reverse the mechanisms underlying ApoE4 effects. Such modulators are useful, inter alia, as mechanistic probes and for development of ApoE4-targeted therapeutics.

The current results demonstrate the differential effects of ApoE4 versus ApoE3, supporting the finding that ApoE4 alters cellular APP processing, increasing the amyloidogenic/pro-AD processing of APP. Interestingly, in initial in vitro screens to identify compounds that reverse ApoE4 effects a number of “hits” have been revealed that target this new link, with the most promising one being the molecule alaproclate (“A03”), a known anti-depressant. Alaproclate increases both SirT1 and sAPPα levels in the presence of ApoE4 (see results). Thus this research has identified the first potential candidate therapeutic candidate based on this approach.

The agents and methods described herein are believed to be the first to target the ApoE4-induced decrease in SirT1 and sAPPα levels in the brain. The molecules identified herein address both the mechanistic and therapeutic aspects of targeting this risk factor. Such molecules additionally provide further understanding of multiple factors and pathways within the network around ApoE4 leading to the imbalance that underlies AD. This provides a novel and different therapeutic paradigm in AD. Given the current clinical landscape, it is likely that therapeutics targeting Aβ or tau-p alone will not address all of the pathogenic events in the disease. Targeting the ApoE4 risk factor and underlying mechanisms as described herein should lead to an effective treatment that could be used by itself or in combination current treatments in development for AD.

### Initial Studies

The following studies provide described research relevant to the development of small molecule SirT1 enhancers in AD. Experiments were performed on both cultured neural cells and in brain samples taken from patients with AD. Our research began with analysis of binding of ApoE to the ectodomain of APP and this ultimately led to the identification of mediators affected by ApoE4.

ApoE4 Binds to the Ectodomain of APP

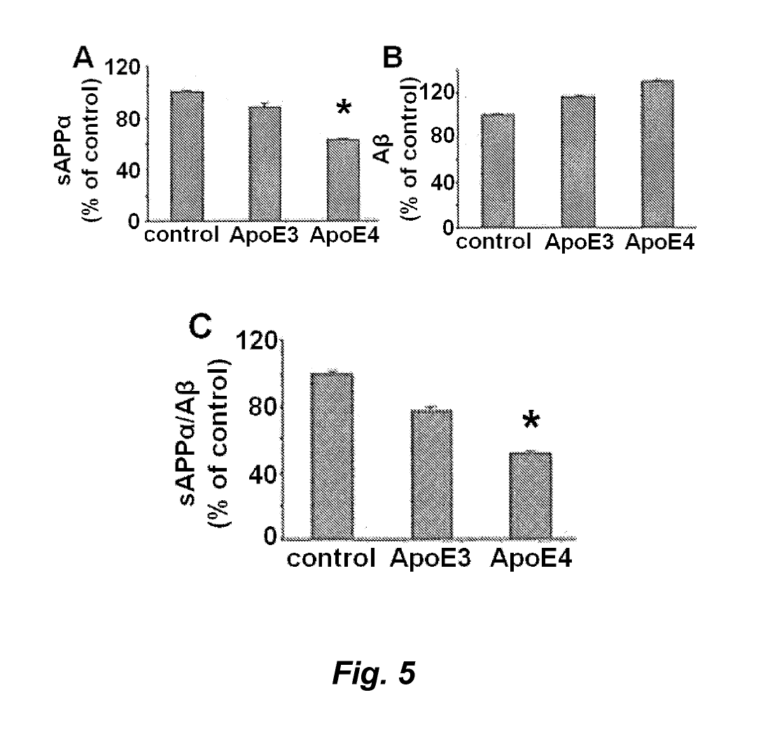

We confirmed the interaction of ApoE and APP by surface plasmon resonance (SPR) to measure the binding of recombinant ApoE isoforms with recombinant protein fragments of the ectodomain of APP, as well as the full ectodomain of APP695 (eAPP19-624) at a pH characteristic of intracellular compartments (FIG. 4, panels A and B). Analysis of the recombinant ApoE with a calibrated Superdex S-200 size-exclusion chromatography column gave the expected molecular weight of the ApoE tetramers for both ApoE3 and ApoE4, indicating that the amounts of lipid retained were small in comparison with the mass of the protein. The E2 domain of APP and the Aβ-cognate region (trx-eAPP290-624) gave an effective binding K_{D} of 80 nM for ApoE4.

Co-immunoprecipitation (Co-IP) experiments using cell extracts suggested that ApoE3 and ApoE4 associate with full length APP and/or sAPPα (from cell media) in both A172 human glioblastoma and H4 neuroglioma cell lines. In these experiments, IP with either an N-terminal (FIG. 4, panel C) or C-terminal (FIG. 4, panel D) anti-APP antibodies also precipitates ApoE3/E4. As A172 cells (4, panel C) express adequate levels of APP, they were transfected with ApoE isoforms only; the H4 cells (4, panel D) were transfected with both APP and ApoE. IP of the unrelated receptor protein TrkA from A172 cell lysates post-co-transfection with a human TrkA construct and ApoE isoforms did not reveal any association with E3 or E4, indicating the specificity of the APP-ApoE interaction. Although the lipidation status of ApoE affects its structure (Hatters et al. 92009) J. Mol. Biol. 386(1): 261-271; Hatters et al. (2005) J. Biol. Chem. 280(40): 34288-34295; Ye et al. Proc. Natl. Acad. Sci. USA, 102(51): 18700-18705) it is not yet clear whether the risk associated with ApoE4 is related solely to lipidated ApoE4, to poorly lipidated ApoE4, to unlipidated ApoE4, or to a combination of these forms of ApoE4. Therefore, by transfecting and allowing cellular lipidation to occur naturally, we can assess both the unlipidated and the lipidated forms with respect to their effects on APP binding and processing.

ApoE4 Transfection Reduces sAPPα Levels and Increases Aβ

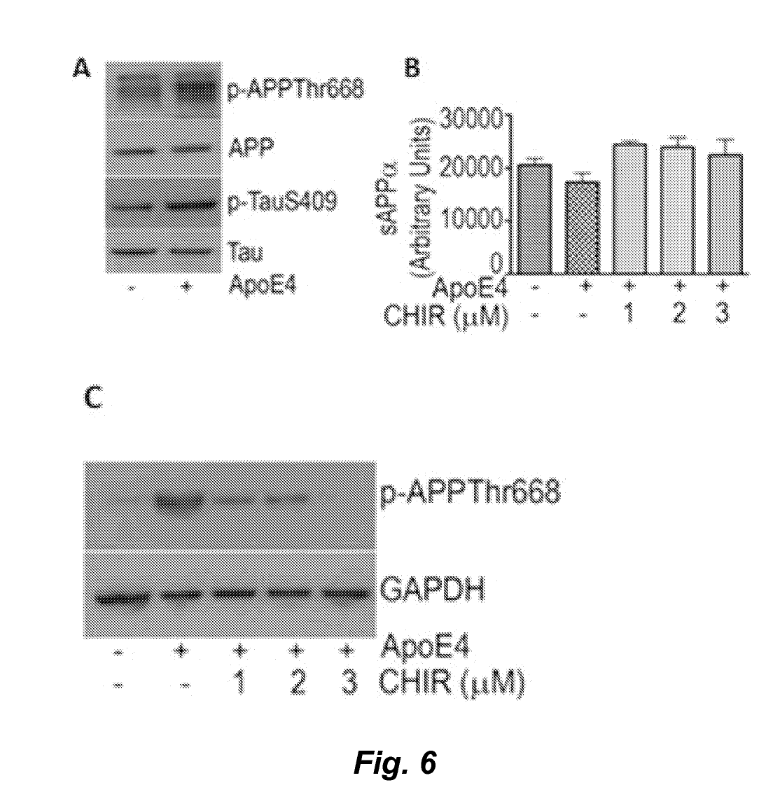

To understand the cellular consequences of ApoE-APP interactions further, we assessed the levels of sAPPα and Aβ in different cell lines. Expression of ApoE4, but not ApoE3, significantly decreased sAPPα secretion and reduced sAPPα/Aβ1-42 ratios in A172 human glioblastoma cells (FIG. 5) similar results were seen in H4 human neuroglioma cells. Our studies involved transfecting cells with either ApoE4 or. ApoE3 expression constructs for comparison and all experiments were performed in several different cell lines, some of which required transient transfection of ApoE isoforms alone (A172) and some that required transfections of amyloid precursor protein (APP) in addition to ApoE isoforms (HN33, HEK-293T, SHSY5Y, and H4 human neuroglioma). Thus far, our results demonstrate the following: (a) All of the cells that we tested expressed one or more of the receptors or specific ER chaperone proteins involved in the uptake of ApoE, such as the low-density lipoprotein (LDL) receptor (˜110 kD), LDL receptor-like protein (LRP; ˜85 kD), or receptor-associated protein (RAP, ˜24 kD); (b) Co-IP experiments suggest that ApoE3 and E4 associate with sAPPα (secreted in media) and APP (cell extracts) from several cell transfected with ApoE isoforms; (c) ApoE4, but not ApoE3, significantly reduces sAPPα and sAPPα/Aβ ratios. In addition, we looked at ApoE4Δ—the carboxyl-terminal truncated form of ApoE4—effects as it is known to be neurotoxic and triggers cell death (Harris et al. Proc. Natl. Acad. Sci. USA, 100(19): 10966-10971). A significant ApoE4 or ApoE4Δ isoform-dependent inhibition of sAPPα secretion and reduction in the α/β ratio was observed in neural cell lines as well (Theendakara et al. (2013) Proc. Natl. Acad. Sci. USA, 10(45): 18303-18308).

ApoE4 Transfection Increases p-Tau and p-APP

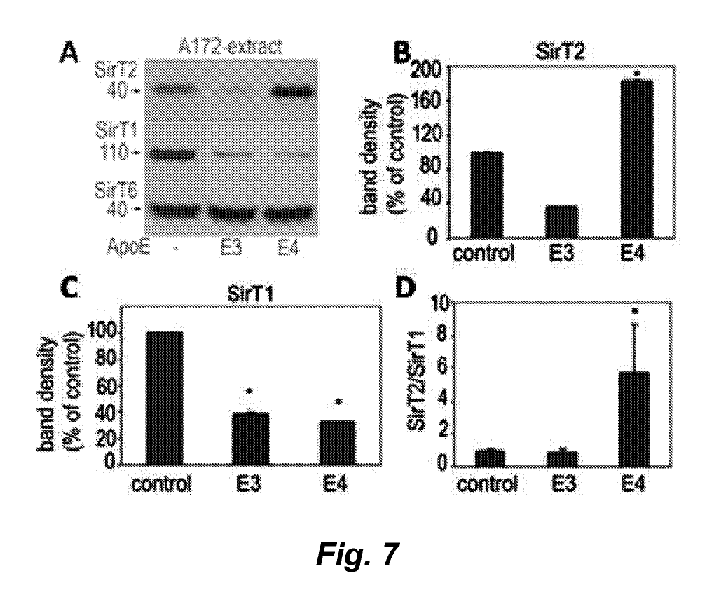

Transfection of ApoE4 increases APP-Thr668 phosphorylation and tau phosphorylation in cells (FIG. 6, panel A). As ApoE4 is thought to activate GSK-3β more than other isoforms, CHIR99021, a GSK-3β inhibitor, was used to treat cells transfected with ApoE4, and reversed the reduction in sAPPα (FIG. 6, panel B), as well as APP phosphorylation (FIG. 6, panel C). Similarly, the cyclin-dependent kinase5 (CDK5) inhibitor PHA793887 also attenuated p-APP and p-tau.

ApoE4 Transfection Reduces SirT1 Levels and Inverts the SirT1/SirT2 Ratio (“Sirtuinversion”)

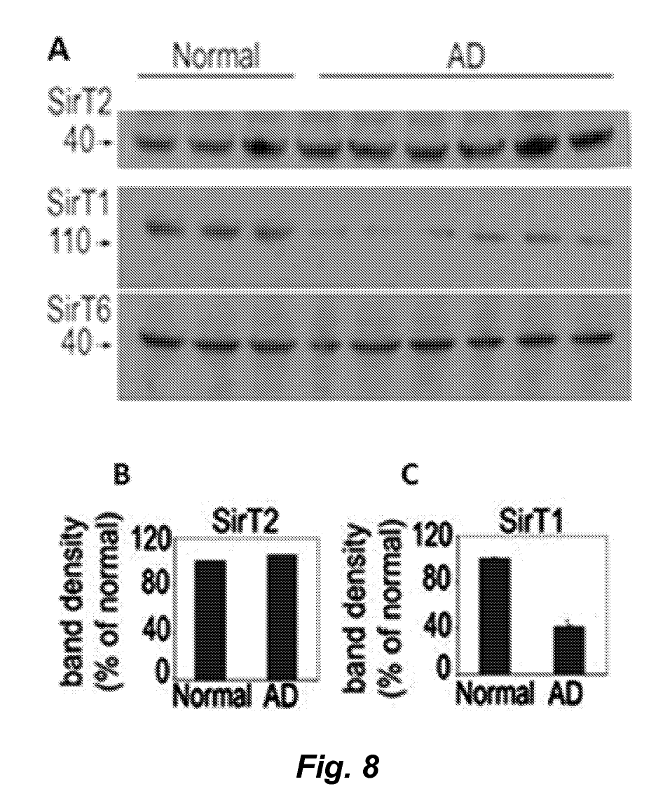

Our studies reveal that ApoE4 triggers a reduction in sAPPα levels is by inhibiting the proteolysis of APP at the α-site and associated with this is a reduction in transcription of SirT1. SirT1 belongs to the Sirtuin family of NAD-dependent protein deacetylases. SirT1 has previously been shown to suppress AD-related biochemical events in cells, primary neurons, and in AD mouse models by directly activating transcription of ADAM10, thus increasing the levels of neuroprotective sAPPα (Donmez et al. (2010) Cell, 142(2): 320-332; Donmez et al. (2013) Curr. Drug Targets, 14(6): 644-647). To investigate what role ApoE has on SirT1 level, we transfected A172 cells with ApoE3 and ApoE4 and performed WBs for SirT1, 2 and 6. ApoE4, but not ApoE3, increased SirT2, both decreased SirT1, and neither affected SirT6 protein levels (FIG. 7, panel A). We then looked at gene expression, performing quantitative real-time PCR in A172 cells transfected with ApoE3 or ApoE4, and saw similar results for SirT2 and SirT1 (FIG. 7, panels B and C, respectively). As a result the ratio of SirT2 to SirT1 was significantly increased in the presence of ApoE4 (FIG. 7, panel D).

Sirt1 Levels are Decreased in AD Brain

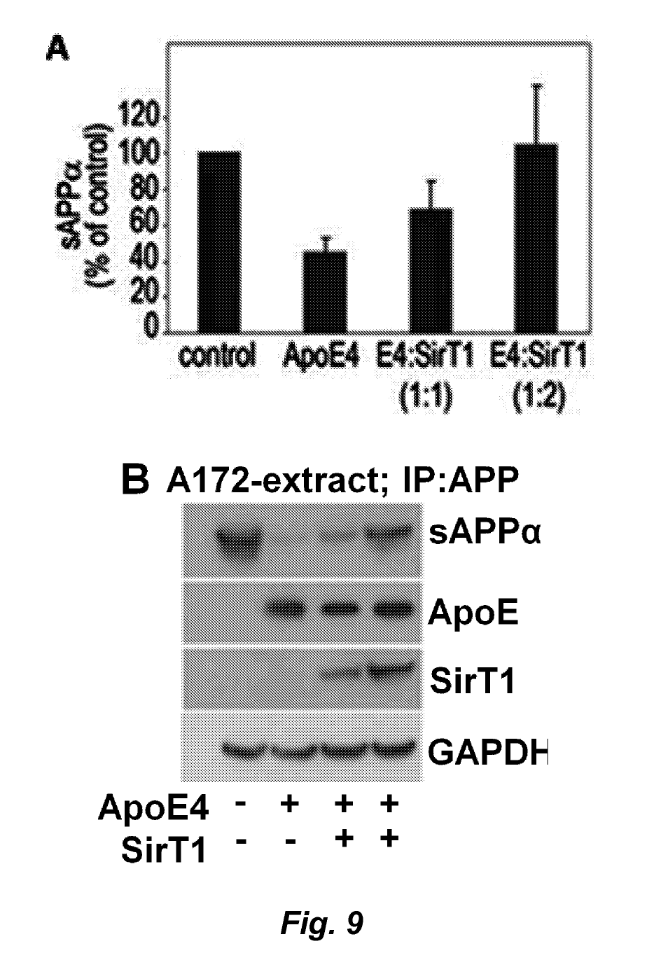

We evaluated the levels of SirT1, SirT2, and SirT6 in postmortem human brain tissue. All patients, with the exception of one normal control and one AD individual, were heterozygous for the ApoE alleles and all had one ApoE4 allele. SirT2 levels were slightly greater in AD samples compared to control (FIG. 8, panels A, B), SirT1 was greatly decreased in all AD samples (FIG. 8, panels A, C), and SirT6 was unchanged (FIG. 8, panel A). Tau and APP phosphorylation was increased in all AD samples with the exception of the one E3/E3 individual.

Overexpression of SirT1 Provides Target Validation.

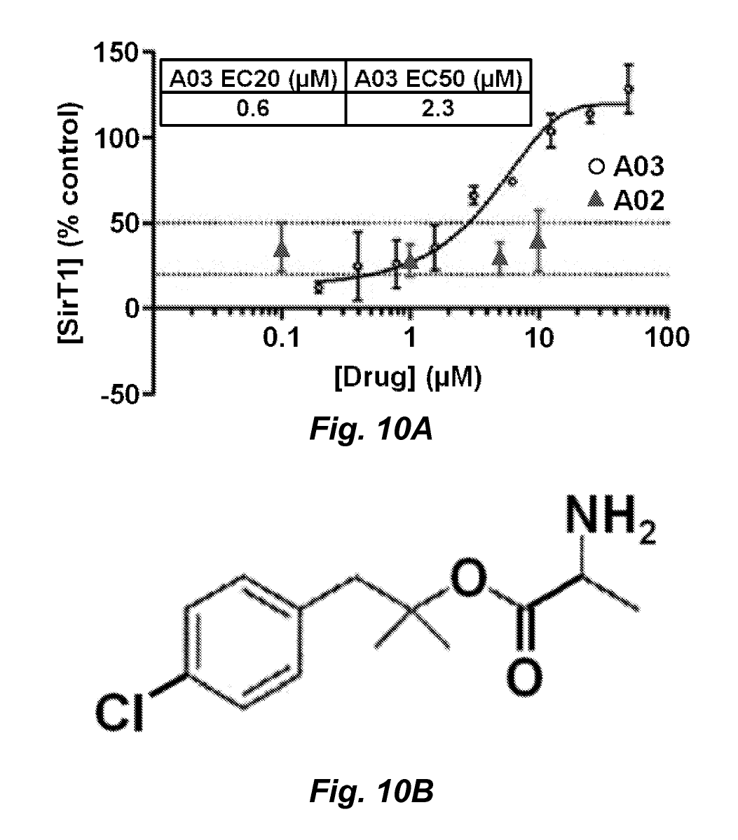

Because the ApoE4-mediated reduction in SirT1 levels may in turn result in reduced sAPPα levels, it was important to determine whether the restoration of SirT1 in the presence of ApoE4 reverses the reduction in sAPPα. Following transfection of A172 cells with ApoE4 and SirT1 (1:1 and 1:2, respectively), cell culture media were collected, and the levels of sAPPα were assessed. Overexpression of SirT1 reversed the ApoE4-mediated reduction in sAPPα secretion and restored it to normal levels (FIG. 9, panel A). Additionally, the cell extracts were immunoprecipitated with the N-terminal anti-APP antibody followed by SDS/PAGE and WB to detect sAPPα (FIG. 9, panel B). Thus, overexpression of SirT1 clearly reversed the ApoE4-mediated decrease in sAPPα expression. It is important to note that SirT1 activity is controlled post-translationally through phosphorylation of specific serines such as Ser47 (Sasaki et al. 92008) PLoS One3(12): e4020), thus alterations in phosphorylation may affect SirT1 effects.

Screening Hit that Increases SirT1 in an ApoE4-Transfected Cell Line

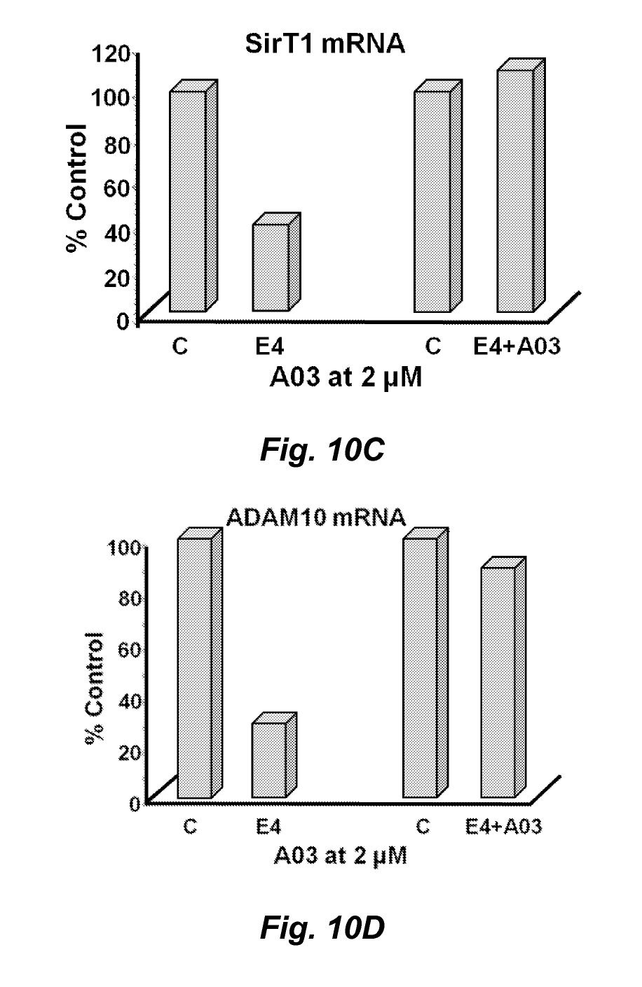

Initial screening of a small 1000-compound set yielded two primary hits, alaproclate (A03) and clonidine (A02). Only A03 gave a dose-response curve with SirT1 levels increasing with dose (FIG. 10A). In contrast, the response to A02 was flat. For A03, the EC20 for SirT1 enhancement was calculated at 0.2 uM while EC50 for SirT1 enhancement was about 2 uM.

A03 (FIG. 10 B) was originally developed as a serotonin reuptake inhibitor, but did not reach the market due to observed liver toxicity in rodents at high doses. In our initial screen there were other serotonin reuptake inhibitors such as Prozac that had no affect on SirT1 levels, suggesting that this is a unique property of A03 and independent of its serotonin reuptake inhibition property. Initial Phase1 human trials up to 200 mgs bid showed no observed adverse events and some improvements in global scores (Bergman et al. 91983) Psychopharmacology (Berl, 80(3): 279-283; Dehlin et al. (1985) Acta Psychiatr. Scand. 71(2): 190-196). A03 has excellent brain penetration brain/plasma (B/P ˜10). In our studies, A03 was seen to normalize the mRNA levels for SirT1 in A172 cells transfected with ApoE4 (FIG. 10C). Similarly a normalization of ADAM10 transcription was seen in these cells upon treatment with A03 at 2 uM (FIG. 10D). These two effects of A03 on A172 cells could occur serially, with the SirT1 increase first followed by increased ADAM10 and needs further study. While the complete sequence of events initiated by ApoE4 remains to be defined, it is clear that ApoE4 exerts isoform-specific effects on SirT1 and ADAM10 transcription, thus mediating APP processing favoring the amyloidogenic route, and also leads to APP and tau phosphorylation and SirT1 reduction; this combination may be a critical determinant of the ApoE4-associated risk for Alzheimer's disease.

### Conclusions

The preliminary data presented herein show that ApoE4 transfection results decrease sAPPα, increased Aβ and increased p-tau. We further demonstrate that the key mediator SirT1 is significantly decreased by ApoE4, but not ApoE3, transfection. This finding was confirmed in human brain tissue from patients with the ApoE ε4 allele. Target validation for SirT1 as a key mediator linked to ApoE4 is provided by overexpression studies where increased gene dose of SirT1 led to increased sAPPα levels. In addition we have identified in our initial screening a promising candidate that can pharmacological reverse the effects of ApoE4 on SirT1 levels and ADAM10. Taken together our data provide strong support for brain permeable enhancers of SirT1 as a therapeutic strategy for discovery of ApoE4-targeted therapeutics.

### Example 2

### Optimization and Validation of Lead Compounds

### Identify Validated ‘Hits’ Through Screening as Enhancers of SirT1 in ApoE4 Models.

Chemical libraries are screened using the primary AlphaLISA assay that we have developed to obtain hits that produce good dose-response activation of SirT1. The screening is done on all or a subset of the 200,000 compounds in the compound library (e.g., the UCLA compound library).

Initial Results.

As shown in FIG. 10A we can identify and obtain dose response-curves using our AlphaLISA assay. Our initial screening was done on a small library of about 1000 compounds. The assay is translatable to an HTS format.

Primary Screening Assay.

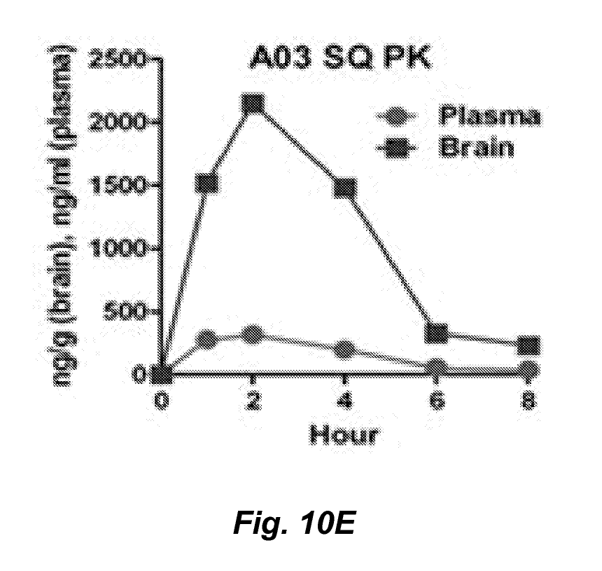

We have developed a primary screening assay that is HTS formatable; the assay would be done on N2a cells (mouse neuroblastoma cells) that have been stably transfected with ApoE3 and ApoE4. The cells are treated with drugs and the SirT1 levels are measured using the AlphaLISA assay that we have developed, e.g., as shown in FIG. 11. The illustrated AlphaLISA uses an antibody (Ab, E104, Abcam) that binds the C-terminus of SirT1 and is on the acceptor bead. The N-terminal Ab (D1D7, Abcam) is biotinylated and binds to the donor bead. For the primary assay the Neuro-2a (N2a) cells stably expressing similar levels of hApoE3 or hApoE4 are used. The primary assay can be done in triplicate. The N2a cells have reasonably good endogenous levels of APP thus avoiding the necessity of APP transfection. In general, for the assays N2a cells stably transfected with hApoE3 (E3) or hApoE4 (E4) are plated, e.g., at 10 cells/well in 384-well plates in 104 of growth medium and allowed to attached for 3 h. The E4 cells are treated with compounds overnight, E3 cells are controls. The following day, growth medium is removed and cells are lysed with AlphaLysis buffer complemented with protease and phosphatase inhibitors.

Then, the AlphaLISA assay is run and molecules that can increase or reverse the SirT1 levels in ApoE4 cells are identified. One embodiment of the AlphaLISA assay is shown in FIG. 11. A preliminary estimate, based on the initial screening which yielded two reproducible “hits” from a set of 1000 compounds, which is a 0.2% hit-rate. As shown in FIG. 10, we can identify and obtain dose response-curves using our AlphaLISA assay. Our initial screening was done on a small library of about 1000 compounds. The assay is translatable to a HTS format.

### Optimization of the Primary HTS Assay.

In various embodiments the screening strategy can optimized for HTS by miniaturization of the assay, linearity, CVs and Z-values. In one illustrative embodiment, the compound libraries consists of over 200,000 molecules that are split into 4 segments: pharmacological validation and repurposing libraries (Biomol, Prestwick and Microsource spectrum and NIH clinical collection), targeted libraries, lead-like libraries, diverse libraries (UCLA) and diverse sets of smart libraries. All compounds are at least 90% pure, typically better. On average, we find 95% of the hit compounds can be resupplied as powder for follow-up testing. With the exception of our diverse library which was a pre-plated set, all of our sets are custom sets and are not likely to be found in another screening facility. We have applied extensive filtering against liabilities such as reactive groups, aggregators, etc. (40) (additional description is available in the resource section).

Secondary in-Cell ELISA to Determine SirT1 vs SirT2 Selectivity.

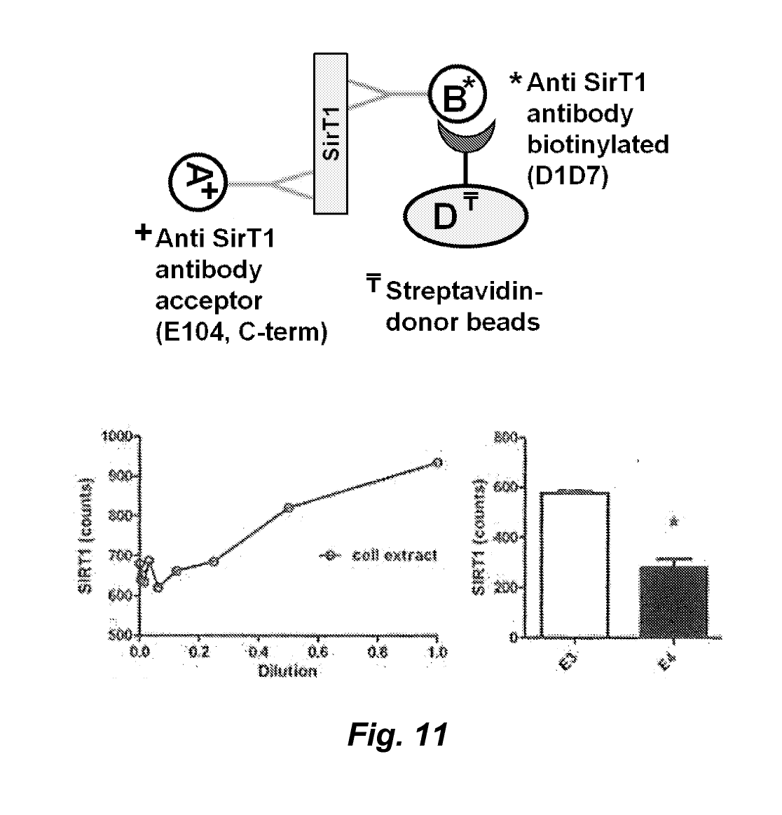

From the primary screen ‘hits’ that give a dose-response are evaluated in the secondary assay to determine selectivity in enhancing neuroprotective SirT1 while not significantly modulating neurotoxic SirT2. An in-cell ELISA approach can be used, e.g., as shown in FIG. 12. Commercial antibodies for SirT1 and SirT2 can be used and labeled with, e.g., AlexaFluor 488 or AlexaFluor 555.

Tertiary SirT1, sAPPα, sAPPβ, Aβ and p-Tau.

sAPPα or sAPPβ secreted into the cellular media are determined with sAPPα or sAPPβ AlphaLISA immunoassay kits (PerkinElmer) according to the manufacturer's protocol with some modifications. The standards, blanks, and media are diluted with the buffer provided in the kit and added to the plate. During the first incubation step, the analyte is captured either by an antibody recognizing the α-secretase cleavage site at sAPPα C-terminus (clone 2B3) or the sAPPβ C-terminus, and then by a second biotin-labeled antibody specific to the N-terminal common domain of sAPP. In the second incubation step, the biotinylated anti-analyte antibody is bound to the streptavidin-coated donor beads. At the end of this reaction, the plates are read on an EnSpire Alpha 2390 multilabel plate reader equipped with the AlphaScreen module. The Aβ1-42 or 1-40 are determined from media and/or cells using Life Science's sandwich ELISA kit. The levels of Aβ are quantified from a standard curve and normalized to total cellular protein (Spilman et al. (2014) Brain Res. 1551: 25-44; Theendakara et al. (2013) Proc. Natl. Acad. Sci. USA, 10(45): 18303-18308), Tau and p-tau are determined from cell extracts by a solid phase double antibody sandwich ELISA technology designed for quantitative determination of phospho-tau (Antibodies-Online). APP and p-APP are determined by the DuoSet IC ELISA (R&D Systems) to measure phosphorylated APP in cell lysates. An immobilized capture antibody specific for APP binds both phosphorylated and unphosphorylated APP. After unbound material is washed away, a biotinylated detection antibody is used to detect either APP or phosphor-APP utilizing a standard HRP format. Using these assay procedures we notice a significant increase in p-tau and p-APP levels in ApoE4-transfected cells. It can be determined whether the primary “hits” reverse the ApoE4-mediated APP-Thr phosphorylation and tau-phosphorylation.

### Quantitative Real-Time PCR.

Total RNA from transfected and drug-treated cells is isolated using the High Pure RNA isolation kit (Roche) and 1 μg of RNA is reverse-transcribed. To analyze the mRNA levels of SirT1, real time PCR can be performed on first-strand cDNAs as described (Theendakara et al. (2013) Proc. Natl. Acad. Sci. USA, 10(45): 18303-18308). The real-time PCR can be performed in Light Cycler 480-384-multiwell plates (Roche). Primers for SirT1 were designed using the Roche universal probe library system and the primers were synthesized by Integrated DNA Technology. Real-time PCR can be performed in SYBR Green master mix with the corresponding primer sets. The melting curves of PCR products are monitored to ensure that a single melting curve is obtained. For analysis of the real-time PCR data, δCt values of samples are normalized to values obtained for GAPDH, which is assayed simultaneously. Relative quantification using the DeltaDelta Ct method can be adopted to calculate the relative quantity of SirT1 levels.

### Other Strategies

If the primary screening assay yields more than the desired number of hits the selection criteria can be adjusted to keep the hit rate at 0.2% and validated hits can be obtained after the secondary and tertiary screening. Higher numbers can be triaged based on adjustment to the selection criteria and “drugable” structures. The In-cell ELISA for selectivity can optionally be replaced with a AlphaLisa assay.

### Example 3

### Synthesis of Analogs 1-24 for Increases in SirT1 Levels in ApoE4 Cells

### Boc Protected Esters; General Procedure (Analogs 1-14; 16-21 & 23)

In a round bottom flask equipped with a stir bar, the tertiary alcohol (1.5 mmol) was dissolved with an appropriate amount of dichloromethane to give 2M concentration of the alcohol. The solution was added 4-dimethylaminopyridine (DMAP, 1.5 mmol) and the Boc-protected amino acid (3 mmol). The solution was allowed to stir for 10 min on ice before adding N-(3-dimethylaminopropyl)-N′-ethylcarbodiimide hydrochloride (EDC HCl), 3 mmol). The reaction mixture was allowed to warm to room temperature and stirred overnight. The solution was concentrated under vacuum, and the product was isolated by flash chromatography (EtOAc/Hexanes=1:6).

### Boc-Protected Amides; General Procedure (Analogs 15, 22 &24)

In a round bottom flask equipped with a stir bar, the tertiary amine (1.5 mmol) was dissolved with an appropriate amount of dichloromethane to give 2M concentration of the alcohol. The solution was added 4-dimethylaminopyridine (DMAP, 0.2 mmol) and the Boc-protected amino acid (3 mmol). The solution was allowed to stir for 10 min on ice before adding N-(3-dimethylaminopropyl)-N-ethylcarbodiimide hydrochloride (EDC HCl), 3 mmol). The reaction mixture was allowed to stir on ice for 1 hr, and then warmed to room temperature to stir for an additional 2 hr. The solution was concentrated under vacuum, and the product was isolated by flash chromatography (EtOAc/Hexanes=1:6).

### Boc Deprotection and Generation of Compounds; General Procedure (Table 5, Analogs 1-24)

In a round bottom flask equipped with a stir bar, the N-Boc protected esters and amides (0.5 mmol) were cooled to 0° C. and was added 1.25 mL of 4M HCl in dioxane and allowed to stir on ice for 1 hr. The solution was allowed to stir for an additional 1 hr at room temperature. The solution was concentrated under vacuum, and was added ether or hexanes. The hydrochloride salt was allowed to precipitate, and the product was filtered and dried. Some of the hydrochloride salt either dissolved the product or resulted in a waxy form with ether solvent. In these cases, the ether was evaporated, and the product was added hexanes and sonicated for 10 min before decanting the hexane solvent, and dried under high vacuum.

TABLE 6
Illustrative analogs evaluated in the primary screening assay

| Compound | | | | Primary |
|---|---|---|---|---|
| # | Structure | MW | cLogP | Assay |
| 1 | 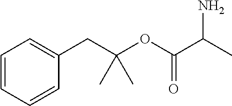 [SMILES: CC(N)C(=O)OC(C)(C)Cc1ccccc1] | 221 | 2.37 | II |
| 2 | 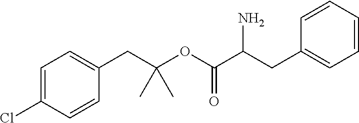 [SMILES: CC(C)(Cc1ccc(Cl)cc1)OC(=O)C(N)Cc1ccccc1] | 332 | 4. 5 | I |
| 3 | 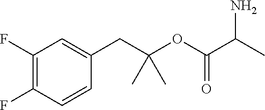 [SMILES: CC(N)C(=O)OC(C)(C)Cc1ccc(F)c(F)c1] | 257 | 2.58 | III |
| 4 | 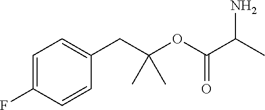 [SMILES: CC(N)C(=O)OC(C)(C)Cc1ccc(F)cc1] | 239 | 2.87 | II |
| 5 | 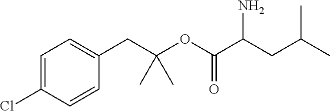 [SMILES: CC(C)CC(N)C(=O)OC(C)(C)Cc1ccc(Cl)cc1] | 298 | 4.54 | I |
| 6 | 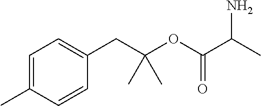 [SMILES: Cc1ccc(CC(C)(C)OC(=O)C(C)N)cc1] | 235 | 2.87 | II |
| 7 | 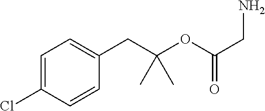 [SMILES: CC(C)(Cc1ccc(Cl)cc1)OC(=O)CN] | 242 | 2.77 | II |
| 8 | 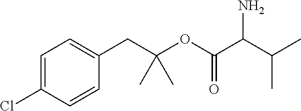 [SMILES: CC(C)C(N)C(=O)OC(C)(C)Cc1ccc(Cl)cc1] | 284 | 4.01 | II |
| 9 | 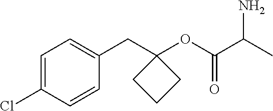 [SMILES: CC(N)C(=O)OC1(Cc2ccc(Cl)cc2)CCC1] | 268 | 3.27 | I |
| 10 | 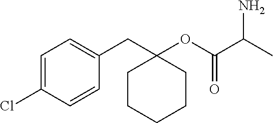 [SMILES: CC(N)C(=O)OC1(Cc2ccc(Cl)cc2)CCCCC1] | 296 | 4.39 | I |
| 11 | 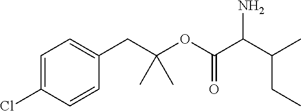 [SMILES: CCC(C)C(N)C(=O)OC(C)(C)Cc1ccc(Cl)cc1] | 298 | 3.95 | II |
| 12 | 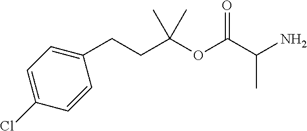 [SMILES: CC(N)C(=O)OC(C)(C)CCc1ccc(Cl)cc1] | 270 | 3.19 | II |
| 13 | 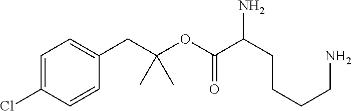 [SMILES: CC(C)(Cc1ccc(Cl)cc1)OC(=O)C(N)CCCCN] | 313 | 2.18 | II |
| 14 | 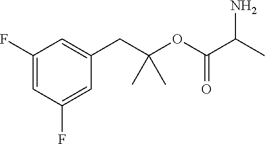 [SMILES: CC(N)C(=O)OC(C)(C)Cc1cc(F)cc(F)c1] | 257 | 2.25 | III |
| 15 | 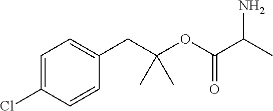 [SMILES: CC(N)C(=O)OC(C)(C)Cc1ccc(Cl)cc1] | 255 | 1.92 | III |
| 16 | 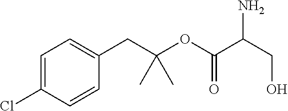 [SMILES: CC(C)(Cc1ccc(Cl)cc1)OC(=O)C(N)CO] | 272 | 1.70 | II |
| 17 | 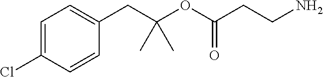 [SMILES: CC(C)(Cc1ccc(Cl)cc1)OC(=O)CCN] | 256 | 2.23 | I |
| 18 | 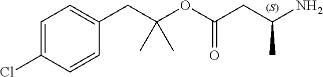 | 270 | 2.64 | I |
| 19 | 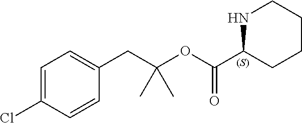 | 296 | 4.15 | II |
| 20 | 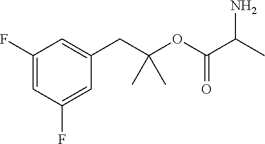 [SMILES: CC(N)C(=O)OC(C)(C)Cc1cc(F)cc(F)c1] | 243 | 1.85 | I |
| 21 | 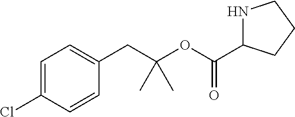 [SMILES: CC(C)(Cc1ccc(Cl)cc1)OC(=O)C1CCCN1] | 282 | 3.64 | II |
| 22 | 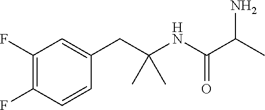 [SMILES: CC(N)C(=O)NC(C)(C)Cc1ccc(F)c(F)c1] | 256 | 1.49 | III |
| 23 | 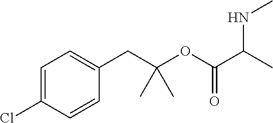 [SMILES: CNC(C)C(=O)OC(C)(C)Cc1ccc(Cl)cc1] | 270 | 3.58 | I |
| 24 | 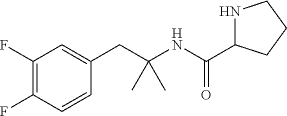 [SMILES: CC(C)(Cc1ccc(F)c(F)c1)NC(=O)C1CCCN1] | 282 | 2.46 | II |

In Table 6 activity I < 10%; activity II 10-30%; activity III > 30 SrT1 increase.

### Product Compounds (as HCl Salts):

Analog 1:

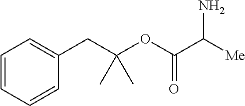 [SMILES: CC(N)C(=O)OC(C)(C)Cc1ccccc1]

2-methyl-1-phenylpropan-2-yl 2-aminopropanoate: ¹H NMR (400 MHz, MeOD) δ 7.27 (m, 5H), 3.95 (q, 1H, J=7.2 Hz), 3.12 (d, 2H, 2.9 Hz), 1.52 (s, 3H) 1.51 (s, 3H), 1.42 (d, 3H, J=7.2 Hz). ¹³C NMR (400 MHz, MeOD) δ 168.9, 136.5, 130.3, 127.7, 126.4, 85.3, 48.9, 45.8, 24.9, 24.5, 14.9.

Analog 2:

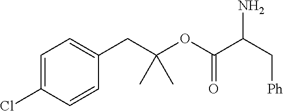 [SMILES: CC(C)(Cc1ccc(Cl)cc1)OC(=O)C(N)Cc1ccccc1]

1-(4-chlorophenyl)-2-methylpropan-2-yl 2-amino-3-phenylpropanoate: ¹H NMR (400 MHz, MeOD) δ 7.37-7.12 (m, 9H), 4.15 (t, 1H, J=7.0 Hz), 3.10 (d, 2H, J=7.0 Hz), 2.99 (d, 1H, J=13.7 Hz), 2.90 (d, 1H, J=13.7 Hz), 1.46 (s, 3H) 1.42 (s, 3H). ¹³C NMR (400 MHz, MeOD) δ 168.1, 135.2, 134.3, 131.9. 132.4, 129.1, 128.7, 127.8, 127.5, 85.2, 54.2, 45.6, 36.4, 24.4, 24.3.

Analog 3:

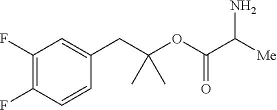 [SMILES: CC(N)C(=O)OC(C)(C)Cc1ccc(F)c(F)c1]

1-(3,4-difluorophenyl)-2-methylpropan-2-yl 2-aminopropanoate: ¹H NMR (500 MHz, MeOD) δ 7.18 (m, 2H), 7.02 (m, 1H), 3.97 (q, 1H, J=7.2 Hz), 3.10 (d, 2H, J=3.5 Hz), 1.52 (s, 3H) 1.51 (s, 3H), 1.42 (d, 3H, J=7.0 Hz). ¹³C NMR (500 MHz, MeOD) δ 169.0, 150.5 (dd, J=49.9, 12.6 Hz), 148.5 (dd, J=49.4, 12.6 Hz), 134.0 (dd, J=5.7, 4.0 Hz), 129.8 (dd, J=6.2, 3.5 Hz), 119.0 (d, J=17.1 Hz), 116.4 (d, J=17.2 Hz), 84.8, 48.6, 44.9, 24.7, 24.4, 14.9.

Analog 4:

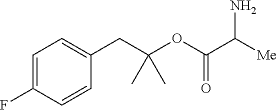 [SMILES: CC(N)C(=O)OC(C)(C)Cc1ccc(F)cc1]

1-(4-fluorophenyl)-2-methylpropan-2-yl 2-aminopropanoate: ¹H NMR (500 MHz, MeOD) δ 7.23 (m, 2H), 7.02 (m, 2H), 3.95 (q, 1H, J=7.2 Hz), 3.10 (d, 2H, J=2.3 Hz), 1.52 (s, 3H) 1.49 (s, 3H), 1.42 (d, 3H, J=7.2 Hz). ¹³C NMR (500 MHz, MeOD) δ 169.0, 161.9 (d, J=243.6 Hz), 132.5 (d, J=3.3 Hz), 131.9 (d, J=7.9 Hz), 114.3 (d, J=21.4 Hz), 85.1, 48.9, 44.9, 24.7, 24.4, 14.9.

Analog 5:

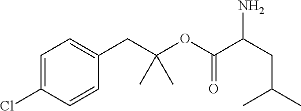 [SMILES: CC(C)CC(N)C(=O)OC(C)(C)Cc1ccc(Cl)cc1]

1-(4-chlorophenyl)-2-methylpropan-2-yl 2-amino-4-methylpentanoate: ¹H NMR (500 MHz, MeOD) δ 7.30 (d, 2H, J=8.4 Hz), 7.22 (d, 2H, J=8.4 Hz), 3.86 (dd, 1H, J=7.6, 6.1 Hz), 3.12 (d, 1H, J=13.7 Hz), 3.07 (d, 1H, J=13.7 Hz), 1.65-1.50 (m, 9H), 0.92 (m, 6H). ¹³C NMR (500 MHz, MeOD) δ 169.0, 135.3, 132.5, 131.9, 127.8, 85.1, 51.4, 45.2, 39.4, 24.9, 24.4, 24.1, 21.2, 20.7.

Analog 6:

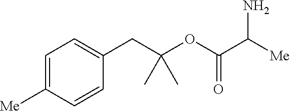 [SMILES: Cc1ccc(CC(C)(C)OC(=O)C(C)N)cc1]

2-methyl-1-p-tolylpropan-2-yl 2-aminopropanoate: ¹H NMR (500 MHz, MeOD) δ 7.10 (m, 4H), 3.94 (q, 1H, J=7.2 Hz), 3.09 (d, 1H, J=13.7 Hz), 3.06 (d, 1H, J=13.7 Hz), 2.30 (s, 3H), 1.51 (s, 3H), 1.50 (s, 3H), 1.43 (d, 3H, J=7.3 Hz). ¹³C NMR (500 MHz, MeOD) δ 168.9, 136.1, 133.3, 130.2, 128.3, 85.4, 48.9, 45.3, 24.8, 24.5, 19.7, 14.9.

Analog 7:

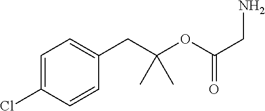 [SMILES: CC(C)(Cc1ccc(Cl)cc1)OC(=O)CN]

1-(4-chlorophenyl)-2-methylpropan-2-yl 2-aminoethanoate: ¹H NMR (500 MHz, MeOD) δ 7.30 (d, 2H, J=8.4 Hz), 7.21 (d, 2H, J=8.4 Hz), 3.71 (s, 2H), 3.11 (s, 2H), 1.51 (s, 6H). ¹³C NMR (500 MHz, MeOD) δ 166.4, 135.4, 132.4, 131.8, 127.8, 85.0, 45.0, 40.2, 24.7.

Analog 8:

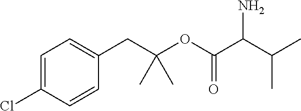 [SMILES: CC(C)C(N)C(=O)OC(C)(C)Cc1ccc(Cl)cc1]

1-(4-chlorophenyl)-2-methylpropan-2-yl 2-amino-3-methylbutanoate: ¹H NMR (500 MHz, MeOD) δ 7.31 (d, 2H, J=8.4 Hz), 7.24 (d, 2H, J=8.4 Hz), 3.80 (d, 1H, J=4.4 Hz), 3.16 (d, 1H, J=13.7 Hz), 3.03 (d, 1H, J=13.7 Hz), 2.24 (qd, 1H, J=7.0, 4.3 Hz), 1.57 (s, 3H), 1.49 (s, 3H) 1.04 (d, 3H, J=7.0 Hz), 1.00 (d, 3H, J=7.0 Hz). ¹³C NMR (500 MHz, MeOD) δ 168.0, 135.2, 132.4, 132.1, 127.8, 85.4, 58.3, 45.7, 29.5, 24.6, 24.4, 16.9, 16.7.

Analog 9:

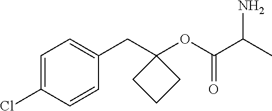 [SMILES: CC(N)C(=O)OC1(Cc2ccc(Cl)cc2)CCC1]

1-(4-chlorobenzyl)cyclobutyl 2-aminopropanoate: ¹H NMR (500 MHz, MeOD) δ 7.33 (d, 2H, J=8.4 Hz), 7.23 (d, 2H, J=8.4 Hz), 4.02 (q, 1H, J=7.2 Hz), 3.30 (m, 2H), 2.41 (m, 4H), 1.92 (m, 1H), 1.73 (m, 1H), 1.44 (d, 3H, J=7.2 Hz). ¹³C NMR (500 MHz, MeOD) δ 168.4, 135.2, 132.3, 131.1, 127.9, 84.3, 48.5, 39.7, 33.1, 33.0, 14.7, 12.9.

Analog 10:

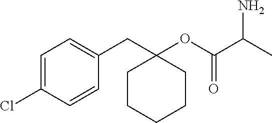 [SMILES: CC(N)C(=O)OC1(Cc2ccc(Cl)cc2)CCCCC1]

1-(4-chlorobenzyl)cyclohexyl 2-aminopropanoate: ¹H NMR (500 MHz, MeOD) δ 7.29 (d, 2H, J=8.0 Hz), 7.15 (d, 2H, J=8.0 Hz), 3.99 (q, 1H, J=7.1 Hz), 3.24 (m, 2H), 2.25 (m, 2H), 1.68-1.40 (m, 10H), 1.32 (m, 1H). ¹³C NMR (500 MHz, MeOD) δ 169.1, 134.8, 132.4, 131.7, 127.8, 86.8, 48.6, 42.2, 33.8, 33.7 24.8, 21.3, 15.1.

Analog 11:

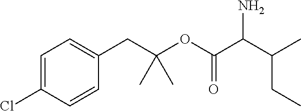 [SMILES: CCC(C)C(N)C(=O)OC(C)(C)Cc1ccc(Cl)cc1]

1-(4-chlorophenyl)-2-methylpropan-2-yl 2-amino-3-methylpentanoate: ¹H NMR (500 MHz, CDCl₃) δ 8.79 (bs, 3H), 7.26 (d, 2H, J=8.1 Hz), 7.13 (d, 2H, 8.1 Hz), 3.86 (m, 1H), 3.18-2.96 (m, 2H), 2.08 (m, 1H), 1.76-1.34 (m, 8H), 1.08-0.87 (m, 6H). ¹³C NMR (500 MHz, CDCl₃) δ 167.7 (diast. A), 167.3 (diast. B), 134.9, 132.7, 132.0, 128.3, 85.6 (diast. A), 85.8 (diast. B), 57.6 (diast. A), 57.8 (diast. B), 45.9 (diast. A), 45.8 (diast. B), 36.7 (diast. B), 36.4 (diast. A), 25.6, 25.5, 15.0, 14.4, 11.8 (diast. A), 11.7 (diast. B).

Analog 12:

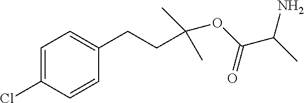 [SMILES: CC(N)C(=O)OC(C)(C)CCc1ccc(Cl)cc1]

4-(4-chlorophenyl)-2-methylbutan-2-yl 2-aminopropanoate: ¹H NMR (500 MHz, MeOD) δ 7.26 (d, 2H, J=8.4 Hz), 7.18 (d, 2H, J=8.4 Hz), 3.97 (q, 1H, J=7.2 Hz), 2.66 (m, 2H), 2.11 (m, 2H), 1.56 (s, 6H), 1.51 (d, 3H, J=7.3 Hz). ¹³C NMR (500 MHz, MeOD) δ 168.8, 140.5, 131.3, 129.6, 128.1, 85.4, 48.9, 42.0, 29.2, 24.8, 24.7, 15.0.

Analog 13:

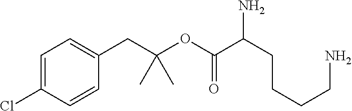 [SMILES: CC(C)(Cc1ccc(Cl)cc1)OC(=O)C(N)CCCCN]

1-(4-chlorophenyl)-2-methylpropan-2-yl 2,6-diaminohexanoate: ¹H NMR (500 MHz, MeOD) δ 7.32 (d, 2H, J=8.4 Hz), 7.23 (d, 2H, J=8.4 Hz), 3.92 (t, 1H, J=6.5 Hz), 3.11 (s, 2H), 2.87 (t, 2H, J=7.9 Hz), 1.83 (m, 2H), 1.64 (m, 2H), 1.54 (s, 3H), 1.51 (s, 3H), 1.39 (m, 2H). ¹³C NMR (500 MHz, MeOD) δ 168.4, 135.3, 132.4, 132.0, 127.9, 85.4, 52.8, 45.2, 38.8, 29.8, 26.7, 24.9, 24.4, 21.4.

Analog 14:

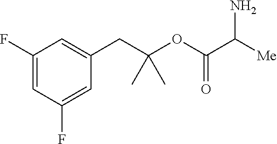 [SMILES: CC(N)C(=O)OC(C)(C)Cc1cc(F)cc(F)c1]

1-(3,5-difluorophenyl)-2-methylpropan-2-yl 2-aminopropanoate: ¹H NMR (500 MHz, CDCl₃) δ 8.78 (bs, 3H), 6.69 (m, 3H), 4.10 (q, 1H, J=7.1 Hz), 3.05 (m, 2H), 1.64 (d, 3H, J=7.2 Hz), 1.48 (s, 3H), 1.46 (s, 3H). ¹³C NMR (500 MHz, CDCl₃) δ 168.9, 163.6 (d, J=12.9), 161.7 (d, J=12.8), 140.2 (t, J=9.2 Hz), 113.4 (d, J=25.6 Hz), 102.3 (t, J=25.2 Hz), 84.9, 49.7, 46.3, 25.8, 25.5, 16.2.

Analog 15:

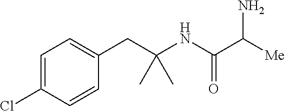 [SMILES: CC(N)C(=O)NC(C)(C)Cc1ccc(Cl)cc1]

2-amino-N-(1-(4-chlorophenyl)-2-methylpropan-2-yl)propanamide: ¹H NMR (500 MHz, MeOD) δ 7.26 (d, 2H, J=8.3 Hz), 7.13 (d, 2H, J=8.3 Hz), 3.78 (t, 1H, J=7.0 Hz), 3.13 (s, 1H, J=13.7 Hz), 2.98 (d, 1H, J=13.7 Hz), 1.41 (d, 3H, J=7.0 Hz), 1.32 (m, 3H), 1.28 (s, 3H). ¹³C NMR (500 MHz, MeOD) δ 168.8, 136.6, 132.0, 131.8, 127.6, 53.9, 49.0, 42.9, 25.9, 16.7.

Analog 16:

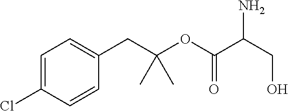 [SMILES: CC(C)(Cc1ccc(Cl)cc1)OC(=O)C(N)CO]

1-(4-chlorophenyl)-2-methylpropan-2-yl 2-amino-3-hydroxypropanoate: ¹H NMR (500 MHz, MeOD) δ 7.29 (d, 2H, J=8.2 Hz), 7.22 (d, 2H, J=8.1 Hz), 3.97 (m, 1H), 3.86 (m, 2H), 3.10 (m, 2H), 1.52 (s, 3H), 1.49 (s, 3H). ¹³C NMR (500 MHz, MeOD) δ 166.8, 135.3, 132.3, 131.9, 127.8, 85.2, 59.3, 55.1, 45.1, 24.7, 24.5.

Analog 17:

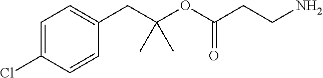 [SMILES: CC(C)(Cc1ccc(Cl)cc1)OC(=O)CCN]

1-(4-chlorophenyl)-2-methylpropan-2-yl 3-aminopropanoate: ¹H NMR (500 MHz, MeOD) δ 7.28 (d, 2H, J=8.5 Hz), 7.19 (d, 2H, J=8.5 Hz), 3.13 (t, 1H, J=6.6 Hz), 3.00 (s, 2H), 2.64 (t, 2H, J=6.6 Hz), 1.47 (s, 6H). ¹³C NMR (500 MHz, MeOD) δ 170.2, 135.7, 132.2, 131.8, 127.7, 83.2, 45.2, 34.9, 31.8, 24.7.

Analog 18:

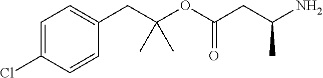 [SMILES: C[C@H](N)CC(=O)OC(C)(C)Cc1ccc(Cl)cc1]

(S)-1-(4-chlorophenyl)-2-methylpropan-2-yl 3-aminobutanoate: ¹H NMR (500 MHz, CDCl₃) δ 8.51 (bs, 3H), 7.27 (d, 2H, J=8.3 Hz), 7.08 (d, 2H, J=8.3 Hz), 3.69 (m, 1H), 3.00 (s, 2H), 2.72 (m, 2H), 1.50 (d, 3H, J=5.6 Hz), 1.44 (s, 3H), 1.43 (s, 3H). ¹³C NMR (500 MHz, CDCl₃) δ 170.6, 135.1, 132.7, 131.8, 128.4, 84.3, 45.9, 45.3, 38.6, 25.9, 25.8, 18.4.

Analog 19:

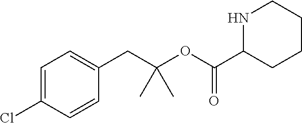 [SMILES: CC(C)(Cc1ccc(Cl)cc1)OC(=O)C1CCCCN1]

1-(4-chlorophenyl)-2-methylpropan-2-yl piperidine-2-carboxylate: ¹H NMR (500 MHz, CDCl₃) δ 10.25 (bs, 1H), 9.57 (bs, 1H), 7.27 (d, 2H, J=8.4 Hz), 7.14 (d, 2H, J=8.4 Hz), 3.83 (m, 1H), 3.58 (m, 1H), 3.07 (m, 3H), 2.14 (m, 1H), 2.02-1.85 (m, 3H), 1.68 (m, 1H), 1.56 (m, 1H), 1.49 (s, 3H), 1.45 (s, 3H). ¹³C NMR (500 MHz, CDCl₃) δ 167.4, 134.8, 132.8, 131.9, 128.4, 85.6, 56.5, 45.5, 43.3, 26.0, 25.9, 25.7, 21.7, 21.4.

Analog 20:

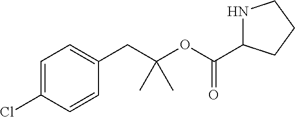 [SMILES: CC(C)(Cc1ccc(Cl)cc1)OC(=O)C1CCCN1]

1-(4-chlorophenyl)-2-methylpropan-2-yl pyrrolidine-2-carboxylate: ¹H NMR (500 MHz, MeOD) δ 7.30 (d, 2H, J=8.3 Hz), 7.21 (d, 2H, J=8.3 Hz), 4.29 (m, 1H), 3.36 (m, 2H), 3.09 (s, 2H), 2.34 (m, 1H), 2.07-1.90 (m, 3H), 1.53 (s, 3H), 1.50 (s, 3H). ¹³C NMR (500 MHz, MeOD) δ 168.0, 135.3, 132.5, 131.9, 127.8, 85.5, 59.8, 45.7, 45.3, 28.0, 24.7, 24.4, 23.1.

Analog 21:

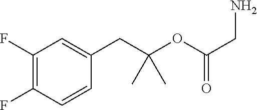 [SMILES: CC(C)(Cc1ccc(F)c(F)c1)OC(=O)CN]

1-(3,4-difluorophenyl)-2-methylpropan-2-yl 2-aminoethanoate: ¹H NMR (500 MHz, MeOD) δ 7.17 (m, 2H), 7.02 (m, 1H), 3.72 (s, 2H), 3.09 (s, 2H), 1.50 (s, 6H). ¹³C NMR (500 MHz, CDCl₃) δ 166.4, 150.5 (d, J=55.6, 12.7 Hz), 148.5 (dd, J=54.8, 12.6 Hz), 134.1 (dd, J=5.7, 4.1 Hz), 126.7 (d, J=3.4 Hz), 118.9 (d, J=17.0 Hz), 116.4 (d, J=17.2 Hz), 84.9, 44.9, 40.1, 24.6.

Analog 22:

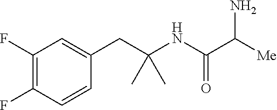 [SMILES: CC(N)C(=O)NC(C)(C)Cc1ccc(F)c(F)c1]

2-amino-N-(1-(3,4-difluorophenyl)-2-methylpropan-2-yl)propanamide: ¹H NMR (500 MHz, MeOD) δ 7.13 (m, 1H), 7.03 (m, 1H), 6.92 (m, 1H), 3.75 (q, 1H, J=7.0 Hz), 3.15 (d, 1H, J=13.3 Hz), 2.94 (d, 2H, J=13.3 Hz), 1.39 (d, 3H, J=7.0 Hz), 1.32 (s, 3H), 1.26 (s, 3H). ¹³C NMR (500 MHz, MeOD) δ 168.8, 150.3 (dd, J=69.9, 12.6 Hz), 148.4 (dd, J=69.3, 12.6 Hz), 135.3 (dd, J=5.7, 4.2 Hz), 126.6 (dd, J=6.1, 3.5 Hz), 118.7 (d, J=17.1 Hz), 116.2 (d, J=17.1 Hz), 53.9, 49.0, 42.7, 25.9, 16.6.

Analog 23:

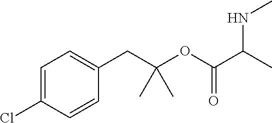 [SMILES: CNC(C)C(=O)OC(C)(C)Cc1ccc(Cl)cc1]

1-(4-chlorophenyl)-2-methylpropan-2-yl 2-(methylamino)propanoate: ¹H NMR (500 MHz, CDCl₃) δ 10.00 (bs, 1H), 9.69 (bs, 1H), 7.27 (m, 2H), 7.13 (d, 2H, J=7.6 Hz), 3.77 (m, 1H), 3.07 (m, 1H), 2.72 (s, 3H), 1.63 (s, 3H), 1.50 (s, 3H), 1.49 (s, 3H). ¹³C NMR (500 MHz, CDCl₃) δ 167.6, 134.7, 132.9, 131.9, 128.4, 85.9, 56.9, 45.7, 31.1, 25.9, 25.7, 14.8.

Analog 24:

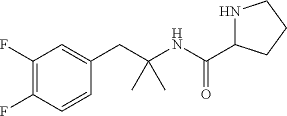 [SMILES: CC(C)(Cc1ccc(F)c(F)c1)NC(=O)C1CCCN1]

N-(1-(3,4-difluorophenyl)-2-methylpropan-2-yl)pyrrolidine-2-carboxamide: ¹H NMR (500 MHz, MeOD) δ 7.16 (m, 1H), 7.04 (m, 1H), 6.93 (m, 1H), 4.06 (m, 1H), 3.41 (m, 1H), 3.30 (m, 1H), 3.10 (d, 1H, J=13.4 Hz), 3.03 (d, 1H, J=13.4 Hz), 2.30 (m, 1H), 2.03 (m, 2H), 1.89 (m, 1H), 1.32 (s, 3H), 1.31 (s, 3H). ¹³C NMR (500 MHz, MeOD) δ 167.7, 150.3 (dd, J=71.7, 13.3 Hz), 148.3 (dd, J=71.3, 12.8 Hz), 135.4 (m), 126.0 (m), 118.6 (d, J=16.8 Hz), 116.3 (d, J=17.2 Hz), 60.0, 54.1, 45.9, 42.8, 29.9, 25.9, 25.8, 23.8.

### Example 4

### Evaluation of Leads in Permeability Assays for Oral Brain Uptake; In Vitro ADME/T Assays; Molecular Mechanism Studies

Validated ‘hits’ are evaluated in permeability assays using PAMPA and other in vitro assays that profile the properties of the molecule that modulate absorption distribution, metabolism & toxicity (ADME/T) assays. The analogs with the best profile can proceed to an in vivo oral brain uptake analysis.

### Preliminary Results

The initial screening on a small set of ˜1000 compounds demonstrates the feasibility of identifying ‘hits’ that are effective in the primary, secondary, and tertiary assays.

### Methods:

In vitro profiling assays that measure properties affecting absorption, distribution, metabolism, excretion and toxicity (ADME/T) are routinely employed to optimize the drug-like properties of analogues and to aid in the selection of compounds for further development. Selected compounds were thoroughly characterized in standard in vitro ADME/T assays to determine aqueous solubility, as well as chemical, plasma and metabolic stability, and membrane permeability. The compounds were then ranked according to the ideal profile. The goal was to identify compounds with the best in vitro properties and permeability, as well as brain uptake for further efficacy testing. The synthesized compounds were thoroughly tested in a panel of assays that we have implemented and use routinely, with each stage providing a go/no-go decision point based on stringent criteria encompassing activity/potency in a range of conditions as shown in Table 7.

TABLE 7
Cut-off values for in vitro ADM E/T assays and in vivo PK for
physiochemical and pharmacologic profiling of analogs.

| Property | Assays Available | Ideal matrics | Ref |
|---|---|---|---|
| Solubility | Chemiluminescence | >10 × EC₅₀ | Lipinski (2000) J. Pharmacol. |
| | | | Toxicol. Meth. 44(1): 235-249 |
| Protein | Rapid Equilibrium | <98.5% | Banker et at. (2003) J. Pharm. |
| Binding | Dialysis | Bound | Sci. 92(5): 967-974 |
| Metabolic | Liver Microsomes | T_{1/2} >0.5 hr | Gibson (1986) Biochem. |
| Stability | Or S9 Fraction | | Pharmacol. 35(24): 4431-4436; |
| | (various species) | | Guengerich (1989) J. Biol. |
| | | | Chem. 264(29): 17098-17205 |
| Permeability | CNS PAMPA | LogP (1.0 to | Kansy et at. (1998) J. Med. |
| | | 4.5); | Chem. 41(7): 1007-1010 |
| | | Kiam >0.65 | |
| Toxicity | MTT Cell-titer lo | ≧50 μM IC₅₀ | Mosmann (1983) J. Immunol. |
| | Multi-tox assay | | Meth. 65(1-2): 55-63 |
| In vivo | Comprehensive | B/P >0.25 | Korfmacher et at. (2001) Rapid |
| exposure | pharmokinetic (PK) | Cmax(brain) ≧ EC₅₀ | Commun. Mass. Spectrom. |
| | assessment @ 10 | | 15(5): 335-340; Mei et al. |
| | mpk | | (2006) AAPS J. 8(3): E493-500 |

Solubility, Protein Binding, Metabolic Stability & Cell Toxicity.

Solubility and protein binding are ascertained using chemiluminescence and filtration/dialysis assays routinely used in the lab (Banker et al. (2003) J. Pharm. Sci. 92(5): 967-974; Lipinski (2000) J. Pharmacol. Toxicol. Meth. 44(1): 235-249). For metabolic stability, the compound(s) is incubated with S9 fraction liver microsomes S9 fraction and stability measured by HPLC over a period of 1 h (Gibson (1986) Biochem. Pharmacol. 35(24): 4431-4436; Guengerich (1989) J. Biol. Chem. 264(29):17098-17205). For metabolic stability, the compound(s) is incubated with S9 fraction liver microsomes S9 fraction and stability measured by HPLC over a period of 1 h (Id.). Cell viability is assessed by ATP content using CellTiter Glo® (Promega Corp., Madison, Wis. (Kansy et al. (1998) J. Med. Chem. 41(7): 1007-1010).

In Vitro Permeability Testing (PAMPA).

Compounds were evaluated in the parallel artificial membrane permeability assay (PAMPA) using immobilized artificial membranes (IAM) and chromatography using, e.g., the IAM column from Regis technology (www.registech.com) using a Shimadzu HPLC system (Mosmann (1983) J. Immunol. Meth. 65(1-2): 55-63). Our data has shown that compounds with KI_{AMPerm}=K_{IAM}/(MW)>0.65 have increased brain permeability, and compounds with KI_{AMPerm}>1 have high brain/plasma ratios.

Brain Uptake Testing.

Pharmacokinetic (PK) analysis for CNS exposure studies can consist of a time course design to collect heparinized plasma and brains (Korfmacher et al. (2001) Rapid Commun. Mass. Spectrom. 15(5): 335-340; Mei et al. (2006) AAPS J. 8(3): E493-500; Spilman et al. (2014) Brain Res. 1551: 25-44). Five non-transgenic mice can be used for the PK studies, and 1, 2, 4, 6, and 8 hour time points taken. Following oral, and either sc or ip administration of the molecules at 10 mg/kg, plasma and brain levels of the compounds can be determined by quantitative LC/MS/MS methodology. Plasma samples can be precipitated with acetonitrile:methanol (1:1) cocktail containing an internal standard. The brain samples can be homogenized directly in ethylacetate or extracted from 5M guanidine homogenates with liquid-liquid method. The resulting supernatant can be evaporated to dryness and subjected to the LC/MS/MS analysis. For each compound 3 mice can be used for this analysis. The brain-to-plasma ratios and brain levels can then be calculated to identify the best candidate(s) for further efficacy testing.

Molecular Mechanism Studies.

The analogs that have good permeability can be evaluated for effects on SirT1, SirT2 and ADAM10 mRNA expression. As part of the mechanistic studies markers of mitochondrial respiration, glucogenesis and lipid anabolism that could be affected by SirT1 activation (Canto and Auwerx (2012) Pharmacol. Rev. 64(1):166-187) can be monitored. Quantitative proteomics experiments on candidate compounds treated in our cell models can be performed using Orbitrap XL configured for LC-MSMS.

### Example 5

### Efficacy Testing of Selected Leads in AD Models

Optimized compounds and analogs that have good brain plasma ratios (b/p>0.25) can be tested in the ApoE4-5XFAD mouse model. Acute and chronic efficacy testing of can be performed to determine optimum dose and pharmacodynamic correlations.

### In Vivo Testing.

Efficacy testing can be done to establish the compound(s) effects on behavioral and biochemical readouts and on disease progression in vivo in the mouse model. These studies can be performed as follows: ApoE4^{+/+}-5XAD^{+/-} (EFAD) mice can be used for compound testing and can be generated by cross-breeding 2 inbred mouse lines: the ApoE4 (Sullivan et al. (1997) J. Biol. Chem. 272(29): 17972-17980; Sullivan et al. (1980) J. Clin. Invest. 102(1): 130-135) line (APOE4-TR), where endogenous mouse ApoE is replaced by human ApoE4 under the control of the GFAP promoter allowing astrocytic expression; and the 5XFAD (Oakley et al. (2006) J. Neurosci. 26(40):10129-10140) Alzheimer's model line expressing human APP with three mutations (Swedish, K670N/M671L; Florida, I716V; and London, V717I) and presenilin 1 with two mutations (M146L and L286V) under control of the Thy-1 promoter and therefore allowing neuronal expression. A minimum of twelve 4-6 month-old EFAD male mice (Tai et al. (2013) J. Biol. Chem. 288(8): 5914-5926; Youmans et al. (2011) J. Neurosci. Meth. 196(1): 51-59; Youmans et al. (2012) J. Biol. Chem. 287(50): 41774-41786) per group—compound and vehicle control—can be used, as well as 12 NTg male vehicle-treated mice for a total of 36 mice per study. As proof-of-concept, candidate compounds can be tested at 10 mg/Kg/day or a dose that give brain levels adequate to elicit a pharmacodynamic response. If PK reveals brain levels are adequate with oral delivery, that method can be used; if not, sc or ip injection can be used.

Initial efficacy studies can be run for 28 days. If a compound shows subchronic efficacy, then longer studies—8 to 12 weeks—can be performed.

The compounds can be formulated similarly for injection or oral administration. Saline can be used when possible, or polyethylene glycol 400-ethanol-water (4:3:3, v/v) or hydroxyl-propyl-β-cyclodextrin mixtures.

Behavioral analysis can be performed pre-treatment and end-of-study and can include measurement of working object memory using the Novel Object Recognition testing paradigm, and spatial memory by the Novel Location Recognition paradigm. Morris Water Maze spatial memory assessment may be performed for mice in extended studies of compounds showing efficacy in initial studies.

As female mice show greater variability they need not be used in initial studies. However, as compound effects may differ according to gender, efficacy studies can be repeated using female mice if efficacy is seen in males. The readouts for biochemical efficacy can be performed using right brain and can include levels of the biomarkers SirT1 (AlphaLISA, Perkin-Elmer), sAPPα (AlphaLISA) sAPPβ (Life Technologies' ELISA), Aβ-40 and 1-42 (Life Technologies' ELISA) and p-tau (IP & AlphaLISA) and sAPPα/Aβ and sAPPα/p-Tau ratios. Immunoblotting can be used for determination of αCTF and βCTF.

Left hemi-brains can be used for immunohistochemical analysis of pathology after submersion fixation in 4% paraformaldehyde (PFA) and can include Aβplaque load (anti-Aβ antibodies), and astrocytic (anti-GFAP) and microglial (anti-Iba1 and other markers) inflammatory responses. Synaptic puncta can be labeled with antisynaptophysin to determine synaptic load.

Safety Panel Testing.

In vitro safety profiling services can be used to test for off-target interactions of advanced lead compounds. For example the SafetyScreen 44 offered by Eurofins Cerep (www.cerep.com) can be used for this testing. All the 44 selected targets, recommended by 4 major pharmaceutical companies1, are gathered in a cost-effective panel that associates robustness and strategy.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound according to the formula: 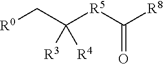
or a pharmaceutically acceptable salt thereof, wherein:
R⁸ is selected from the group consisting of 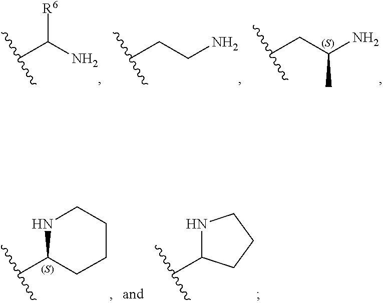
R⁰ is a substituted or unsubstituted cyclic or heterocycle selected from the group consisting of pyridine, pyrimidine, naphthalene, quinolone, isoquinoline, cinnoline, phenyl, substituted phenyl, oxazole, furan, isoxazole, thiazole, thiophene, pyrole, pyrazole, and imidazole;
R³ and R⁴ are independently selected from the group consisting of hydrogen, methyl, ethyl, propyl, and butyl, or R³ taken with R⁴ and the carbon joining R³ and R⁴ form cyclohexane or cyclobutane;
R⁵ is selected from the group consisting of O, NH, and NHR⁷, where R⁷ is a C1-C5 alkyl, or a cycloalkyl;
R⁶ is selected from the group consisting the R-group (side chain) of one of the 20 natural amino acids, phenylglycine, and norleucine; and
R⁶ is not CH₃, or R³ and R⁴ are not both CH₃, or when R⁶ is CH₃, said compound is not a compound selected from the group consisting of

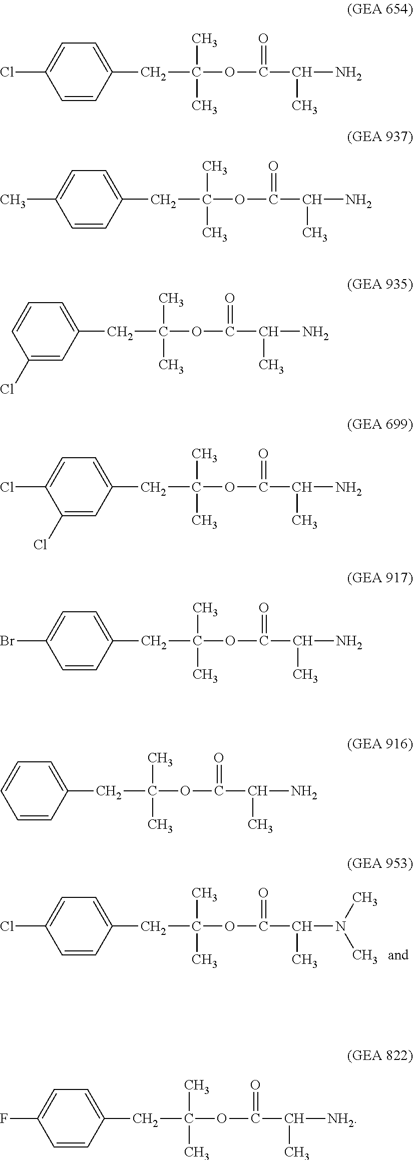

2. The compound of claim 1, wherein said compound is not any of compounds 1, 2, 4, 5, 6, 7, 8, 11, and 15 in Table 6.

3. The according to any one of claims 1-2, wherein said compound is a compound according to the formula 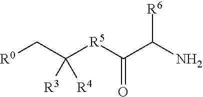
or a pharmaceutically acceptable salt thereof.

4. The compound according to any one of claim 1-3, wherein R³ and R⁴ are independently selected from the group consisting of hydrogen, methyl, ethyl, propyl, and butyl.

5. The according to any one of claims 1-4, wherein said compound has the formula 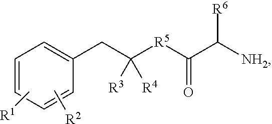
or a pharmaceutically acceptable salt thereof, wherein
R¹ and R² are independently selected from the group consisting of hydrogen, halogen, alkyl having 1, 2 or 3 carbon atoms, and alkoxy having 1, 2 or 3 carbon atoms.

6. The compound of claim 5, wherein said compound has the formula

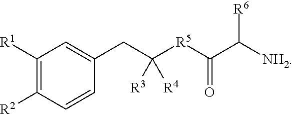

7. The compound according to any one of claims 1-6, wherein R³ is CH₃.

8. The compound of claim 7, wherein R⁴ is H.

9. The compound of claim 7, wherein R⁴ is CH₃.

10. The compound according to any one of claims 1-9, wherein R⁵ is O.

11. The compound according to any one of claims 1-9, wherein R⁵ is NHR⁷.

12. The compound according to any one of claims 1-9, wherein R⁵ is NH.

13. The compound according to any one of claims 1-12, wherein R¹ and R² are independently selected from the group consisting of hydrogen, halogen, and CH₃.

14. The compound of claim 13, wherein R¹ and R² are independently selected from the group consisting of H, Cl, and F.

15. The compound of claim 13, wherein R¹ and R² are both Cl.

16. The compound of claim 13, wherein R¹ and R² are both F.

17. The compound of claim 13, wherein R¹ is Cl and R² is F, or R¹ is F and R² is Cl.

18. The compound of claim 13, wherein R¹ is H and R² is F.

19. The compound of claim 13, wherein R¹ is H and R² is Cl.

20. The compound of claim 13, wherein R¹ is H and R² is CH₃.

21. The compound according to any one of claims 1-20, wherein R⁶ is selected from the group consisting of H, CH₃, —CH(CH₃)₂, —CH₂—CH(CH₃)₂, —CH₂-phenyl, CH2-substituted phenyl, —CH(CH₃)—CH₂CH₃, -phenyl, substituted phenyl, and —CH₂—CH₂—CH₂—CH₃.

22. The compound according to any one of claims 1-20, wherein R⁶ is H.

23. The compound according to any one of claims 1-20, wherein R⁶ is CH₃.

24. The compound according to any one of claims 1-20, wherein R⁶ is—CH(CH₃)₂.

25. The compound according to any one of claims 1-20, wherein R⁶ is —CH₂—CH(CH₃)₂.

26. The compound according to any one of claims 1-20, wherein R⁶ is —CH₂-phenyl.

27. The compound according to any one of claims 1-20, wherein R⁶ is —CH(CH₃)—CH₂CH₃.

28. The compound according to any one of claims 1-20, wherein R⁶ is -phenyl.

29. The compound according to any one of claims 1-20, wherein R⁶ is —CH₂—CH₂—CH₂—CH₃.

30. The compound of claim 1, wherein said compound is a compound is any one of compounds 1 through 24 in Table 6, or any one of compounds 3, 9, 10, 12, 13, 14, 16, 17, 18, 19, 20, 21, 22, 23, or 24 in Table 6, or a pharmaceutically acceptable salt thereof.

31. The compound according to any one of claims 1-30, wherein said compound is a substantially pure S enantiomer or a substantially pure R enantiomer.

32. A pharmaceutical formulation comprising one or more compounds according to any one of claims 1-31 and a pharmaceutically acceptable diluent or excipient.

33. The pharmaceutical formulation of claim 32, wherein said formulation is a unit dosage formulation.

34. The formulation according to any one of claims 32-33, wherein said formulation is compounded for administration via a route selected from the group consisting of oral delivery, isophoretic delivery, transdermal delivery, parenteral delivery, aerosol administration, administration via inhalation, intravenous administration, and rectal administration.

35. A method of increasing the expression of SirT1, and/or increasing the expression of ADAM10, and/or increasing sAPPα, and/or decreasing p-tau in a mammal; and/or
normalizing ApoE4 mediated effects on SirT1, normalizing SirT1/SirT2 ratios, and/or normalizing APP processing in a mammal; and/or
promoting the processing of amyloid precursor protein (APP) by the non-amyloidogenic pathway in a mammal; and/or
preventing or delaying the onset of a pre-Alzheimer's condition and/or cognitive dysfunction, and/or ameliorating one or more symptoms of a pre-Alzheimer's condition and/or cognitive dysfunction, or preventing or delaying the progression of a pre-Alzheimer's condition or cognitive dysfunction to Alzheimer's disease in a mammal; and/or
ameliorating one or more symptoms of Alzheimer's disease, and/or reversing Alzheimer's disease, and/or reducing the rate of progression of Alzheimer's disease in a mammal; and/or
treating diabetes and/or metabolic syndrome in a mammal; and/or
increasing the lifespan and/or healthspan of a mammal, said method comprising:
administering to said mammal an effective amount of one or more compounds according to any one of claims 1-31 and/or a compound selected from the group consisting of alaproclate keto analogues (e.g., 2-amino-6-(4-chlorophenyl)-5,5-dimethyl-3-hexanone and 5-amino-1-(4-chlorophenyl)-2,2-dimethyl-3-hexanone), isopropyl alaproclate analogues (e.g., 2-(4-clorophenyl)-1,1-dimethyl 2-amino-3-methylbutanoate, 2-diethylaminoethyl 2,2-diphenylpentanoate (proadifen), 2-(4-chlorophenyl)-1,1-dimethylethyl 2-amino-3-methylbutanoate (GEA 857), 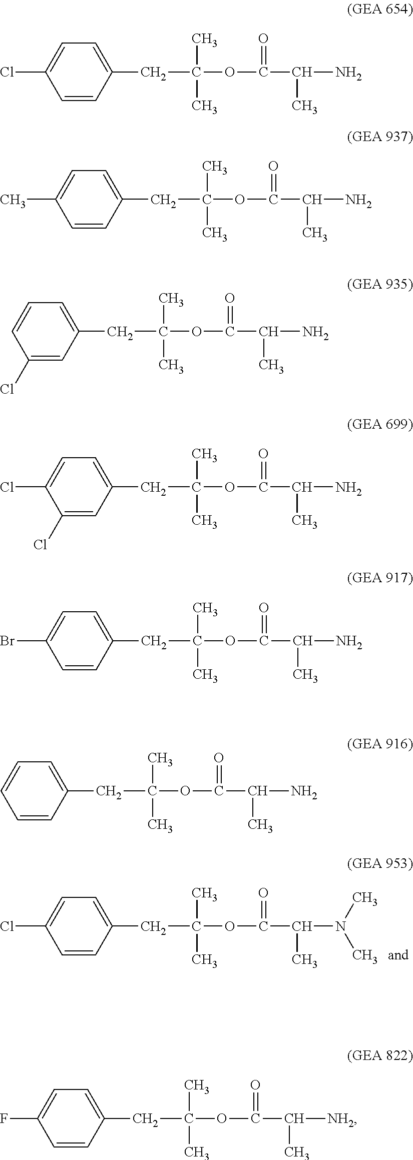
and/or a formulation according to any one of claims 32-34.

36. The method of claim 35, wherein said compound comprises alaproclate.

37. The method of claim 35, wherein said compound comprises any one of compounds 1-24 in Table 6.

38. The method according to any one of claims 35-37, wherein said method increases the expression of SirT1 in said mammal.

39. The method according to any one of claims 35-38, wherein said method increases the expression of ADAM10 in said mammal.

40. The method according to any one of claims 35-39, wherein said method increases sAPPα in said mammal.

41. The method according to any one of claims 35-40, wherein said method decreases p-tau in said mammal.

42. The method according to any one of claims 35-41, wherein said mammal is a human.

43. The method according to any one of claims 35-42, wherein, wherein said method is a method of preventing or delaying the transition from a cognitively asymptomatic pre-Alzheimer's condition to a pre-Alzheimer's cognitive dysfunction.

44. The method according to any one of claims 35-42, wherein said method is a method of preventing or delaying the onset of a pre-Alzheimer's cognitive dysfunction.

45. The method according to any one of claims 35-42, wherein said method comprises ameliorating one or more symptoms of a pre-Alzheimer's cognitive dysfunction.

46. The method according to any one of claims 35-42, wherein said method comprises preventing or delaying the progression of a pre-Alzheimer's cognitive dysfunction to Alzheimer's disease.

47. The method of claim 46, wherein said method delays or prevents the progression of MCI to Alzheimer's disease.

48. The method according to any one of claims of claim 35-42, wherein said method is a method of ameliorating one or more symptoms of Alzheimer's disease, and/or reversing Alzheimer's disease, and/or reducing the rate of progression of Alzheimer's disease in a mammal.

49. The method according to any one of claims 35-48, wherein:
the mammal has a familial risk for having Alzheimer's disease; and/or
the mammal has a familial Alzheimer's disease (FAD) mutation; and/or
the mammal has one copy of the ApoE4 allele; and/or
the mammal has two copies of the ApoE4 allele; and/or
the mammal exhibits biomarker positivity of Aβ in a clinically normal human mammal age 50 or older; and/or
the mammal exhibits asymptomatic cerebral amyloidosis; and/or
the mammal exhibits cerebral amyloidosis in combination with downstream neurodegeneration; and/or
the mammal is cognitively asymptomatic; and/or
the mammal exhibits cerebral amyloidosis in combination with downstream neurodegeneration and subtle cognitive/behavioral decline; and/or
the mammal is a mammal diagnosed with mild cognitive impairment; and/or
the mammal shows a clinical dementia rating above zero and below about 1.5; and/or
the mammal is not diagnosed as at risk for a neurological disease or disorder other than Alzheimer's disease.

50. The method according to any one of claims 35-49, wherein said administration:
produces a reduction in the CSF of levels of one or more components selected from the group consisting of total-Tau (tTau), phospho-Tau (pTau), APPneo, soluble Aβ40, pTau/Aβ42 ratio and tTau/Aβ42 ratio, and/or an increase in the CSF of levels of one or more components selected from the group consisting of Aβ42/Aβ40 ratio, Aβ42/Aβ38 ratio, sAPPα, sAPPα/sAPPβ ratio, sAPPα/Aβ40 ratio, and sAPPα/Aβ42 ratio; and/or
produces an increase in plasma levels of SirT1 or normalizes the SirT1/SirT2 ratios; and/or
produces a reduction of the plaque load in the brain of the mammal; and/or
produces a reduction in the rate of plaque formation in the brain of the mammal; and/or
produces an improvement in the cognitive abilities of the mammal; and/or
produces an improvement in, a stabilization of, or a reduction in the rate of decline of the clinical dementia rating (CDR) of the mammal; and/or
where the mammal is a human and said administration produces a perceived improvement in quality of life by the human.

51. The method according to any one of claims 35-50, wherein the compound(s) are administered via a route selected from the group consisting of oral delivery, isophoretic delivery, transdermal delivery, parenteral delivery, aerosol administration, administration via inhalation, intravenous administration, and rectal administration.

52. The method according to any one of claims 35-50, wherein the compound is administered orally.

53. The method according to any one of claims 35-52, wherein the administering is over a period of at least three weeks, over a period of at least 6 months.

54. The method according to any one of claims 35-53, wherein the compound(s) are formulated for administration via a route selected from the group consisting of isophoretic delivery, transdermal delivery, aerosol administration, administration via inhalation, oral administration, intravenous administration, and rectal administration.
